SEQUENCE LISTING (937 sequences)

SEQ ID NO: 1 - DNA, 20 bp, synthetic construct
  misc_feature 1..20: note = Synthetic Polynucleotide
  source 1..20: mol_type = other DNA
taatacgactcactataggg

SEQ ID NO: 2 - DNA, 20 bp, synthetic construct
  misc_feature 1..20: note = Synthetic Polynucleotide
  source 1..20: mol_type = other DNA
caggcatccgagaggtctgg

SEQ ID NO: 3 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagcagctctacaagtgcggccatttggttcattgcgcttctccggaaacattagc

SEQ ID NO: 4 - DNA, 20 bp, synthetic construct
  misc_feature 1..20: note = Synthetic Polynucleotide
  source 1..20: mol_type = other DNA
cggtaggaccctacacatcc

SEQ ID NO: 5 - DNA, 20 bp, synthetic construct
  misc_feature 1..20: note = Synthetic Polynucleotide
  source 1..20: mol_type = other DNA
tgtggcgcctaaccatacac

SEQ ID NO: 6 - DNA, 20 bp, synthetic construct
  misc_feature 1..20: note = Synthetic Polynucleotide
  source 1..20: mol_type = other DNA
gacaccgagctagaattcgg

SEQ ID NO: 7 - DNA, 20 bp, synthetic construct
  misc_feature 1..20: note = Synthetic Polynucleotide
  source 1..20: mol_type = other DNA
tgtcttgggtcctcttacgg

SEQ ID NO: 8 - DNA, 20 bp, synthetic construct
  misc_feature 1..20: note = Synthetic Polynucleotide
  source 1..20: mol_type = other DNA
tatcacgtgaggctccgtgt

SEQ ID NO: 9 - DNA, 20 bp, synthetic construct
  misc_feature 1..20: note = Synthetic Polynucleotide
  source 1..20: mol_type = other DNA
gtctacggatccttgcgtgt

SEQ ID NO: 10 - DNA, 20 bp, synthetic construct
  misc_feature 1..20: note = Synthetic Polynucleotide
  source 1..20: mol_type = other DNA
acgggtacattggatccact

SEQ ID NO: 11 - DNA, 20 bp, synthetic construct
  misc_feature 1..20: note = Synthetic Polynucleotide
  source 1..20: mol_type = other DNA
aagacgtggtccaccctgat

SEQ ID NO: 12 - DNA, 20 bp, synthetic construct
  misc_feature 1..20: note = Synthetic Polynucleotide
  source 1..20: mol_type = other DNA
tgccaggtccactacttggg

SEQ ID NO: 13 - DNA, 20 bp, synthetic construct
  misc_feature 1..20: note = Synthetic Polynucleotide
  source 1..20: mol_type = other DNA
gacccgaaggagaatgcaaa

SEQ ID NO: 14 - DNA, 20 bp, synthetic construct
  misc_feature 1..20: note = Synthetic Polynucleotide
  source 1..20: mol_type = other DNA
atgtcgtctctcagggcttt

SEQ ID NO: 15 - DNA, 20 bp, synthetic construct
  misc_feature 1..20: note = Synthetic Polynucleotide
  source 1..20: mol_type = other DNA
aagggcggaagcactctctt

SEQ ID NO: 16 - DNA, 20 bp, synthetic construct
  misc_feature 1..20: note = Synthetic Polynucleotide
  source 1..20: mol_type = other DNA
tggtcccgcatttgtacctt

SEQ ID NO: 17 - DNA, 20 bp, synthetic construct
  misc_feature 1..20: note = Synthetic Polynucleotide
  source 1..20: mol_type = other DNA
aagtccgtctgctaagccat

SEQ ID NO: 18 - DNA, 20 bp, synthetic construct
  misc_feature 1..20: note = Synthetic Polynucleotide
  source 1..20: mol_type = other DNA
cttccagagtcctggcatga

SEQ ID NO: 19 - DNA, 20 bp, synthetic construct
  misc_feature 1..20: note = Synthetic Polynucleotide
  source 1..20: mol_type = other DNA
tctaacgagctccgtggttg

SEQ ID NO: 20 - DNA, 20 bp, synthetic construct
  misc_feature 1..20: note = Synthetic Polynucleotide
  source 1..20: mol_type = other DNA
cctgggcaggaaacaacact

SEQ ID NO: 21 - DNA, 20 bp, synthetic construct
  misc_feature 1..20: note = Synthetic Polynucleotide
  source 1..20: mol_type = other DNA
gcgcacaccttagtcatagc

SEQ ID NO: 22 - DNA, 20 bp, synthetic construct
  misc_feature 1..20: note = Synthetic Polynucleotide
  source 1..20: mol_type = other DNA
gtgcgattgtcacatgtgct

SEQ ID NO: 23 - DNA, 20 bp, synthetic construct
  misc_feature 1..20: note = Synthetic Polynucleotide
  source 1..20: mol_type = other DNA
ggcttgtcctcaattatccg

SEQ ID NO: 24 - DNA, 20 bp, synthetic construct
  misc_feature 1..20: note = Synthetic Polynucleotide
  source 1..20: mol_type = other DNA
cccacgtgacttgacttctc

SEQ ID NO: 25 - DNA, 20 bp, synthetic construct
  misc_feature 1..20: note = Synthetic Polynucleotide
  source 1..20: mol_type = other DNA
cctctacatgctccggatcc

SEQ ID NO: 26 - DNA, 20 bp, synthetic construct
  misc_feature 1..20: note = Synthetic Polynucleotide
  source 1..20: mol_type = other DNA
gaatagtaacccggtcgcaa

SEQ ID NO: 27 - DNA, 20 bp, synthetic construct
  misc_feature 1..20: note = Synthetic Polynucleotide
  source 1..20: mol_type = other DNA
tgggccactagtcttcgcta

SEQ ID NO: 28 - DNA, 20 bp, synthetic construct
  misc_feature 1..20: note = Synthetic Polynucleotide
  source 1..20: mol_type = other DNA
cggcacgtaccctttacagg

SEQ ID NO: 29 - DNA, 20 bp, synthetic construct
  misc_feature 1..20: note = Synthetic Polynucleotide
  source 1..20: mol_type = other DNA
gatcagtgagcctcaccaag

SEQ ID NO: 30 - DNA, 20 bp, synthetic construct
  misc_feature 1..20: note = Synthetic Polynucleotide
  source 1..20: mol_type = other DNA
tggtatgtgagctctttggg

SEQ ID NO: 31 - DNA, 20 bp, synthetic construct
  misc_feature 1..20: note = Synthetic Polynucleotide
  source 1..20: mol_type = other DNA
agagtcacgcatacttggga

SEQ ID NO: 32 - DNA, 20 bp, synthetic construct
  misc_feature 1..20: note = Synthetic Polynucleotide
  source 1..20: mol_type = other DNA
gggtcaggcgtcatagattg

SEQ ID NO: 33 - DNA, 20 bp, synthetic construct
  misc_feature 1..20: note = Synthetic Polynucleotide
  source 1..20: mol_type = other DNA
agaatgtggaggacatgtgg

SEQ ID NO: 34 - DNA, 20 bp, synthetic construct
  misc_feature 1..20: note = Synthetic Polynucleotide
  source 1..20: mol_type = other DNA
tcggactgtgagacatttcc

SEQ ID NO: 35 - DNA, 20 bp, synthetic construct
  misc_feature 1..20: note = Synthetic Polynucleotide
  source 1..20: mol_type = other DNA
atcaaatgcctcggtcgttg

SEQ ID NO: 36 - DNA, 20 bp, synthetic construct
  misc_feature 1..20: note = Synthetic Polynucleotide
  source 1..20: mol_type = other DNA
gcaatgctatgctgggacaa

SEQ ID NO: 37 - DNA, 20 bp, synthetic construct
  misc_feature 1..20: note = Synthetic Polynucleotide
  source 1..20: mol_type = other DNA
atggaggccgttctctgaca

SEQ ID NO: 38 - DNA, 20 bp, synthetic construct
  misc_feature 1..20: note = Synthetic Polynucleotide
  source 1..20: mol_type = other DNA
gcagatgggtgcattcattc

SEQ ID NO: 39 - DNA, 20 bp, synthetic construct
  misc_feature 1..20: note = Synthetic Polynucleotide
  source 1..20: mol_type = other DNA
tatgcactctgcggatgacc

SEQ ID NO: 40 - DNA, 20 bp, synthetic construct
  misc_feature 1..20: note = Synthetic Polynucleotide
  source 1..20: mol_type = other DNA
gacccgggagttatgatacg

SEQ ID NO: 41 - DNA, 20 bp, synthetic construct
  misc_feature 1..20: note = Synthetic Polynucleotide
  source 1..20: mol_type = other DNA
ctcgggtaggctatctccaa

SEQ ID NO: 42 - DNA, 20 bp, synthetic construct
  misc_feature 1..20: note = Synthetic Polynucleotide
  source 1..20: mol_type = other DNA
ctgcaggtgctctagatgga

SEQ ID NO: 43 - DNA, 20 bp, synthetic construct
  misc_feature 1..20: note = Synthetic Polynucleotide
  source 1..20: mol_type = other DNA
gtccaggctcgtcttctcga

SEQ ID NO: 44 - DNA, 20 bp, synthetic construct
  misc_feature 1..20: note = Synthetic Polynucleotide
  source 1..20: mol_type = other DNA
cttacctgagcgcagttcga

SEQ ID NO: 45 - DNA, 20 bp, synthetic construct
  misc_feature 1..20: note = Synthetic Polynucleotide
  source 1..20: mol_type = other DNA
gttgttcgatccctccacca

SEQ ID NO: 46 - DNA, 20 bp, synthetic construct
  misc_feature 1..20: note = Synthetic Polynucleotide
  source 1..20: mol_type = other DNA
cggcagaggataatcctagc

SEQ ID NO: 47 - DNA, 20 bp, synthetic construct
  misc_feature 1..20: note = Synthetic Polynucleotide
  source 1..20: mol_type = other DNA
gggagtccggatgttagtgc

SEQ ID NO: 48 - DNA, 20 bp, synthetic construct
  misc_feature 1..20: note = Synthetic Polynucleotide
  source 1..20: mol_type = other DNA
cgcatctatgctccgcttac

SEQ ID NO: 49 - DNA, 20 bp, synthetic construct
  misc_feature 1..20: note = Synthetic Polynucleotide
  source 1..20: mol_type = other DNA
aacgcgctacccaattctag

SEQ ID NO: 50 - DNA, 20 bp, synthetic construct
  misc_feature 1..20: note = Synthetic Polynucleotide
  source 1..20: mol_type = other DNA
tactggacgattcccgactg

SEQ ID NO: 51 - DNA, 20 bp, synthetic construct
  misc_feature 1..20: note = Synthetic Polynucleotide
  source 1..20: mol_type = other DNA
agcgtcgtacccagttaagc

SEQ ID NO: 52 - DNA, 20 bp, synthetic construct
  misc_feature 1..20: note = Synthetic Polynucleotide
  source 1..20: mol_type = other DNA
gagattcacgccctcatgag

SEQ ID NO: 53 - DNA, 20 bp, synthetic construct
  misc_feature 1..20: note = Synthetic Polynucleotide
  source 1..20: mol_type = other DNA
ccgctagcgttacctctacc

SEQ ID NO: 54 - DNA, 20 bp, synthetic construct
  misc_feature 1..20: note = Synthetic Polynucleotide
  source 1..20: mol_type = other DNA
ccacaccgtatgcatttctg

SEQ ID NO: 55 - DNA, 20 bp, synthetic construct
  misc_feature 1..20: note = Synthetic Polynucleotide
  source 1..20: mol_type = other DNA
ccttagcgcacaaagagacg

SEQ ID NO: 56 - DNA, 20 bp, synthetic construct
  misc_feature 1..20: note = Synthetic Polynucleotide
  source 1..20: mol_type = other DNA
gtcctgatggctttctcacg

SEQ ID NO: 57 - DNA, 20 bp, synthetic construct
  misc_feature 1..20: note = Synthetic Polynucleotide
  source 1..20: mol_type = other DNA
ggtcgtcggtagatcattgc

SEQ ID NO: 58 - DNA, 20 bp, synthetic construct
  misc_feature 1..20: note = Synthetic Polynucleotide
  source 1..20: mol_type = other DNA
agagccggtatgatccatcg

SEQ ID NO: 59 - DNA, 20 bp, synthetic construct
  misc_feature 1..20: note = Synthetic Polynucleotide
  source 1..20: mol_type = other DNA
ccggacgaaggttgatatcc

SEQ ID NO: 60 - DNA, 20 bp, synthetic construct
  misc_feature 1..20: note = Synthetic Polynucleotide
  source 1..20: mol_type = other DNA
acccggtacctgttatcacg

SEQ ID NO: 61 - DNA, 20 bp, synthetic construct
  misc_feature 1..20: note = Synthetic Polynucleotide
  source 1..20: mol_type = other DNA
atcccgtctcgttctatggg

SEQ ID NO: 62 - DNA, 20 bp, synthetic construct
  misc_feature 1..20: note = Synthetic Polynucleotide
  source 1..20: mol_type = other DNA
caggtgagggtctctccttc

SEQ ID NO: 63 - DNA, 20 bp, synthetic construct
  misc_feature 1..20: note = Synthetic Polynucleotide
  source 1..20: mol_type = other DNA
ccttatggaaacgtgatgcc

SEQ ID NO: 64 - DNA, 20 bp, synthetic construct
  misc_feature 1..20: note = Synthetic Polynucleotide
  source 1..20: mol_type = other DNA
ggctgggcatacatagacct

SEQ ID NO: 65 - DNA, 20 bp, synthetic construct
  misc_feature 1..20: note = Synthetic Polynucleotide
  source 1..20: mol_type = other DNA
agtggcggctattaccggat

SEQ ID NO: 66 - DNA, 20 bp, synthetic construct
  misc_feature 1..20: note = Synthetic Polynucleotide
  source 1..20: mol_type = other DNA
tgataacgtccgctcgttga

SEQ ID NO: 67 - DNA, 20 bp, synthetic construct
  misc_feature 1..20: note = Synthetic Polynucleotide
  source 1..20: mol_type = other DNA
tacatcgttgaggcccgttt

SEQ ID NO: 68 - DNA, 20 bp, synthetic construct
  misc_feature 1..20: note = Synthetic Polynucleotide
  source 1..20: mol_type = other DNA
cgttgtggcatcagctagag

SEQ ID NO: 69 - DNA, 20 bp, synthetic construct
  misc_feature 1..20: note = Synthetic Polynucleotide
  source 1..20: mol_type = other DNA
actggcccacacacttacct

SEQ ID NO: 70 - DNA, 20 bp, synthetic construct
  misc_feature 1..20: note = Synthetic Polynucleotide
  source 1..20: mol_type = other DNA
atcgggtcacggaatatgac

SEQ ID NO: 71 - DNA, 20 bp, synthetic construct
  misc_feature 1..20: note = Synthetic Polynucleotide
  source 1..20: mol_type = other DNA
cgtaggttcacagatttgcg

SEQ ID NO: 72 - DNA, 20 bp, synthetic construct
  misc_feature 1..20: note = Synthetic Polynucleotide
  source 1..20: mol_type = other DNA
tagggcacgagaaggtatcc

SEQ ID NO: 73 - DNA, 20 bp, synthetic construct
  misc_feature 1..20: note = Synthetic Polynucleotide
  source 1..20: mol_type = other DNA
actagcttgtatcgccggat

SEQ ID NO: 74 - DNA, 20 bp, synthetic construct
  misc_feature 1..20: note = Synthetic Polynucleotide
  source 1..20: mol_type = other DNA
cggcggtttgatattcgaag

SEQ ID NO: 75 - DNA, 20 bp, synthetic construct
  misc_feature 1..20: note = Synthetic Polynucleotide
  source 1..20: mol_type = other DNA
gccgagtgtttatgagcaag

SEQ ID NO: 76 - DNA, 20 bp, synthetic construct
  misc_feature 1..20: note = Synthetic Polynucleotide
  source 1..20: mol_type = other DNA
tctccgaagatccgatatcg

SEQ ID NO: 77 - DNA, 20 bp, synthetic construct
  misc_feature 1..20: note = Synthetic Polynucleotide
  source 1..20: mol_type = other DNA
gatgcagggcctaattaacg

SEQ ID NO: 78 - DNA, 20 bp, synthetic construct
  misc_feature 1..20: note = Synthetic Polynucleotide
  source 1..20: mol_type = other DNA
tgacgcggctaaatactgac

SEQ ID NO: 79 - DNA, 20 bp, synthetic construct
  misc_feature 1..20: note = Synthetic Polynucleotide
  source 1..20: mol_type = other DNA
gcaatgcacgctctcctagt

SEQ ID NO: 80 - DNA, 20 bp, synthetic construct
  misc_feature 1..20: note = Synthetic Polynucleotide
  source 1..20: mol_type = other DNA
tggcgtcagatggattagga

SEQ ID NO: 81 - DNA, 20 bp, synthetic construct
  misc_feature 1..20: note = Synthetic Polynucleotide
  source 1..20: mol_type = other DNA
gttggcgggagactaagaag

SEQ ID NO: 82 - DNA, 20 bp, synthetic construct
  misc_feature 1..20: note = Synthetic Polynucleotide
  source 1..20: mol_type = other DNA
tcgcgtctcgtccttctacc

SEQ ID NO: 83 - DNA, 20 bp, synthetic construct
  misc_feature 1..20: note = Synthetic Polynucleotide
  source 1..20: mol_type = other DNA
cggtcacgtcggaaataacc

SEQ ID NO: 84 - DNA, 20 bp, synthetic construct
  misc_feature 1..20: note = Synthetic Polynucleotide
  source 1..20: mol_type = other DNA
attaacgggccaggttactg

SEQ ID NO: 85 - DNA, 20 bp, synthetic construct
  misc_feature 1..20: note = Synthetic Polynucleotide
  source 1..20: mol_type = other DNA
attgtatggaggcgccctat

SEQ ID NO: 86 - DNA, 20 bp, synthetic construct
  misc_feature 1..20: note = Synthetic Polynucleotide
  source 1..20: mol_type = other DNA
gccagctttccaagattcag

SEQ ID NO: 87 - DNA, 20 bp, synthetic construct
  misc_feature 1..20: note = Synthetic Polynucleotide
  source 1..20: mol_type = other DNA
acaaccgcgtgttacaaggc

SEQ ID NO: 88 - DNA, 20 bp, synthetic construct
  misc_feature 1..20: note = Synthetic Polynucleotide
  source 1..20: mol_type = other DNA
gctaatgtttccggagaagc

SEQ ID NO: 89 - DNA, 20 bp, synthetic construct
  misc_feature 1..20: note = Synthetic Polynucleotide
  source 1..20: mol_type = other DNA
gaagtatccggcatcacagc

SEQ ID NO: 90 - DNA, 20 bp, synthetic construct
  misc_feature 1..20: note = Synthetic Polynucleotide
  source 1..20: mol_type = other DNA
tcgttacggactttcacgac

SEQ ID NO: 91 - DNA, 20 bp, synthetic construct
  misc_feature 1..20: note = Synthetic Polynucleotide
  source 1..20: mol_type = other DNA
gccattctgacatacccaga

SEQ ID NO: 92 - DNA, 20 bp, synthetic construct
  misc_feature 1..20: note = Synthetic Polynucleotide
  source 1..20: mol_type = other DNA
gtatttgaaccggccagctg

SEQ ID NO: 93 - DNA, 20 bp, synthetic construct
  misc_feature 1..20: note = Synthetic Polynucleotide
  source 1..20: mol_type = other DNA
ggttgtgggccatatccaat

SEQ ID NO: 94 - DNA, 20 bp, synthetic construct
  misc_feature 1..20: note = Synthetic Polynucleotide
  source 1..20: mol_type = other DNA
ggcgttccatcgaaactcta

SEQ ID NO: 95 - DNA, 20 bp, synthetic construct
  misc_feature 1..20: note = Synthetic Polynucleotide
  source 1..20: mol_type = other DNA
ggccatcgacttagattcca

SEQ ID NO: 96 - DNA, 20 bp, synthetic construct
  misc_feature 1..20: note = Synthetic Polynucleotide
  source 1..20: mol_type = other DNA
ggtgctcctgccattatagg

SEQ ID NO: 97 - DNA, 20 bp, synthetic construct
  misc_feature 1..20: note = Synthetic Polynucleotide
  source 1..20: mol_type = other DNA
tgtcgacgtgcggaaagtag

SEQ ID NO: 98 - DNA, 20 bp, synthetic construct
  misc_feature 1..20: note = Synthetic Polynucleotide
  source 1..20: mol_type = other DNA
gccgcacgagtatgctactg

SEQ ID NO: 99 - DNA, 20 bp, synthetic construct
  misc_feature 1..20: note = Synthetic Polynucleotide
  source 1..20: mol_type = other DNA
tgctgccgcttactactgct

SEQ ID NO: 100 - DNA, 20 bp, synthetic construct
  misc_feature 1..20: note = Synthetic Polynucleotide
  source 1..20: mol_type = other DNA
tagcactcttctggccatcg

SEQ ID NO: 101 - DNA, 20 bp, synthetic construct
  misc_feature 1..20: note = Synthetic Polynucleotide
  source 1..20: mol_type = other DNA
cggtaatcggttcaccagtg

SEQ ID NO: 102 - DNA, 20 bp, synthetic construct
  misc_feature 1..20: note = Synthetic Polynucleotide
  source 1..20: mol_type = other DNA
tttagtgcctggctccgttg

SEQ ID NO: 103 - DNA, 20 bp, synthetic construct
  misc_feature 1..20: note = Synthetic Polynucleotide
  source 1..20: mol_type = other DNA
ggagttacagccactttcgg

SEQ ID NO: 104 - DNA, 20 bp, synthetic construct
  misc_feature 1..20: note = Synthetic Polynucleotide
  source 1..20: mol_type = other DNA
gtgtggacggaacctgacaa

SEQ ID NO: 105 - DNA, 20 bp, synthetic construct
  misc_feature 1..20: note = Synthetic Polynucleotide
  source 1..20: mol_type = other DNA
cacacgtggaattgttctgc

SEQ ID NO: 106 - DNA, 20 bp, synthetic construct
  misc_feature 1..20: note = Synthetic Polynucleotide
  source 1..20: mol_type = other DNA
cttgtacatggagggcgact

SEQ ID NO: 107 - DNA, 20 bp, synthetic construct
  misc_feature 1..20: note = Synthetic Polynucleotide
  source 1..20: mol_type = other DNA
aatccggtgtacaggttccc

SEQ ID NO: 108 - DNA, 20 bp, synthetic construct
  misc_feature 1..20: note = Synthetic Polynucleotide
  source 1..20: mol_type = other DNA
aggtgcgacgatatacacca

SEQ ID NO: 109 - DNA, 20 bp, synthetic construct
  misc_feature 1..20: note = Synthetic Polynucleotide
  source 1..20: mol_type = other DNA
actgcatatgaccgctgcaa

SEQ ID NO: 110 - DNA, 20 bp, synthetic construct
  misc_feature 1..20: note = Synthetic Polynucleotide
  source 1..20: mol_type = other DNA
ccttccattagccgagatca

SEQ ID NO: 111 - DNA, 20 bp, synthetic construct
  misc_feature 1..20: note = Synthetic Polynucleotide
  source 1..20: mol_type = other DNA
gacgcgaggagtgatcgact

SEQ ID NO: 112 - DNA, 20 bp, synthetic construct
  misc_feature 1..20: note = Synthetic Polynucleotide
  source 1..20: mol_type = other DNA
cgaccctcacacatttgggt

SEQ ID NO: 113 - DNA, 20 bp, synthetic construct
  misc_feature 1..20: note = Synthetic Polynucleotide
  source 1..20: mol_type = other DNA
cgctcccgtattcttctgtg

SEQ ID NO: 114 - DNA, 20 bp, synthetic construct
  misc_feature 1..20: note = Synthetic Polynucleotide
  source 1..20: mol_type = other DNA
cagggatagtcaggccagtg

SEQ ID NO: 115 - DNA, 20 bp, synthetic construct
  misc_feature 1..20: note = Synthetic Polynucleotide
  source 1..20: mol_type = other DNA
cgtaatgagtgcttcgccat

SEQ ID NO: 116 - DNA, 20 bp, synthetic construct
  misc_feature 1..20: note = Synthetic Polynucleotide
  source 1..20: mol_type = other DNA
ctcgtctcgtggaacacatg

SEQ ID NO: 117 - DNA, 20 bp, synthetic construct
  misc_feature 1..20: note = Synthetic Polynucleotide
  source 1..20: mol_type = other DNA
tatgctgggcagtaatcagg

SEQ ID NO: 118 - DNA, 20 bp, synthetic construct
  misc_feature 1..20: note = Synthetic Polynucleotide
  source 1..20: mol_type = other DNA
gcatctcgtagcatcctgct

SEQ ID NO: 119 - DNA, 20 bp, synthetic construct
  misc_feature 1..20: note = Synthetic Polynucleotide
  source 1..20: mol_type = other DNA
agtatggtaccgggaacagc

SEQ ID NO: 120 - DNA, 20 bp, synthetic construct
  misc_feature 1..20: note = Synthetic Polynucleotide
  source 1..20: mol_type = other DNA
accattctcgcaactcgcta

SEQ ID NO: 121 - DNA, 20 bp, synthetic construct
  misc_feature 1..20: note = Synthetic Polynucleotide
  source 1..20: mol_type = other DNA
cgtgggcgaagtacttggtc

SEQ ID NO: 122 - DNA, 20 bp, synthetic construct
  misc_feature 1..20: note = Synthetic Polynucleotide
  source 1..20: mol_type = other DNA
ccggacgtcttcgataatgc

SEQ ID NO: 123 - DNA, 20 bp, synthetic construct
  misc_feature 1..20: note = Synthetic Polynucleotide
  source 1..20: mol_type = other DNA
tgatgctctttgcagttcgg

SEQ ID NO: 124 - DNA, 20 bp, synthetic construct
  misc_feature 1..20: note = Synthetic Polynucleotide
  source 1..20: mol_type = other DNA
atgccggtcataacagtgtc

SEQ ID NO: 125 - DNA, 20 bp, synthetic construct
  misc_feature 1..20: note = Synthetic Polynucleotide
  source 1..20: mol_type = other DNA
tggtggctcgttatcacaag

SEQ ID NO: 126 - DNA, 20 bp, synthetic construct
  misc_feature 1..20: note = Synthetic Polynucleotide
  source 1..20: mol_type = other DNA
tctgaagcgtggccattacc

SEQ ID NO: 127 - DNA, 20 bp, synthetic construct
  misc_feature 1..20: note = Synthetic Polynucleotide
  source 1..20: mol_type = other DNA
catatgccggacattcagct

SEQ ID NO: 128 - DNA, 20 bp, synthetic construct
  misc_feature 1..20: note = Synthetic Polynucleotide
  source 1..20: mol_type = other DNA
actaatggtcctgcggcata

SEQ ID NO: 129 - DNA, 20 bp, synthetic construct
  misc_feature 1..20: note = Synthetic Polynucleotide
  source 1..20: mol_type = other DNA
ggtgccgtgttgcatgtaag

SEQ ID NO: 130 - DNA, 20 bp, synthetic construct
  misc_feature 1..20: note = Synthetic Polynucleotide
  source 1..20: mol_type = other DNA
aagcggtcgtggtttatacc

SEQ ID NO: 131 - DNA, 20 bp, synthetic construct
  misc_feature 1..20: note = Synthetic Polynucleotide
  source 1..20: mol_type = other DNA
gccaccttgtatggtatcga

SEQ ID NO: 132 - DNA, 20 bp, synthetic construct
  misc_feature 1..20: note = Synthetic Polynucleotide
  source 1..20: mol_type = other DNA
taatgcttaggcccgtcggt

SEQ ID NO: 133 - DNA, 20 bp, synthetic construct
  misc_feature 1..20: note = Synthetic Polynucleotide
  source 1..20: mol_type = other DNA
gacgcgacggattatttagg

SEQ ID NO: 134 - DNA, 20 bp, synthetic construct
  misc_feature 1..20: note = Synthetic Polynucleotide
  source 1..20: mol_type = other DNA
ggtcgcgcccatatataagg

SEQ ID NO: 135 - DNA, 20 bp, synthetic construct
  misc_feature 1..20: note = Synthetic Polynucleotide
  source 1..20: mol_type = other DNA
tgactacggttgggtgcatc

SEQ ID NO: 136 - DNA, 20 bp, synthetic construct
  misc_feature 1..20: note = Synthetic Polynucleotide
  source 1..20: mol_type = other DNA
tcaggcgctcattgtatgtc

SEQ ID NO: 137 - DNA, 20 bp, synthetic construct
  misc_feature 1..20: note = Synthetic Polynucleotide
  source 1..20: mol_type = other DNA
gtagatactcccggcccgaa

SEQ ID NO: 138 - DNA, 20 bp, synthetic construct
  misc_feature 1..20: note = Synthetic Polynucleotide
  source 1..20: mol_type = other DNA
gcagcacttagggcagcatc

SEQ ID NO: 139 - DNA, 20 bp, synthetic construct
  misc_feature 1..20: note = Synthetic Polynucleotide
  source 1..20: mol_type = other DNA
cgcgggaaccatattaggaa

SEQ ID NO: 140 - DNA, 20 bp, synthetic construct
  misc_feature 1..20: note = Synthetic Polynucleotide
  source 1..20: mol_type = other DNA
gatccatggccagttcgtat

SEQ ID NO: 141 - DNA, 20 bp, synthetic construct
  misc_feature 1..20: note = Synthetic Polynucleotide
  source 1..20: mol_type = other DNA
gtgcgccaaaggacttagtg

SEQ ID NO: 142 - DNA, 20 bp, synthetic construct
  misc_feature 1..20: note = Synthetic Polynucleotide
  source 1..20: mol_type = other DNA
gatcccgctattcaccgatt

SEQ ID NO: 143 - DNA, 20 bp, synthetic construct
  misc_feature 1..20: note = Synthetic Polynucleotide
  source 1..20: mol_type = other DNA
gcatagatggttcacccgta

SEQ ID NO: 144 - DNA, 20 bp, synthetic construct
  misc_feature 1..20: note = Synthetic Polynucleotide
  source 1..20: mol_type = other DNA
gtctataagccgcgctgcaa

SEQ ID NO: 145 - DNA, 20 bp, synthetic construct
  misc_feature 1..20: note = Synthetic Polynucleotide
  source 1..20: mol_type = other DNA
gcggagtatgccatcatgag

SEQ ID NO: 146 - DNA, 20 bp, synthetic construct
  misc_feature 1..20: note = Synthetic Polynucleotide
  source 1..20: mol_type = other DNA
tgccgcgatcatctactatg

SEQ ID NO: 147 - DNA, 20 bp, synthetic construct
  misc_feature 1..20: note = Synthetic Polynucleotide
  source 1..20: mol_type = other DNA
gaccgggtattcgacgtcat

SEQ ID NO: 148 - DNA, 20 bp, synthetic construct
  misc_feature 1..20: note = Synthetic Polynucleotide
  source 1..20: mol_type = other DNA
gcagtgcgggtagatacgct

SEQ ID NO: 149 - DNA, 20 bp, synthetic construct
  misc_feature 1..20: note = Synthetic Polynucleotide
  source 1..20: mol_type = other DNA
aggcgtgggtagcaacgtat

SEQ ID NO: 150 - DNA, 20 bp, synthetic construct
  misc_feature 1..20: note = Synthetic Polynucleotide
  source 1..20: mol_type = other DNA
gctcattggactttctccca

SEQ ID NO: 151 - DNA, 20 bp, synthetic construct
  misc_feature 1..20: note = Synthetic Polynucleotide
  source 1..20: mol_type = other DNA
aaggaccacgtatctgcatg

SEQ ID NO: 152 - DNA, 20 bp, synthetic construct
  misc_feature 1..20: note = Synthetic Polynucleotide
  source 1..20: mol_type = other DNA
atctgatagtgccgcgacgt

SEQ ID NO: 153 - DNA, 20 bp, synthetic construct
  misc_feature 1..20: note = Synthetic Polynucleotide
  source 1..20: mol_type = other DNA
tgacctgcacggatagtagg

SEQ ID NO: 154 - DNA, 20 bp, synthetic construct
  misc_feature 1..20: note = Synthetic Polynucleotide
  source 1..20: mol_type = other DNA
gttattcgcagtccttgggt

SEQ ID NO: 155 - DNA, 20 bp, synthetic construct
  misc_feature 1..20: note = Synthetic Polynucleotide
  source 1..20: mol_type = other DNA
gtgcagtgtgcttaaatccg

SEQ ID NO: 156 - DNA, 20 bp, synthetic construct
  misc_feature 1..20: note = Synthetic Polynucleotide
  source 1..20: mol_type = other DNA
cgcgggtgttaaataaggac

SEQ ID NO: 157 - DNA, 20 bp, synthetic construct
  misc_feature 1..20: note = Synthetic Polynucleotide
  source 1..20: mol_type = other DNA
gcgaggcgtggtaatagtca

SEQ ID NO: 158 - DNA, 20 bp, synthetic construct
  misc_feature 1..20: note = Synthetic Polynucleotide
  source 1..20: mol_type = other DNA
caaggcgcaaacatagacag

SEQ ID NO: 159 - DNA, 20 bp, synthetic construct
  misc_feature 1..20: note = Synthetic Polynucleotide
  source 1..20: mol_type = other DNA
ggaacaagggcgtctatgtc

SEQ ID NO: 160 - DNA, 20 bp, synthetic construct
  misc_feature 1..20: note = Synthetic Polynucleotide
  source 1..20: mol_type = other DNA
gcagtgcggataagctacac

SEQ ID NO: 161 - DNA, 20 bp, synthetic construct
  misc_feature 1..20: note = Synthetic Polynucleotide
  source 1..20: mol_type = other DNA
agagatgcgtgtaggcgatt

SEQ ID NO: 162 - DNA, 20 bp, synthetic construct
  misc_feature 1..20: note = Synthetic Polynucleotide
  source 1..20: mol_type = other DNA
ccctttcgagctaagtttgg

SEQ ID NO: 163 - DNA, 20 bp, synthetic construct
  misc_feature 1..20: note = Synthetic Polynucleotide
  source 1..20: mol_type = other DNA
ccttaagcaacccgtcgatg

SEQ ID NO: 164 - DNA, 20 bp, synthetic construct
  misc_feature 1..20: note = Synthetic Polynucleotide
  source 1..20: mol_type = other DNA
ttgctcgtgactgaacaacg

SEQ ID NO: 165 - DNA, 20 bp, synthetic construct
  misc_feature 1..20: note = Synthetic Polynucleotide
  source 1..20: mol_type = other DNA
ggtcgtgcgtataagcctca

SEQ ID NO: 166 - DNA, 20 bp, synthetic construct
  misc_feature 1..20: note = Synthetic Polynucleotide
  source 1..20: mol_type = other DNA
cggatggtcttcgtttaacc

SEQ ID NO: 167 - DNA, 20 bp, synthetic construct
  misc_feature 1..20: note = Synthetic Polynucleotide
  source 1..20: mol_type = other DNA
ggcacggtggctagtaacga

SEQ ID NO: 168 - DNA, 20 bp, synthetic construct
  misc_feature 1..20: note = Synthetic Polynucleotide
  source 1..20: mol_type = other DNA
gccggcccaactgatagtag

SEQ ID NO: 169 - DNA, 20 bp, synthetic construct
  misc_feature 1..20: note = Synthetic Polynucleotide
  source 1..20: mol_type = other DNA
tcgacttcggtcacctttcc

SEQ ID NO: 170 - DNA, 20 bp, synthetic construct
  misc_feature 1..20: note = Synthetic Polynucleotide
  source 1..20: mol_type = other DNA
cgggtcgatactttcctcgt

SEQ ID NO: 171 - DNA, 20 bp, synthetic construct
  misc_feature 1..20: note = Synthetic Polynucleotide
  source 1..20: mol_type = other DNA
gtgccgtgtgtaatatccga

SEQ ID NO: 172 - DNA, 20 bp, synthetic construct
  misc_feature 1..20: note = Synthetic Polynucleotide
  source 1..20: mol_type = other DNA
cgcgcctggaataactccta

SEQ ID NO: 173 - DNA, 20 bp, synthetic construct
  misc_feature 1..20: note = Synthetic Polynucleotide
  source 1..20: mol_type = other DNA
gtcagggaaccgtttcttca

SEQ ID NO: 174 - DNA, 20 bp, synthetic construct
  misc_feature 1..20: note = Synthetic Polynucleotide
  source 1..20: mol_type = other DNA
cttgtcatgtacccgaatgg

SEQ ID NO: 175 - DNA, 20 bp, synthetic construct
  misc_feature 1..20: note = Synthetic Polynucleotide
  source 1..20: mol_type = other DNA
cgacctggctacgtagaacc

SEQ ID NO: 176 - DNA, 20 bp, synthetic construct
  misc_feature 1..20: note = Synthetic Polynucleotide
  source 1..20: mol_type = other DNA
ctagctaaagggccgtgcgt

SEQ ID NO: 177 - DNA, 20 bp, synthetic construct
  misc_feature 1..20: note = Synthetic Polynucleotide
  source 1..20: mol_type = other DNA
aagtattagcgcggcaacgt

SEQ ID NO: 178 - DNA, 20 bp, synthetic construct
  misc_feature 1..20: note = Synthetic Polynucleotide
  source 1..20: mol_type = other DNA
ggttgcgtgccacttaaagc

SEQ ID NO: 179 - DNA, 20 bp, synthetic construct
  misc_feature 1..20: note = Synthetic Polynucleotide
  source 1..20: mol_type = other DNA
ggaggttcggttgtactgca

SEQ ID NO: 180 - DNA, 20 bp, synthetic construct
  misc_feature 1..20: note = Synthetic Polynucleotide
  source 1..20: mol_type = other DNA
gttgtcgtcctccatcggtt

SEQ ID NO: 181 - DNA, 20 bp, synthetic construct
  misc_feature 1..20: note = Synthetic Polynucleotide
  source 1..20: mol_type = other DNA
gggacggaactacacatgtg

SEQ ID NO: 182 - DNA, 20 bp, synthetic construct
  misc_feature 1..20: note = Synthetic Polynucleotide
  source 1..20: mol_type = other DNA
gatccatcctgattgagggt

SEQ ID NO: 183 - DNA, 20 bp, synthetic construct
  misc_feature 1..20: note = Synthetic Polynucleotide
  source 1..20: mol_type = other DNA
cttcatggtaccggttgaga

SEQ ID NO: 184 - DNA, 20 bp, synthetic construct
  misc_feature 1..20: note = Synthetic Polynucleotide
  source 1..20: mol_type = other DNA
tcggtcggctgtaaggatac

SEQ ID NO: 185 - DNA, 20 bp, synthetic construct
  misc_feature 1..20: note = Synthetic Polynucleotide
  source 1..20: mol_type = other DNA
attgcacgaggtcagagtcg

SEQ ID NO: 186 - DNA, 20 bp, synthetic construct
  misc_feature 1..20: note = Synthetic Polynucleotide
  source 1..20: mol_type = other DNA
gcttagatccgctcgctacg

SEQ ID NO: 187 - DNA, 20 bp, synthetic construct
  misc_feature 1..20: note = Synthetic Polynucleotide
  source 1..20: mol_type = other DNA
cggctggcctactgtagaga

SEQ ID NO: 188 - DNA, 20 bp, synthetic construct
  misc_feature 1..20: note = Synthetic Polynucleotide
  source 1..20: mol_type = other DNA
gcaacatgacctgtcatcgc

SEQ ID NO: 189 - DNA, 20 bp, synthetic construct
  misc_feature 1..20: note = Synthetic Polynucleotide
  source 1..20: mol_type = other DNA
ggccgcacgatatatttgac

SEQ ID NO: 190 - DNA, 20 bp, synthetic construct
  misc_feature 1..20: note = Synthetic Polynucleotide
  source 1..20: mol_type = other DNA
cagccgggccataatagttg

SEQ ID NO: 191 - DNA, 20 bp, synthetic construct
  misc_feature 1..20: note = Synthetic Polynucleotide
  source 1..20: mol_type = other DNA
tgcggtggacctattatcct

SEQ ID NO: 192 - DNA, 20 bp, synthetic construct
  misc_feature 1..20: note = Synthetic Polynucleotide
  source 1..20: mol_type = other DNA
cctgcgttaggcaatccatc

SEQ ID NO: 193 - DNA, 20 bp, synthetic construct
  misc_feature 1..20: note = Synthetic Polynucleotide
  source 1..20: mol_type = other DNA
gtgggctcttcgaagtaacc

SEQ ID NO: 194 - DNA, 20 bp, synthetic construct
  misc_feature 1..20: note = Synthetic Polynucleotide
  source 1..20: mol_type = other DNA
gccgtggaccactaaagttc

SEQ ID NO: 195 - DNA, 20 bp, synthetic construct
  misc_feature 1..20: note = Synthetic Polynucleotide
  source 1..20: mol_type = other DNA
gcttaagtcatgggcgcatc

SEQ ID NO: 196 - DNA, 20 bp, synthetic construct
  misc_feature 1..20: note = Synthetic Polynucleotide
  source 1..20: mol_type = other DNA
acaggttagttccgcgcact

SEQ ID NO: 197 - DNA, 20 bp, synthetic construct
  misc_feature 1..20: note = Synthetic Polynucleotide
  source 1..20: mol_type = other DNA
ggcgtggcatttagactacc

SEQ ID NO: 198 - DNA, 20 bp, synthetic construct
  misc_feature 1..20: note = Synthetic Polynucleotide
  source 1..20: mol_type = other DNA
actacggccaacaaccaaca

SEQ ID NO: 199 - DNA, 20 bp, synthetic construct
  misc_feature 1..20: note = Synthetic Polynucleotide
  source 1..20: mol_type = other DNA
gttgacaaggctctgtacgg

SEQ ID NO: 200 - DNA, 20 bp, synthetic construct
  misc_feature 1..20: note = Synthetic Polynucleotide
  source 1..20: mol_type = other DNA
ttgttctcttgccggtcgat

SEQ ID NO: 201 - DNA, 20 bp, synthetic construct
  misc_feature 1..20: note = Synthetic Polynucleotide
  source 1..20: mol_type = other DNA
gttgatcgtagccaattcgg

SEQ ID NO: 202 - DNA, 102 bp, synthetic construct; identical to SEQ ID NO: 3
  misc_feature 1..102: note = Synthetic Polynucleotide
  source 1..102: mol_type = other DNA

SEQ ID NO: 203 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctttcgcgatagcagccaggaagcctgcttcattgcgcttctccggaaacattagc

SEQ ID NO: 204 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccagcgggaaacctttctgttgcagagcttcattgcgcttctccggaaacattagc

SEQ ID NO: 205 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgccagttccgcagaagccaggaagacattcattgcgcttctccggaaacattagc

SEQ ID NO: 206 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggtgtggcagaaagactgcatcaccaggtcattgcgcttctccggaaacattagc

SEQ ID NO: 207 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgccgcgtttgttcggcacatgtggaatatcattgcgcttctccggaaacattagc

SEQ ID NO: 208 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggtgcgtgttcacgtcaactggcttcggatcattgcgcttctccggaaacattagc

SEQ ID NO: 209 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccatctgcatcggcttccagcagctcaggatcattgcgcttctccggaaacattagc

SEQ ID NO: 210 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcagcacggaagtgaccgatgttggtcatcattgcgcttctccggaaacattagc

SEQ ID NO: 211 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccatccgggtcgttcggagcacacaggattgcattgcgcttctccggaaacattagc

SEQ ID NO: 212 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatacgcgcaccactcttaccgaagacgctgcattgcgcttctccggaaacattagc

SEQ ID NO: 213 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatcggctttctcggtgtactgagaaagctgcattgcgcttctccggaaacattagc

SEQ ID NO: 214 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagtacccagacggttcgggaagttacgggcattgcgcttctccggaaacattagc

SEQ ID NO: 215 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctttgttcagcttgatggtacaaccagcggcattgcgcttctccggaaacattagc

SEQ ID NO: 216 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccatccgcccaggactgcataacctgcttcgcattgcgcttctccggaaacattagc

SEQ ID NO: 217 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccccataaaccacagaacggagttagtggcgcattgcgcttctccggaaacattagc

SEQ ID NO: 218 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcgttgcctgctttcgctttctcttctacgcattgcgcttctccggaaacattagc

SEQ ID NO: 219 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccggtgccggagaaaggtcatcggtgttagcattgcgcttctccggaaacattagc

SEQ ID NO: 220 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccgccacggttcataatgaagtccggcagcattgcgcttctccggaaacattagc

SEQ ID NO: 221 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggtcggcaactgacctttatgcgcatccagcattgcgcttctccggaaacattagc

SEQ ID NO: 222 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagttcgccggtttcgtggttacgcacttccattgcgcttctccggaaacattagc

SEQ ID NO: 223 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcgccgtaaccttccgcgatcatccacttccattgcgcttctccggaaacattagc

SEQ ID NO: 224 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccttgatcggaccaacaacaccaggctggtccattgcgcttctccggaaacattagc

SEQ ID NO: 225 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccggtggtgtcctgggaacctacagaagtccattgcgcttctccggaaacattagc

SEQ ID NO: 226 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggacgcatcggttagctcaaaggcctgctccattgcgcttctccggaaacattagc

SEQ ID NO: 227 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatctacctgcgccacgtaggtctggtactccattgcgcttctccggaaacattagc

SEQ ID NO: 228 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatacggccaccagcacgcacttcatcaatccattgcgcttctccggaaacattagc

SEQ ID NO: 229 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatcgaacatcagcagcgtgtgagaaagtgccattgcgcttctccggaaacattagc

SEQ ID NO: 230 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaatatccgccagatcgatgtcgatcactgccattgcgcttctccggaaacattagc

SEQ ID NO: 231 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaacacttcgtcgatcttctcaccctgtacccattgcgcttctccggaaacattagc

SEQ ID NO: 232 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccttgcctttcttctcaacggtcagcagacccattgcgcttctccggaaacattagc

SEQ ID NO: 233 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccatgccctgaatacgacgttccagggtaccattgcgcttctccggaaacattagc

SEQ ID NO: 234 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggaaacgggtatgggagtcaccaccggtaccattgcgcttctccggaaacattagc

SEQ ID NO: 235 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgaagaacccgtacccacaacgtcaccgaccattgcgcttctccggaaacattagc

SEQ ID NO: 236 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgtcaatcacgtcgcccatgttcaggttacattgcgcttctccggaaacattagc

SEQ ID NO: 237 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccagaccagaacccgccgggaaagagatacattgcgcttctccggaaacattagc

SEQ ID NO: 238 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttgaagcgcaccagaacggattccggcatacattgcgcttctccggaaacattagc

SEQ ID NO: 239 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccttcttcggtcaactgtgcggcgtccatacattgcgcttctccggaaacattagc

SEQ ID NO: 240 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggttacccatacacagggaacagccagggacattgcgcttctccggaaacattagc

SEQ ID NO: 241 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcagatccggcagaccttcaatttccaggacattgcgcttctccggaaacattagc

SEQ ID NO: 242 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccagtttggcatcatccagcgcgtcgatcacattgcgcttctccggaaacattagc

SEQ ID NO: 243 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccccaggcacgccaggtctttcagttcatcacattgcgcttctccggaaacattagc

SEQ ID NO: 244 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctttggtggtcaggccacgcccgataatcacattgcgcttctccggaaacattagc

SEQ ID NO: 245 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgatcgcatacagcggaatagcgtgtaccacattgcgcttctccggaaacattagc

SEQ ID NO: 246 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcagtgcacccgcgtcttccatcgtgttaacattgcgcttctccggaaacattagc

SEQ ID NO: 247 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcagcagcatacggttcagccaggagtgaacattgcgcttctccggaaacattagc

SEQ ID NO: 248 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgctctcagcgacatctttcgcctgacggaacattgcgcttctccggaaacattagc

SEQ ID NO: 249 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggtttcaaggtcgcccgccagtttctgtttcattgcgcttctccggaaacattagc

SEQ ID NO: 250 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccacggtgctggaaccaccggcatagaagttcattgcgcttctccggaaacattagc

SEQ ID NO: 251 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggtcccgttgataccaacagcataacctgtcattgcgcttctccggaaacattagc

SEQ ID NO: 252 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgaaacgggcataaccgcgatccagatagtcattgcgcttctccggaaacattagc

SEQ ID NO: 253 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggaacacgttgggtatcggtatcgacagtcattgcgcttctccggaaacattagc

SEQ ID NO: 254 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacggtgaagtacgggttggttaccgacagtcattgcgcttctccggaaacattagc

SEQ ID NO: 255 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacagctcacccggctcgatcttagtcagctcattgcgcttctccggaaacattagc

SEQ ID NO: 256 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgttggtatagtcggacaggtcggcgtcatcattgcgcttctccggaaacattagc

SEQ ID NO: 257 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggcgacacgctgaaggccttcgaaatgaatcattgcgcttctccggaaacattagc

SEQ ID NO: 258 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctggaaatgagagatcagttcgtcggtggtgcattgcgcttctccggaaacattagc

SEQ ID NO: 259 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttgctggatttcagctgacacaccttcctgcattgcgcttctccggaaacattagc

SEQ ID NO: 260 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtcggcatcgttaatttcgggcatcgactgcattgcgcttctccggaaacattagc

SEQ ID NO: 261 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcactgctttcggaccaatggtattggactggcattgcgcttctccggaaacattagc

SEQ ID NO: 262 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccttgtagacgacatcaacctggtccgggcattgcgcttctccggaaacattagc

SEQ ID NO: 263 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctatccgatccaggaatggtcactttaccggcattgcgcttctccggaaacattagc

SEQ ID NO: 264 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcactacgaacccttcagcaccgtatacggcattgcgcttctccggaaacattagc

SEQ ID NO: 265 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagccatgcaccttccatctgacgcatttcgcattgcgcttctccggaaacattagc

SEQ ID NO: 266 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccccagacctgcacgcagcgagttatattcgcattgcgcttctccggaaacattagc

SEQ ID NO: 267 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaactggaactgttctgccttgtctccatcgcattgcgcttctccggaaacattagc

SEQ ID NO: 268 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatcttcatcattaaccgtgtcgcctgtgcgcattgcgcttctccggaaacattagc

SEQ ID NO: 269 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctccaaccatagttgaacgtgaagtcgtccgcattgcgcttctccggaaacattagc

SEQ ID NO: 270 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccattgtaatgcgatacccgcagacatacgcattgcgcttctccggaaacattagc

SEQ ID NO: 271 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatttacgatcgcctaccacgttccaccacgcattgcgcttctccggaaacattagc

SEQ ID NO: 272 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctggctggaatcccagtttgtatcccaaacgcattgcgcttctccggaaacattagc

SEQ ID NO: 273 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgctggcgctatatttaccgacgctgtagcattgcgcttctccggaaacattagc

SEQ ID NO: 274 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccgcctacagcatcatccgatttacacagcattgcgcttctccggaaacattagc

SEQ ID NO: 275 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatctggcgtcagactgacctgggtagagtccattgcgcttctccggaaacattagc

SEQ ID NO: 276 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcgccgtcctgagtcgcacattcgtaatccattgcgcttctccggaaacattagc

SEQ ID NO: 277 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacaacccatttgtgatcgtcatcgatcggccattgcgcttctccggaaacattagc

SEQ ID NO: 278 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacccatagagtacagataacgccacatcgccattgcgcttctccggaaacattagc

SEQ ID NO: 279 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcataccaaagttgaagctaccggtgttgcgccattgcgcttctccggaaacattagc

SEQ ID NO: 280 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctatagaagagacgaccaccgaggcttacgccattgcgcttctccggaaacattagc

SEQ ID NO: 281 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtcatcttccatcttggtcactttggtgcccattgcgcttctccggaaacattagc

SEQ ID NO: 282 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggatcttacgcacgtagaaacggttaccccattgcgcttctccggaaacattagc

SEQ ID NO: 283 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaggacggcatctttcgaggtatcgttacccattgcgcttctccggaaacattagc

SEQ ID NO: 284 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagaagcccagacgattcagacgctccttaccattgcgcttctccggaaacattagc

SEQ ID NO: 285 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgacacgtgaaccatctgtcgggaagtaaccattgcgcttctccggaaacattagc

SEQ ID NO: 286 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgctaaacagcagcgacgctatgagcaaccattgcgcttctccggaaacattagc

SEQ ID NO: 287 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgaggctgcatgttggacagggagttatgtacattgcgcttctccggaaacattagc

SEQ ID NO: 288 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgggaagcccaacgtcacgtctgtaccatacattgcgcttctccggaaacattagc

SEQ ID NO: 289 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccggatcataattactggcctgatgcgggacattgcgcttctccggaaacattagc

SEQ ID NO: 290 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaagctgttatcctgatcagaggtgctcggacattgcgcttctccggaaacattagc

SEQ ID NO: 291 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacctgaaccagaagggtatcaccatcacgacattgcgcttctccggaaacattagc

SEQ ID NO: 292 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggcatctctttgccgcctaaaccatcaccacattgcgcttctccggaaacattagc

SEQ ID NO: 293 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccagttatcctgctgcacaccagcctggaacattgcgcttctccggaaacattagc

SEQ ID NO: 294 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcggataggcataaccatagcgaccgagaacattgcgcttctccggaaacattagc

SEQ ID NO: 295 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaaggttgccgctcacttcaacgccagaaacattgcgcttctccggaaacattagc

SEQ ID NO: 296 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgaacatcctcaaagttgccggtagcaaacattgcgcttctccggaaacattagc

SEQ ID NO: 297 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgtgggcttgcacgttcaaaccagtcactcattgcgcttctccggaaacattagc

SEQ ID NO: 298 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccctgtgaaagcgcattacagagctgttgtgcattgcgcttctccggaaacattagc

SEQ ID NO: 299 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttgcgctttcaattgcgcgtactgatccagcattgcgcttctccggaaacattagc

SEQ ID NO: 300 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgcttgtggaggatagtcggaatagctgccattgcgcttctccggaaacattagc

SEQ ID NO: 301 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaggaatttggtccggtccgcctgtaatgaccattgcgcttctccggaaacattagc

SEQ ID NO: 302 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgagcgtgctaacacctgttcgcggctaaccattgcgcttctccggaaacattagc

SEQ ID NO: 303 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcattacctccacgccaggcaatcaccagacattgcgcttctccggaaacattagc

SEQ ID NO: 304 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcaaaggcaagttcagtgttggcgggagaacattgcgcttctccggaaacattagc

SEQ ID NO: 305 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgatatcgcggatatgcagcagccagttgttcattgcgcttctccggaaacattagc

SEQ ID NO: 306 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcagttcaggtcagtgtgaatgaccaggttcattgcgcttctccggaaacattagc

SEQ ID NO: 307 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccattacggtgaacaaagagttcgcccggctcattgcgcttctccggaaacattagc

SEQ ID NO: 308 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttgtggttggcgtgtttcagcttgaggttgcattgcgcttctccggaaacattagc

SEQ ID NO: 309 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccggttaaacgttctgcaggaacgcgactgcattgcgcttctccggaaacattagc

SEQ ID NO: 310 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccctgttccatgacgttcagttcacacaaggcattgcgcttctccggaaacattagc

SEQ ID NO: 311 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcgagtacatccactgcatactgaaccacgcattgcgcttctccggaaacattagc

SEQ ID NO: 312 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgtttccacgctgattgcataatggtggagcattgcgcttctccggaaacattagc

SEQ ID NO: 313 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccccgtgacggtaacgttgctcaagggtttccattgcgcttctccggaaacattagc

SEQ ID NO: 314 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgccgcaaccgtagtggccacagataatacattgcgcttctccggaaacattagc

SEQ ID NO: 315 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggtggcggtaacatccagatcacgcagcaacattgcgcttctccggaaacattagc

SEQ ID NO: 316 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctaatgattctcgtcatagtgccaggcccgtcattgcgcttctccggaaacattagc

SEQ ID NO: 317 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagaaacaacgccgcgatgacgacgggtatcattgcgcttctccggaaacattagc

SEQ ID NO: 318 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccagcatgcagaccaccagcgttacaatgcattgcgcttctccggaaacattagc

SEQ ID NO: 319 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagagttacgccagattgctgcggaacctggcattgcgcttctccggaaacattagc

SEQ ID NO: 320 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgaagcccaaagtgagatttcaaccagcggcattgcgcttctccggaaacattagc

SEQ ID NO: 321 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtaacaacaagcaagcaaagaccggtaccgcattgcgcttctccggaaacattagc

SEQ ID NO: 322 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggatcatgacatagagcagccatattgcgccattgcgcttctccggaaacattagc

SEQ ID NO: 323 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctaccaccgtgtcctggaattaaatgaccgccattgcgcttctccggaaacattagc

SEQ ID NO: 324 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctttgccaaacatatatgcgccggagtcagccattgcgcttctccggaaacattagc

SEQ ID NO: 325 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgctgcagtagcgagtccaccgataaagccattgcgcttctccggaaacattagc

SEQ ID NO: 326 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgcttaaacatactctcggtcagatcgcccattgcgcttctccggaaacattagc

SEQ ID NO: 327 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcatcagcgccaacaataacccgcataacacattgcgcttctccggaaacattagc

SEQ ID NO: 328 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgacgtcgagattcgcccacatgccataacattgcgcttctccggaaacattagc

SEQ ID NO: 329 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagcaccagcaatagcgcgacaatccaccaacattgcgcttctccggaaacattagc

SEQ ID NO: 330 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgaggctaacgctgcgacaatagagcaaacattgcgcttctccggaaacattagc

SEQ ID NO: 331 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgaacgcgaccgtagtggatttcgccggtttcattgcgcttctccggaaacattagc

SEQ ID NO: 332 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccggaatccagcagggcgatcacctgatttcattgcgcttctccggaaacattagc

SEQ ID NO: 333 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtgccttcatcaacatatgcgccgatgttgcattgcgcttctccggaaacattagc

SEQ ID NO: 334 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaaccactgatgcgtcacccactgaccgtccattgcgcttctccggaaacattagc

SEQ ID NO: 335 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtaacggtgtctgcattggctggcgtgatccattgcgcttctccggaaacattagc

SEQ ID NO: 336 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaaccgcacagtagaggctgtatttgccatccattgcgcttctccggaaacattagc

SEQ ID NO: 337 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagtcgatggtacgcagcagttcgttaatgccattgcgcttctccggaaacattagc

SEQ ID NO: 338 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaatacgggtgctctgaccaatgtatacgcccattgcgcttctccggaaacattagc

SEQ ID NO: 339 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacttcagagcgcgcgccgatgaagcaattacattgcgcttctccggaaacattagc

SEQ ID NO: 340 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgacggcatcagcacggtgttacgggcaatacattgcgcttctccggaaacattagc

SEQ ID NO: 341 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccttcgtcgtagtcggcgaatttcatcggcacattgcgcttctccggaaacattagc

SEQ ID NO: 342 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctggcacaacgcggaagccttctttctggaacattgcgcttctccggaaacattagc

SEQ ID NO: 343 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccacaagaaccgacggtcgcccaggtatcaacattgcgcttctccggaaacattagc

SEQ ID NO: 344 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccggtaaaccggcaccttgccatagaagttcattgcgcttctccggaaacattagc

SEQ ID NO: 345 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggtcgacaggatacgcgtcaataccgccttcattgcgcttctccggaaacattagc

SEQ ID NO: 346 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgctgtggctggtcatacccggtgaggtattcattgcgcttctccggaaacattagc

SEQ ID NO: 347 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgtccatcgctgtccagcataacgtcaatcattgcgcttctccggaaacattagc

SEQ ID NO: 348 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcgcctcataatcatagaaggttccggacgcattgcgcttctccggaaacattagc

SEQ ID NO: 349 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccctttgcaacctaacgtcgtccatgctttccattgcgcttctccggaaacattagc

SEQ ID NO: 350 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgcgaccgtgtagcgcgataaacactttccattgcgcttctccggaaacattagc

SEQ ID NO: 351 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgacattcccacactggaaccttcgcggctccattgcgcttctccggaaacattagc

SEQ ID NO: 352 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttcttcaccgagtatcgcaaccgtgaactccattgcgcttctccggaaacattagc

SEQ ID NO: 353 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaaccggcaaacccagagcagaaatttctgccattgcgcttctccggaaacattagc

SEQ ID NO: 354 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctaatgcctgcaaattggcctcttgtgacgccattgcgcttctccggaaacattagc

SEQ ID NO: 355 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatccattgaaagcgcagatgccatcactcccattgcgcttctccggaaacattagc

SEQ ID NO: 356 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctagtccgccagttccagaattcgtactacccattgcgcttctccggaaacattagc

SEQ ID NO: 357 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcccatcgacttcagttgcgtcacgtcgaccattgcgcttctccggaaacattagc

SEQ ID NO: 358 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggctaacaccgctgcgccagaattcagagacattgcgcttctccggaaacattagc

SEQ ID NO: 359 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggtataaggcaagcccatcagctcgagcacattgcgcttctccggaaacattagc

SEQ ID NO: 360 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccacgttgttcactaccacgccctgatctttcattgcgcttctccggaaacattagc

SEQ ID NO: 361 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgttcaggttaaccagcgcaccaccggagttcattgcgcttctccggaaacattagc

SEQ ID NO: 362 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgcagttccaggttcacgttaacctgcttcattgcgcttctccggaaacattagc

SEQ ID NO: 363 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcagcggaggaattaggcagaacctggcttcattgcgcttctccggaaacattagc

SEQ ID NO: 364 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcactcagagccagtgcactcagtgctaatgtcattgcgcttctccggaaacattagc

SEQ ID NO: 365 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgttgctcatctcagcgccttcaatgccgtcattgcgcttctccggaaacattagc

SEQ ID NO: 366 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgaatgttgagtgccagcacagacggtttgcattgcgcttctccggaaacattagc

SEQ ID NO: 367 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccggaacccagcgccatgaatttctgttgcattgcgcttctccggaaacattagc

SEQ ID NO: 368 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatggagctggaatcaacctgattctggctgcattgcgcttctccggaaacattagc

SEQ ID NO: 369 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcataatacccagctcaccgcgtttcacctggcattgcgcttctccggaaacattagc

SEQ ID NO: 370 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggcaaagctgctgatcggcttaccgttcagcattgcgcttctccggaaacattagc

SEQ ID NO: 371 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgggcgtcaactttcatcgctttcgccagcattgcgcttctccggaaacattagc

SEQ ID NO: 372 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcatctgctgggctgtcgttgctgaagaagcattgcgcttctccggaaacattagc

SEQ ID NO: 373 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagcgcagagacaatcccggaagttaccgtccattgcgcttctccggaaacattagc

SEQ ID NO: 374 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgggttctggatttggatcagcgcgatatccattgcgcttctccggaaacattagc

SEQ ID NO: 375 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggtgcgaggatcgcggtgttgataccgatccattgcgcttctccggaaacattagc

SEQ ID NO: 376 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcactgcattaacaggtagatggtgctgtcgccattgcgcttctccggaaacattagc

SEQ ID NO: 377 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaacaacgtggttgttggtgacgacatagccattgcgcttctccggaaacattagc

SEQ ID NO: 378 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggccatcgctcagttgaactttaatgacccattgcgcttctccggaaacattagc

SEQ ID NO: 379 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgttaatgctgaccactgaaggcatcacccattgcgcttctccggaaacattagc

SEQ ID NO: 380 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccactgcctgctggttcgcgccaataatcaccattgcgcttctccggaaacattagc

SEQ ID NO: 381 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgcgcagtgcatcagaatccgccatcttacattgcgcttctccggaaacattagc

SEQ ID NO: 382 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccaaacgggttaccaatcgctacggtgtacattgcgcttctccggaaacattagc

SEQ ID NO: 383 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggcatacgcggcgtattaacggttgtgctacattgcgcttctccggaaacattagc

SEQ ID NO: 384 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcagtaagcccagggtcagtttgctgcctacattgcgcttctccggaaacattagc

SEQ ID NO: 385 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcggatctttgccaaccatcttcgcgtcgacattgcgcttctccggaaacattagc

SEQ ID NO: 386 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctggcagaacggagagctctggaacggagaacattgcgcttctccggaaacattagc

SEQ ID NO: 387 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctggcagaacggagaatcatcaccgaagaacattgcgcttctccggaaacattagc

SEQ ID NO: 388 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccgttgcagagagcggagataacgccaaacattgcgcttctccggaaacattagc

SEQ ID NO: 389 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgatggtcatatcgccttcgttcacctgatcattgcgcttctccggaaacattagc

SEQ ID NO: 390 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagattcgggttatcttcgtcagcgatcaggcattgcgcttctccggaaacattagc

SEQ ID NO: 391 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagatactaaaggaagaaccgcaaccgcaggcattgcgcttctccggaaacattagc

SEQ ID NO: 392 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaactttgttggctgctgcgtcggtaaactccattgcgcttctccggaaacattagc

SEQ ID NO: 393 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccttcggtataatcaacggaaccgccgaccattgcgcttctccggaaacattagc

SEQ ID NO: 394 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcgttcgggttggtcacgatgaaacgagacattgcgcttctccggaaacattagc

SEQ ID NO: 395 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaaggtgaaaccatactggaagccgctgcaacattgcgcttctccggaaacattagc

SEQ ID NO: 396 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccctgcaacttcaggccattcagatagttcattgcgcttctccggaaacattagc

SEQ ID NO: 397 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagtctggaacaggctgtcagtaccggcttcattgcgcttctccggaaacattagc

SEQ ID NO: 398 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgctgaccatattcaacaggccgccaggactcattgcgcttctccggaaacattagc

SEQ ID NO: 399 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaccaccacgccaggtaaactgtttgtcatcattgcgcttctccggaaacattagc

SEQ ID NO: 400 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgtcgtaaccaaaccaggcgttgatgtcatcattgcgcttctccggaaacattagc

SEQ ID NO: 401 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtaaagcacggaaaccgggccacgcataatcattgcgcttctccggaaacattagc

SEQ ID NO: 402 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggtagcagactgtcgcgccgcaatagttgcattgcgcttctccggaaacattagc

SEQ ID NO: 403 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgtcccggcaacgcggttaagagattcttgcattgcgcttctccggaaacattagc

SEQ ID NO: 404 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgctttcgcttcgatttctacgccacgtgcattgcgcttctccggaaacattagc

SEQ ID NO: 405 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtcgatatcgccagtggcaaacttgctctgcattgcgcttctccggaaacattagc

SEQ ID NO: 406 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctacgtgccagataatggcctttatccgctgcattgcgcttctccggaaacattagc

SEQ ID NO: 407 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcggagcagacgccgtaaccataaacgctgcattgcgcttctccggaaacattagc

SEQ ID NO: 408 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaggtagtatcggtggtgtattccgcatcggcattgcgcttctccggaaacattagc

SEQ ID NO: 409 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatacgcataaagtcgacaccggtcagcaggcattgcgcttctccggaaacattagc

SEQ ID NO: 410 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgacttcggctgatgcagcgccatctcttcgcattgcgcttctccggaaacattagc

SEQ ID NO: 411 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccggatctttggcattgaagtcgaaatcgcattgcgcttctccggaaacattagc

SEQ ID NO: 412 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccacgatcgaacaggttgttaacatgcagcgcattgcgcttctccggaaacattagc

SEQ ID NO: 413 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcattaaagtctgtcggcagacgcttaccgcattgcgcttctccggaaacattagc

SEQ ID NO: 414 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcgttccagcatatacgggtcaatgaccgcattgcgcttctccggaaacattagc

SEQ ID NO: 415 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatacacggcaccagtaactacaatcggacgcattgcgcttctccggaaacattagc

SEQ ID NO: 416 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccagtcataacgaccgcctagggtgaccagcattgcgcttctccggaaacattagc

SEQ ID NO: 417 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccatccttcccaacttgcgaagaaggttccattgcgcttctccggaaacattagc

SEQ ID NO: 418 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcaggtgcagcggtaacggtgatagtgtccattgcgcttctccggaaacattagc

SEQ ID NO: 419 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaattcgtgatcgaagctgtagccgaccatccattgcgcttctccggaaacattagc

SEQ ID NO: 420 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgaagcagccataagtgttaaagcagctggccattgcgcttctccggaaacattagc

SEQ ID NO: 421 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcccactttaaaggagttagccggatcacccattgcgcttctccggaaacattagc

SEQ ID NO: 422 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttagaaacggaaggttgcggttgcaacgaccattgcgcttctccggaaacattagc

SEQ ID NO: 423 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaccggattgtacagattgagcagtggcaccattgcgcttctccggaaacattagc

SEQ ID NO: 424 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccaggtgaacgccggtgcaatagcataaccattgcgcttctccggaaacattagc

SEQ ID NO: 425 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcaagaccggtcaggcgataagagtaaaccattgcgcttctccggaaacattagc

SEQ ID NO: 426 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatcccactgcgcctgatcctgaacataaaccattgcgcttctccggaaacattagc

SEQ ID NO: 427 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccatgccgactcgcgccagatcataacgtacattgcgcttctccggaaacattagc

SEQ ID NO: 428 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtaccaacagagacacccggcgtgtagctacattgcgcttctccggaaacattagc

SEQ ID NO: 429 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcaaagccgcgaatgatcaggtggtcatacattgcgcttctccggaaacattagc

SEQ ID NO: 430 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgaacttctttcagcggttcggtggtcggacattgcgcttctccggaaacattagc

SEQ ID NO: 431 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgctaaccgctgtggctactacaactgcgacattgcgcttctccggaaacattagc

SEQ ID NO: 432 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggttcaaccgctgcctgtgcataaacagacattgcgcttctccggaaacattagc

SEQ ID NO: 433 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccggtttccggctcgttctggaagtaagacattgcgcttctccggaaacattagc

SEQ ID NO: 434 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcttctgaccctttctgctgggcattggcacattgcgcttctccggaaacattagc

SEQ ID NO: 435 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagcaaagcgcaggttctgacgcacagtaacattgcgcttctccggaaacattagc

SEQ ID NO: 436 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatctcgccaccttcaaccgagaagaaggaacattgcgcttctccggaaacattagc

SEQ ID NO: 437 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagcgtcagacctgaaagcggaccgtcaaacattgcgcttctccggaaacattagc

SEQ ID NO: 438 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgaggaagctgctgcggcaaataagccacttcattgcgcttctccggaaacattagc

SEQ ID NO: 439 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgacagcacatcaacctggtgggcgatatcattgcgcttctccggaaacattagc

SEQ ID NO: 440 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgagcagagtggatttaccagaaccgttgtgcattgcgcttctccggaaacattagc

SEQ ID NO: 441 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccccatcgggatgccataaatcatttcgaggcattgcgcttctccggaaacattagc

SEQ ID NO: 442 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaaacgctttgctgctccagctttccagcgcattgcgcttctccggaaacattagc

SEQ ID NO: 443 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgcgcagggcgaccagataatcacagtagcattgcgcttctccggaaacattagc

SEQ ID NO: 444 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccacgggtaacgaccaatcgccaccagttccattgcgcttctccggaaacattagc

SEQ ID NO: 445 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgaggtcggttcgtcgagcaacagacaaccattgcgcttctccggaaacattagc

SEQ ID NO: 446 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgaccggtcactttcccggcaggaaaggttacattgcgcttctccggaaacattagc

SEQ ID NO: 447 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaatgaccgtcaggccacgctcctgacttacattgcgcttctccggaaacattagc

SEQ ID NO: 448 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtgcgcccgggcacacgaaaggagatattacattgcgcttctccggaaacattagc

SEQ ID NO: 449 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgacgttcgccgccagagagactatcgaccacattgcgcttctccggaaacattagc

SEQ ID NO: 450 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcataatttccgcaggcgttccctgagcaacattgcgcttctccggaaacattagc

SEQ ID NO: 451 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctagtgcggaacggtcaggcgttcagtatttcattgcgcttctccggaaacattagc

SEQ ID NO: 452 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggagaggccagattgctgcctatggcaatcattgcgcttctccggaaacattagc

SEQ ID NO: 453 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctaccaaacagcatgatgtcgagatccagcgcattgcgcttctccggaaacattagc

SEQ ID NO: 454 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatcaggaaacaccaactccggcgcgatttccattgcgcttctccggaaacattagc

SEQ ID NO: 455 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccacggctgcgtttaagtaatcgggttgatccattgcgcttctccggaaacattagc

SEQ ID NO: 456 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggtaagaatgtggctttcagggatatcgccattgcgcttctccggaaacattagc

SEQ ID NO: 457 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagtagctcttcaggtgcaagagaggtttcccattgcgcttctccggaaacattagc

SEQ ID NO: 458 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcggccacagcataaatccacgattcttcacattgcgcttctccggaaacattagc

SEQ ID NO: 459 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctttgcggacgcgaccttgctgcaattcaacattgcgcttctccggaaacattagc

SEQ ID NO: 460 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgctcgatcacctctgccagtgtctgacgttcattgcgcttctccggaaacattagc

SEQ ID NO: 461 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaccgatatcgacgacgcagacacccagttcattgcgcttctccggaaacattagc

SEQ ID NO: 462 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggcgtgccaaaggcgtaagcgatatcactcattgcgcttctccggaaacattagc

SEQ ID NO: 463 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctactaaagcggcaaccttcgcggtaccaatcattgcgcttctccggaaacattagc

SEQ ID NO: 464 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcacgcccgaaagtcctaccggattcttgcattgcgcttctccggaaacattagc

SEQ ID NO: 465 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgccatatcgttgtgacatgtgatcaggtgcattgcgcttctccggaaacattagc

SEQ ID NO: 466 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggcaccataccaatttcattctggcagctgcattgcgcttctccggaaacattagc

SEQ ID NO: 467 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccctttatccataccacgcgacgggcagctgcattgcgcttctccggaaacattagc

SEQ ID NO: 468 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcctgatagtcaatcgcatactcttgcgggcattgcgcttctccggaaacattagc

SEQ ID NO: 469 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccagcataaggaattaccttagtgtggcgcattgcgcttctccggaaacattagc

SEQ ID NO: 470 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcactctttcccatagtgaagcaatcccaccgcattgcgcttctccggaaacattagc

SEQ ID NO: 471 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctacggctcctgagcataatccgttaaaccgcattgcgcttctccggaaacattagc

SEQ ID NO: 472 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccaatgatattgaccataccgtcgggcagcattgcgcttctccggaaacattagc

SEQ ID NO: 473 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagttggtcaactttcagcccacaacgttccattgcgcttctccggaaacattagc

SEQ ID NO: 474 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgacaatctgccatcaattctgcctggtccattgcgcttctccggaaacattagc

SEQ ID NO: 475 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctatctcttcgttgaccaggttgagcagctccattgcgcttctccggaaacattagc

SEQ ID NO: 476 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccccagcgcacaaccgtggcgaactttaatccattgcgcttctccggaaacattagc

SEQ ID NO: 477 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaataccgaataacttgatgccagtccggccattgcgcttctccggaaacattagc

SEQ ID NO: 478 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcacttgcgtatgaaacacgcgctgagcaccattgcgcttctccggaaacattagc

SEQ ID NO: 479 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcagccaactattgagtcgcttgatccaccattgcgcttctccggaaacattagc

SEQ ID NO: 480 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccttcgatctgcgctgcgccaccggttaatacattgcgcttctccggaaacattagc

SEQ ID NO: 481 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcgttgtacgcacttgaccacggattcgacattgcgcttctccggaaacattagc

SEQ ID NO: 482 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgttctggcggcacgctgaaggtatttcctcattgcgcttctccggaaacattagc

SEQ ID NO: 483 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccttccgggccatacagcattggaagcacctcattgcgcttctccggaaacattagc

SEQ ID NO: 484 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctttcgcgatagccctgcaacacttcattggcattgcgcttctccggaaacattagc

SEQ ID NO: 485 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgttgctgagtagattcctctggcggcaaggcattgcgcttctccggaaacattagc

SEQ ID NO: 486 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccccaatgccaggatcgactgccggatatcgcattgcgcttctccggaaacattagc

SEQ ID NO: 487 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctaatccacggcaggcgttgttctatttgcgcattgcgcttctccggaaacattagc

SEQ ID NO: 488 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgttgacatcctgggtcataaaggtacccgcattgcgcttctccggaaacattagc

SEQ ID NO: 489 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatcgttattcagcgtcaactgccaggaacgcattgcgcttctccggaaacattagc

SEQ ID NO: 490 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccagacgcgttccattattgcggcgagaagcattgcgcttctccggaaacattagc

SEQ ID NO: 491 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggtcatcgccgcttccttcagagtaaatccattgcgcttctccggaaacattagc

SEQ ID NO: 492 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgtagctaatccgtttgccatcggtttgccattgcgcttctccggaaacattagc

SEQ ID NO: 493 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtctaccatatgttgatcattccaccgcgccattgcgcttctccggaaacattagc

SEQ ID NO: 494 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcaattcatcaggccactgctttctgacgccattgcgcttctccggaaacattagc

SEQ ID NO: 495 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagcctactgccgctccagagtcataacgccattgcgcttctccggaaacattagc

SEQ ID NO: 496 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgtgtgtaatggcgttcaccggtcaacaccattgcgcttctccggaaacattagc

SEQ ID NO: 497 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcttcttcgctgtttcgcgtgttcagagcacattgcgcttctccggaaacattagc

SEQ ID NO: 498 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagcaaatgcacggatggtgttacctaccgtcattgcgcttctccggaaacattagc

SEQ ID NO: 499 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgcacagcgcctttcagtacatcgttcgctcattgcgcttctccggaaacattagc

SEQ ID NO: 500 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagagaagtaccgataaccacagtcgcgttgcattgcgcttctccggaaacattagc

SEQ ID NO: 501 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgctgaacctgcttattggtcaccagagtgcattgcgcttctccggaaacattagc

SEQ ID NO: 502 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggttacgcgcagctcgtcattcatatccgcattgcgcttctccggaaacattagc

SEQ ID NO: 503 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagggagataccgcggcccagaactttcagcattgcgcttctccggaaacattagc

SEQ ID NO: 504 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcgccagacaggtcgatatcttccagcagcattgcgcttctccggaaacattagc

SEQ ID NO: 505 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaggtcgaagcccgccgtgatgttaaccagcattgcgcttctccggaaacattagc

SEQ ID NO: 506 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaggacgtttgtccatgccgatacctgtcgccattgcgcttctccggaaacattagc

SEQ ID NO: 507 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagagatagccatttcagcagcttcttccgccattgcgcttctccggaaacattagc

SEQ ID NO: 508 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccaccacccatacccgcagcaataaagacccattgcgcttctccggaaacattagc

SEQ ID NO: 509 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccttcagcgacgactggtgctgcacctgtaccattgcgcttctccggaaacattagc

SEQ ID NO: 510 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagcgcccagtcctttggtgataccgctaccattgcgcttctccggaaacattagc

SEQ ID NO: 511 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcagtttgcggcgcattgtcattcacgaccattgcgcttctccggaaacattagc

SEQ ID NO: 512 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccattacggtgcgtacgtctgcaaagtccaccattgcgcttctccggaaacattagc

SEQ ID NO: 513 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcaaacccggacgagtaatcagttcagcgacattgcgcttctccggaaacattagc

SEQ ID NO: 514 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgaaaggcttagtgacgacagcaacggtcacattgcgcttctccggaaacattagc

SEQ ID NO: 515 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgacgccgatgactttaatcaccgcgtcacattgcgcttctccggaaacattagc

SEQ ID NO: 516 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccatcccatgctgctggtagcgatccatcacattgcgcttctccggaaacattagc

SEQ ID NO: 517 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgtcgttcgggatagtgatcagagagtccacattgcgcttctccggaaacattagc

SEQ ID NO: 518 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagcgcttgtgcatcggtatttaccgcgaacattgcgcttctccggaaacattagc

SEQ ID NO: 519 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccttcaatgcgctcgcgcaccatgtgttcaacattgcgcttctccggaaacattagc

SEQ ID NO: 520 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgacccggtttggtgtctggcatattgctttcattgcgcttctccggaaacattagc

SEQ ID NO: 521 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccatcacttccggcgaagcggtttctgctttcattgcgcttctccggaaacattagc

SEQ ID NO: 522 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctggtgcccaggagtttggcgagttaaaggtcattgcgcttctccggaaacattagc

SEQ ID NO: 523 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccatgataagagacaccacggcaggttacgtcattgcgcttctccggaaacattagc

SEQ ID NO: 524 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgtgttcgtcagacgggattgcgttcggatcattgcgcttctccggaaacattagc

SEQ ID NO: 525 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccggaaccaccagttcgccattacgacgatcattgcgcttctccggaaacattagc

SEQ ID NO: 526 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaccgctaatcagcagtgcgaccaccagtgcattgcgcttctccggaaacattagc

SEQ ID NO: 527 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgccggagcgtaaataactggcactttctgcattgcgcttctccggaaacattagc

SEQ ID NO: 528 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcccactttaatcggcgtcacttcattggcattgcgcttctccggaaacattagc

SEQ ID NO: 529 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgctggctgtttacatggcagaccaaatggcattgcgcttctccggaaacattagc

SEQ ID NO: 530 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaatcgcaatcagtcgaccatcattgaccggcattgcgcttctccggaaacattagc

SEQ ID NO: 531 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttgagcacgcctttattggcgaaggtttcgcattgcgcttctccggaaacattagc

SEQ ID NO: 532 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacatatccgcaccgctcaaatctttcgtcgcattgcgcttctccggaaacattagc

SEQ ID NO: 533 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgttatcggcgttaatttgtttcagcggcgcattgcgcttctccggaaacattagc

SEQ ID NO: 534 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgggctgttagcgtgacgagtaatcgtcgcgcattgcgcttctccggaaacattagc

SEQ ID NO: 535 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctagtcgcccatcttcgtaccaaatgaaccgcattgcgcttctccggaaacattagc

SEQ ID NO: 536 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgagcatcacaaggccagcgatagggtagcattgcgcttctccggaaacattagc

SEQ ID NO: 537 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgcggatcgttaaatccggcccaggtcagcattgcgcttctccggaaacattagc

SEQ ID NO: 538 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaggcgctggtgagcggtacacaggtaaagcattgcgcttctccggaaacattagc

SEQ ID NO: 539 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgttacccgggaagaccagcgtaccctgttccattgcgcttctccggaaacattagc

SEQ ID NO: 540 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatcggtgataatcggtggcgaagtcggttccattgcgcttctccggaaacattagc

SEQ ID NO: 541 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgtgccggaagatccatgtcccacaggtccattgcgcttctccggaaacattagc

SEQ ID NO: 542 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggtcacgcccatcggcagatagaccagatccattgcgcttctccggaaacattagc

SEQ ID NO: 543 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaggatcgtaatgccatttctctttgccgccattgcgcttctccggaaacattagc

SEQ ID NO: 544 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttacttcacatcatccggcagcgcataagccattgcgcttctccggaaacattagc

SEQ ID NO: 545 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgaacggtctggtagctccacgccagtttcccattgcgcttctccggaaacattagc

SEQ ID NO: 546 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcctgcggagatcaccacatactgcttacccattgcgcttctccggaaacattagc

SEQ ID NO: 547 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgccgtagcggcgataaacagcacgttaccattgcgcttctccggaaacattagc

SEQ ID NO: 548 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccttcataggtcattggcgtagcctgaccaccattgcgcttctccggaaacattagc

SEQ ID NO: 549 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggcaatcgccacttcacgatttggatcaaccattgcgcttctccggaaacattagc

SEQ ID NO: 550 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggagtcagcgcccagaagtcgaaaccaaccattgcgcttctccggaaacattagc

SEQ ID NO: 551 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggaacgggttgagcgtgacaccatacggtacattgcgcttctccggaaacattagc

SEQ ID NO: 552 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccgttgctcatgttgtaagcgcgcaggtacattgcgcttctccggaaacattagc

SEQ ID NO: 553 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggatcgttcggctgcttcacatcgccagtacattgcgcttctccggaaacattagc

SEQ ID NO: 554 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgaccagccagcctccgccaatgagtagatacattgcgcttctccggaaacattagc

SEQ ID NO: 555 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgccttcatagcgcatctggtggaacatcacattgcgcttctccggaaacattagc

SEQ ID NO: 556 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgttgaccttcctgattacgaccataggcacattgcgcttctccggaaacattagc

SEQ ID NO: 557 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccctgcggcgtaccaatacgtttcttccacacattgcgcttctccggaaacattagc

SEQ ID NO: 558 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccaggaccacgcgggatcagtttcgaaacacattgcgcttctccggaaacattagc

SEQ ID NO: 559 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccccgcccagcatcggcatacccatattgaacattgcgcttctccggaaacattagc

SEQ ID NO: 560 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgacaaacggcaggatcagccagatgccgaacattgcgcttctccggaaacattagc

SEQ ID NO: 561 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgatcacgccagacgtttcgcgggttgagaacattgcgcttctccggaaacattagc

SEQ ID NO: 562 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccacgccggaccagctctgacctgaatgtttcattgcgcttctccggaaacattagc

SEQ ID NO: 563 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgctttcgcaacgaactgcacgaagtgttcattgcgcttctccggaaacattagc

SEQ ID NO: 564 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgtgcctacggtgtccatatttgcggcgatcattgcgcttctccggaaacattagc

SEQ ID NO: 565 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaccagtcactacgttaccagcacaaatggcattgcgcttctccggaaacattagc

SEQ ID NO: 566 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaaacgaagtttaatgccgggttcaggtcgcattgcgcttctccggaaacattagc

SEQ ID NO: 567 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaaatttctcgccgttctcctcaacgatgcgcattgcgcttctccggaaacattagc

SEQ ID NO: 568 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagagtggagcgtttagggcggatgagaacgcattgcgcttctccggaaacattagc

SEQ ID NO: 569 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctggtgcagccaccatcgctgacgatcattccattgcgcttctccggaaacattagc

SEQ ID NO: 570 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaacgatatcggcacctgagaggataagctccattgcgcttctccggaaacattagc

SEQ ID NO: 571 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccttgttcctgcacacgaataaacgtggtgccattgcgcttctccggaaacattagc

SEQ ID NO: 572 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcattgacgttccagttcaacatcggaacggccattgcgcttctccggaaacattagc

SEQ ID NO: 573 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgacgcgagttgtacaaacagaacctgggcccattgcgcttctccggaaacattagc

SEQ ID NO: 574 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccaacgtgacgtttcatcgcagactcggacattgcgcttctccggaaacattagc

SEQ ID NO: 575 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcagcatcggcacattcgattaccgcagacattgcgcttctccggaaacattagc

SEQ ID NO: 576 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgaaatccgcatcagacgtaccggtagaaacattgcgcttctccggaaacattagc

SEQ ID NO: 577 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggcagtcagcgtgaccatggtaacacccttcattgcgcttctccggaaacattagc

SEQ ID NO: 578 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaccatatccatggtcgggaccagttcggtcattgcgcttctccggaaacattagc

SEQ ID NO: 579 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcttcttctgccgcttccagcagaccttctcattgcgcttctccggaaacattagc

SEQ ID NO: 580 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccacttccatttcggttggtgcaccaaagctcattgcgcttctccggaaacattagc

SEQ ID NO: 581 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgaactctggcagcggcggaacacagttcatcattgcgcttctccggaaacattagc

SEQ ID NO: 582 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgtgttgtcagctcatccagcgtttcgtgcattgcgcttctccggaaacattagc

SEQ ID NO: 583 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctattgatatcttccatgctgtgcgccacggcattgcgcttctccggaaacattagc

SEQ ID NO: 584 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgctacgcggaaaccggtcatcacttcatcgcattgcgcttctccggaaacattagc

SEQ ID NO: 585 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttgccatcaacatcgtacagataagcgccgcattgcgcttctccggaaacattagc

SEQ ID NO: 586 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgaggcaggttaaatctggcactacgccgcattgcgcttctccggaaacattagc

SEQ ID NO: 587 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctcaaatgcggcgcgtacagaagccagatccattgcgcttctccggaaacattagc

SEQ ID NO: 588 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaacttcgcaaacacccgacgtgcagcatccattgcgcttctccggaaacattagc

SEQ ID NO: 589 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcttaaagcgttccacgtcacaggccatccattgcgcttctccggaaacattagc

SEQ ID NO: 590 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaggtatatttggcgaaatctgccggaacgccattgcgcttctccggaaacattagc

SEQ ID NO: 591 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctcatggtcgcttcagtgccggagttcacccattgcgcttctccggaaacattagc

SEQ ID NO: 592 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccagcgcatccattacatcacgacgaccaccattgcgcttctccggaaacattagc

SEQ ID NO: 593 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcttcaaacgctgacggtgccaggtaaaccattgcgcttctccggaaacattagc

SEQ ID NO: 594 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgctgcgcgacttcattcagacaggcgaaaccattgcgcttctccggaaacattagc

SEQ ID NO: 595 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccatcgcaatcgggttaccggaaagcgtacattgcgcttctccggaaacattagc

SEQ ID NO: 596 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctaccgaacatgccgccaacgtggttaacgacattgcgcttctccggaaacattagc

SEQ ID NO: 597 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctaacgtgagtgcgccagaaccggctttcacattgcgcttctccggaaacattagc

SEQ ID NO: 598 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgccaccggctcgacgataatacaggcaacattgcgcttctccggaaacattagc

SEQ ID NO: 599 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgatatacggcagatgacggtaacgctgtcattgcgcttctccggaaacattagc

SEQ ID NO: 600 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccatatcgctgccgctgtctttgtaacgctcattgcgcttctccggaaacattagc

SEQ ID NO: 601 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggttcgcgcaggcttaagatctcacgcactcattgcgcttctccggaaacattagc

SEQ ID NO: 602 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcatgagatacctgaacttcacggcacaggcattgcgcttctccggaaacattagc

SEQ ID NO: 603 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtttatccagcagcgtacaaatcgccagcgcattgcgcttctccggaaacattagc

SEQ ID NO: 604 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaaattctaccgggacgttcacttcacgacgcattgcgcttctccggaaacattagc

SEQ ID NO: 605 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatcttcaacaatcagcacgtccttgccacgcattgcgcttctccggaaacattagc

SEQ ID NO: 606 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgctaccgtagctggaggcggtcataaagcattgcgcttctccggaaacattagc

SEQ ID NO: 607 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcaatgccgtaacccaccacaaactcatcccattgcgcttctccggaaacattagc

SEQ ID NO: 608 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaaacataaatgagccacgcagcagacccaccattgcgcttctccggaaacattagc

SEQ ID NO: 609 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgacgacccagttcggcgatacgcgctttaacattgcgcttctccggaaacattagc

SEQ ID NO: 610 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggtattcccggctgttagcgcttcagatttcattgcgcttctccggaaacattagc

SEQ ID NO: 611 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgttcttgcaaacgcgagttaaagcgactgcattgcgcttctccggaaacattagc

SEQ ID NO: 612 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccaataatacgcagacgcacgttatgtcgcattgcgcttctccggaaacattagc

SEQ ID NO: 613 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaaggcatacagcgttaacgcctcaataccgcattgcgcttctccggaaacattagc

SEQ ID NO: 614 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccaccgtagttcgccgcaatattcagcgtccattgcgcttctccggaaacattagc

SEQ ID NO: 615 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatctgatctggttgcaggtttccttgctgccattgcgcttctccggaaacattagc

SEQ ID NO: 616 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggctttatgcccaaaggcacgaatcttccccattgcgcttctccggaaacattagc

SEQ ID NO: 617 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccagttgcctgactccctggactatatccccattgcgcttctccggaaacattagc

SEQ ID NO: 618 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtcttgttcatcgaaatcgggccagagaaccattgcgcttctccggaaacattagc

SEQ ID NO: 619 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgctatcgagcgcccacacaaacagttccacattgcgcttctccggaaacattagc

SEQ ID NO: 620 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccgccgaaacgacgctctcgattagcaaacattgcgcttctccggaaacattagc

SEQ ID NO: 621 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccatgcgtagcgccaggtacaggctttcttcattgcgcttctccggaaacattagc

SEQ ID NO: 622 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgaggcaggcagaaaccgcgtgttgttcattcattgcgcttctccggaaacattagc

SEQ ID NO: 623 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgaggtatcgccgtggaaaccatctttgatcattgcgcttctccggaaacattagc

SEQ ID NO: 624 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttattcgtcgtgcgagattatcgccgggatcattgcgcttctccggaaacattagc

SEQ ID NO: 625 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgacgcccggtttaacatacggttcgatcatcattgcgcttctccggaaacattagc

SEQ ID NO: 626 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagagatgcaaacggatttcggatagccgtgcattgcgcttctccggaaacattagc

SEQ ID NO: 627 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctaatcattacagatgcgatccagctcgccgcattgcgcttctccggaaacattagc

SEQ ID NO: 628 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatcgccatctttcagcagcttagcatcgtccattgcgcttctccggaaacattagc

SEQ ID NO: 629 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgttgaccattggctcgatggtgaacgtccattgcgcttctccggaaacattagc

SEQ ID NO: 630 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccgtccagccatctttcatggtgcggatccattgcgcttctccggaaacattagc

SEQ ID NO: 631 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccatccttgcgtagcgtcagaatttcgcagccattgcgcttctccggaaacattagc

SEQ ID NO: 632 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctccgcaatattcacgaacgacggagaagcccattgcgcttctccggaaacattagc

SEQ ID NO: 633 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgcggttcttcatggaagccgcgaccaatacattgcgcttctccggaaacattagc

SEQ ID NO: 634 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaggtttcagtacgacgttggtttcacgggacattgcgcttctccggaaacattagc

SEQ ID NO: 635 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccttcgacaaatttctgaatcgccgcaccgacattgcgcttctccggaaacattagc

SEQ ID NO: 636 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcccatgatggtcggcttaccgacgataacattgcgcttctccggaaacattagc

SEQ ID NO: 637 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcctgcgggaagctctgatacagcactttcattgcgcttctccggaaacattagc

SEQ ID NO: 638 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcttacgccctttgcctttgcccttaccttcattgcgcttctccggaaacattagc

SEQ ID NO: 639 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcaattggcgcatccgcaatccacgtattcattgcgcttctccggaaacattagc

SEQ ID NO: 640 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagcggacgggcactcaacatgtcatagagtcattgcgcttctccggaaacattagc

SEQ ID NO: 641 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggcttagctcttctaccgggcgaccaaagtcattgcgcttctccggaaacattagc

SEQ ID NO: 642 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatgatcagcgccatgtaagcttcgttcgctcattgcgcttctccggaaacattagc

SEQ ID NO: 643 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccaccagcatcactctgccgctttcgctatcattgcgcttctccggaaacattagc

SEQ ID NO: 644 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggatttcgttgtcgccgctctgaccgagatcattgcgcttctccggaaacattagc

SEQ ID NO: 645 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctggcaccatacagtttggtcggctggttgcattgcgcttctccggaaacattagc

SEQ ID NO: 646 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctttcaccataccgactaacagcgcctgctgcattgcgcttctccggaaacattagc

SEQ ID NO: 647 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacggacggcgaatcatctcaatgctgttggcattgcgcttctccggaaacattagc

SEQ ID NO: 648 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggtcatcattctgcggtgaccagacctggcattgcgcttctccggaaacattagc

SEQ ID NO: 649 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagatttaccgtcggcacgttcatcgaacggcattgcgcttctccggaaacattagc

SEQ ID NO: 650 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggtcatgtctggctcaagattgaccattcgcattgcgcttctccggaaacattagc

SEQ ID NO: 651 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaaactgcggctcagaacctccgaccatcgcattgcgcttctccggaaacattagc

SEQ ID NO: 652 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgctcaagaatacggtctttgctgtaacgcgcattgcgcttctccggaaacattagc

SEQ ID NO: 653 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaacatatccttgatccaaccggctacaccgcattgcgcttctccggaaacattagc

SEQ ID NO: 654 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccagtaagaacgctcgctggagaggaacagcattgcgcttctccggaaacattagc

SEQ ID NO: 655 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatggtgatcagacgcggatcaagacggaagcattgcgcttctccggaaacattagc

SEQ ID NO: 656 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaaacagacgctgctcaccgtttggcgaagcattgcgcttctccggaaacattagc

SEQ ID NO: 657 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtcgcctccagcagcttcaccatctcgttccattgcgcttctccggaaacattagc

SEQ ID NO: 658 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgtcggtttccctttgcgtccaattggctccattgcgcttctccggaaacattagc

SEQ ID NO: 659 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggctgttgctgcggctgagaagactgatccattgcgcttctccggaaacattagc

SEQ ID NO: 660 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacggttgttctccatattgacgatcgtcgccattgcgcttctccggaaacattagc

SEQ ID NO: 661 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggctggcttaaggccgtcagataagtcgccattgcgcttctccggaaacattagc

SEQ ID NO: 662 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcgagataaacgccgtaaatggcgatcagccattgcgcttctccggaaacattagc

SEQ ID NO: 663 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcataatctgttgctgttgcagcagacgcagccattgcgcttctccggaaacattagc

SEQ ID NO: 664 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgacggtcttctgtcgccagcaaagtatcccattgcgcttctccggaaacattagc

SEQ ID NO: 665 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcgctcaacttacgctgtttcttcagtgcccattgcgcttctccggaaacattagc

SEQ ID NO: 666 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgttgcgtcagcgtactcgcaccctgtaccattgcgcttctccggaaacattagc

SEQ ID NO: 667 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacaagattcagcggcgttggcgtctggttacattgcgcttctccggaaacattagc

SEQ ID NO: 668 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtttgccagcaccgcacgtccgattgagtacattgcgcttctccggaaacattagc

SEQ ID NO: 669 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgccgtcaatgcccgcaaaccaggtatctacattgcgcttctccggaaacattagc

SEQ ID NO: 670 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcgttgtaccacctgctgcatggtccatacattgcgcttctccggaaacattagc

SEQ ID NO: 671 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcaggcagttgccagaccttgccatcaatacattgcgcttctccggaaacattagc

SEQ ID NO: 672 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgtcgtagtccacgcccatatctgcaatacattgcgcttctccggaaacattagc

SEQ ID NO: 673 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacttcaccactaaagcggtcgacgaccacacattgcgcttctccggaaacattagc

SEQ ID NO: 674 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtacgcccagtttaatccaggtctccgtaacattgcgcttctccggaaacattagc

SEQ ID NO: 675 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccctgcagctcctgacgcaccagttgcataacattgcgcttctccggaaacattagc

SEQ ID NO: 676 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtcctgggccaccgagtcaaaggtagtgaacattgcgcttctccggaaacattagc

SEQ ID NO: 677 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgcacctgtccttctttactgtccgggaacattgcgcttctccggaaacattagc

SEQ ID NO: 678 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcaagatacgcatgccaccgctgcaaacaacattgcgcttctccggaaacattagc

SEQ ID NO: 679 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttcgtccgagacaacgatatcgcccactttcattgcgcttctccggaaacattagc

SEQ ID NO: 680 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgccggacagcctggtaactgaccgtattcattgcgcttctccggaaacattagc

SEQ ID NO: 681 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaggcctcagcggcagcgatcagtttatcgcattgcgcttctccggaaacattagc

SEQ ID NO: 682 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttcgcaaccgccgagactgatagtttgacgcattgcgcttctccggaaacattagc

SEQ ID NO: 683 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccatcaggctggactgtttagcggcaacagccattgcgcttctccggaaacattagc

SEQ ID NO: 684 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatcacatctggcttgcagtgttccaacagccattgcgcttctccggaaacattagc

SEQ ID NO: 685 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgccagaccaacagaaccgttgatgaaagccattgcgcttctccggaaacattagc

SEQ ID NO: 686 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcgaagctaagatgagactgttgatcggcccattgcgcttctccggaaacattagc

SEQ ID NO: 687 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctagccatgtgcaagtttctgcaccagtgaccattgcgcttctccggaaacattagc

SEQ ID NO: 688 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggagatggcgcgtacgacaacaaacgggaccattgcgcttctccggaaacattagc

SEQ ID NO: 689 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcaacctcggttccattcagttggccggtacattgcgcttctccggaaacattagc

SEQ ID NO: 690 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccatgggcgattgccgtcgcttccatctctacattgcgcttctccggaaacattagc

SEQ ID NO: 691 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgcagcgactttaccgatgcccgatttcacattgcgcttctccggaaacattagc

SEQ ID NO: 692 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgctaacaatcaggccacgtacagcgttaacattgcgcttctccggaaacattagc

SEQ ID NO: 693 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccaaatgccgtgacatccgcgtcgtgataacattgcgcttctccggaaacattagc

SEQ ID NO: 694 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccctccggagtttgcggcttcagtttgatttcattgcgcttctccggaaacattagc

SEQ ID NO: 695 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggaatacgcgcttcatgagcggcgacaattcattgcgcttctccggaaacattagc

SEQ ID NO: 696 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctttaatggtggcgtccacttccgtcgggtcattgcgcttctccggaaacattagc

SEQ ID NO: 697 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctcgcggtaacaattcgcggatcaccggatcattgcgcttctccggaaacattagc

SEQ ID NO: 698 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcggcatcttcgctgaagccgtaagtcgtgcattgcgcttctccggaaacattagc

SEQ ID NO: 699 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggtgacaatcgccaccatcggttgcagatgcattgcgcttctccggaaacattagc

SEQ ID NO: 700 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttctcaaagtcgccctggtaggtatccatgcattgcgcttctccggaaacattagc

SEQ ID NO: 701 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcttctgcgtagatgctggaaaccatcgcgcattgcgcttctccggaaacattagc

SEQ ID NO: 702 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctagcctcgtcgtcaatgccctcttccgtagcattgcgcttctccggaaacattagc

SEQ ID NO: 703 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttcattaactgctgcgtgaccggatttggccattgcgcttctccggaaacattagc

SEQ ID NO: 704 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgataaccttcattggccagaacttcggccattgcgcttctccggaaacattagc

SEQ ID NO: 705 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccattcactggctccagcgggaattcacccattgcgcttctccggaaacattagc

SEQ ID NO: 706 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgggcgatggttgaaataaatcgtcgcaccattgcgcttctccggaaacattagc

SEQ ID NO: 707 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgggccaatctgctgataatcttctacaccattgcgcttctccggaaacattagc

SEQ ID NO: 708 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccacgtccacgaattgtgcgacacagcgaaccattgcgcttctccggaaacattagc

SEQ ID NO: 709 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgttatcggcagaaatcgcgctggaaacaaccattgcgcttctccggaaacattagc

SEQ ID NO: 710 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggttaataccggtgccagcatctcggctacattgcgcttctccggaaacattagc

SEQ ID NO: 711 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcattaactcagccagcatttcggcacgacattgcgcttctccggaaacattagc

SEQ ID NO: 712 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccttcgccagccggatacacttccagcatcacattgcgcttctccggaaacattagc

SEQ ID NO: 713 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgatgcatcactctcatctgcttcggcaacattgcgcttctccggaaacattagc

SEQ ID NO: 714 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtatcaacctgcgtcagcacattggcgaaacattgcgcttctccggaaacattagc

SEQ ID NO: 715 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccatcacgaggatctgcccgtcagtgacgttcattgcgcttctccggaaacattagc

SEQ ID NO: 716 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaatgccaataaccgggatcgccagtgcttcattgcgcttctccggaaacattagc

SEQ ID NO: 717 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgaccatgttagcaccggcacgcataaccgtcattgcgcttctccggaaacattagc

SEQ ID NO: 718 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctggcgaagctatagtcataagcggtgatggcattgcgcttctccggaaacattagc

SEQ ID NO: 719 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctaggaatgtgaccgccggtaataccaaaggcattgcgcttctccggaaacattagc

SEQ ID NO: 720 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgtacttctgcagtaaggagatggtggtcgcattgcgcttctccggaaacattagc

SEQ ID NO: 721 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctggaaactgtgttcttcgcccggataaacgcattgcgcttctccggaaacattagc

SEQ ID NO: 722 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagcagcttctaaggctaatgcatcgctgagcattgcgcttctccggaaacattagc

SEQ ID NO: 723 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctccacttcagccatatactgccgcacagccattgcgcttctccggaaacattagc

SEQ ID NO: 724 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaacctaagtgaccacatacaggaacggcaccattgcgcttctccggaaacattagc

SEQ ID NO: 725 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtcagcatttgtacggtttctaccagccaccattgcgcttctccggaaacattagc

SEQ ID NO: 726 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcagtgtggtaggcgatatcggcaacggtacattgcgcttctccggaaacattagc

SEQ ID NO: 727 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccatacgccataaacggcaggtcagccagcacattgcgcttctccggaaacattagc

SEQ ID NO: 728 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccagcgaatcgcccaccagcatgacgttaacattgcgcttctccggaaacattagc

SEQ ID NO: 729 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccaggcggctaccagaattactgcccgtttcattgcgcttctccggaaacattagc

SEQ ID NO: 730 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgatgtccggctggaccaggttgaacagcttcattgcgcttctccggaaacattagc

SEQ ID NO: 731 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccttgccttccatacgcaggcggcgaatttgcattgcgcttctccggaaacattagc

SEQ ID NO: 732 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgtttgtttagcttctcgcagtcctcctgcattgcgcttctccggaaacattagc

SEQ ID NO: 733 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgtaagtgtgggtttcagtaccgttcgggcattgcgcttctccggaaacattagc

SEQ ID NO: 734 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgttgttccgccgtcagataaccgttacgcattgcgcttctccggaaacattagc

SEQ ID NO: 735 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatcgtgcaggttacccatggtaggcaccagcattgcgcttctccggaaacattagc

SEQ ID NO: 736 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagaccgtctttggcgcgcataattggcaccattgcgcttctccggaaacattagc

SEQ ID NO: 737 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgggctttggcttcgtcgaccagcttcatacattgcgcttctccggaaacattagc

SEQ ID NO: 738 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcagcagcggcagggtttcgataattaacacattgcgcttctccggaaacattagc

SEQ ID NO: 739 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaccttccagcatggtcgaaaggccaggaacattgcgcttctccggaaacattagc

SEQ ID NO: 740 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgcatgagtcactttcacgcggtggagttcattgcgcttctccggaaacattagc

SEQ ID NO: 741 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccaggtgcgagcttcttcatctggcatggtcattgcgcttctccggaaacattagc

SEQ ID NO: 742 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccgcgatggcataagtggagaaacgcttgcattgcgcttctccggaaacattagc

SEQ ID NO: 743 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcctggtcaatggcgcaagaaccttcatagcattgcgcttctccggaaacattagc

SEQ ID NO: 744 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcaagcaacctgtaccggaatcgctttcgccattgcgcttctccggaaacattagc

SEQ ID NO: 745 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagctggcgatgatgacaatatcgccgacaccattgcgcttctccggaaacattagc

SEQ ID NO: 746 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagtgggccgccgcaccgttaacagaaatacattgcgcttctccggaaacattagc

SEQ ID NO: 747 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgaaagcgtcgtgataggtcaggccgctttcattgcgcttctccggaaacattagc

SEQ ID NO: 748 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggtttccgggctgatgcgcatacccaatttcattgcgcttctccggaaacattagc

SEQ ID NO: 749 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgcaactgccagatatcgcgggttaatgtcattgcgcttctccggaaacattagc

SEQ ID NO: 750 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggaacgccgggttcacgcgcatatcgttatcattgcgcttctccggaaacattagc

SEQ ID NO: 751 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcggtgacggttcttcatccagctcgttgcattgcgcttctccggaaacattagc

SEQ ID NO: 752 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccgtctccagcagtgggtaccagaacatgcattgcgcttctccggaaacattagc

SEQ ID NO: 753 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatacaggctgttgatagtgaaatcgcggcgcattgcgcttctccggaaacattagc

SEQ ID NO: 754 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtttaagcgattcttcaaacaggcgtgccgcattgcgcttctccggaaacattagc

SEQ ID NO: 755 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacagcttataggtttcgtaaccgtagcccgcattgcgcttctccggaaacattagc

SEQ ID NO: 756 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcagttacggaacagtttgcgcacctgctccattgcgcttctccggaaacattagc

SEQ ID NO: 757 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctttccgggttaccaatcagacggataacgccattgcgcttctccggaaacattagc

SEQ ID NO: 758 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcataagccgcacggaacttaggatgctcccattgcgcttctccggaaacattagc

SEQ ID NO: 759 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcagatccttcatgccgccaacgtaatcaccattgcgcttctccggaaacattagc

SEQ ID NO: 760 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggtcgctgacgttaccttcgtggtgtccaccattgcgcttctccggaaacattagc

SEQ ID NO: 761 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtcgcaacttcgataatctccgggccaaaccattgcgcttctccggaaacattagc

SEQ ID NO: 762 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccaaccagccaggcttcgtatcccgctttacattgcgcttctccggaaacattagc

SEQ ID NO: 763 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgcagcatacgtaccggatcttcacggtacattgcgcttctccggaaacattagc

SEQ ID NO: 764 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccatgcgcgtttaccctgacgacgggacatacattgcgcttctccggaaacattagc

SEQ ID NO: 765 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcgtatcggtattcttcagcacctgttcacattgcgcttctccggaaacattagc

SEQ ID NO: 766 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgttacgctcaacttcagctcgcaaggccacattgcgcttctccggaaacattagc

SEQ ID NO: 767 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgtgaagtagcgggtaatggtcgggaacacattgcgcttctccggaaacattagc

SEQ ID NO: 768 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccggatcagcggtaaacacgccgtcaacttcattgcgcttctccggaaacattagc

SEQ ID NO: 769 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcaacaggctgatagcttctgcccagctgtcattgcgcttctccggaaacattagc

SEQ ID NO: 770 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccagcacttcgctgtaagtcagttgctcgtcattgcgcttctccggaaacattagc

SEQ ID NO: 771 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgctttcagcaccacatcggcttcaatttcgcattgcgcttctccggaaacattagc

SEQ ID NO: 772 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccaatcaccacaccaacctgaatacccagcattgcgcttctccggaaacattagc

SEQ ID NO: 773 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttcctgagccatacgatccagtatgcttgccattgcgcttctccggaaacattagc

SEQ ID NO: 774 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgaatcggtaatttatggtcacgagccagccattgcgcttctccggaaacattagc

SEQ ID NO: 775 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcgcacacgccattcaatggaatagcggaccattgcgcttctccggaaacattagc

SEQ ID NO: 776 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtgccctgcagagcttcgccactcaacttacattgcgcttctccggaaacattagc

SEQ ID NO: 777 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcaggcaagctgctgagtcggtggtaaagacattgcgcttctccggaaacattagc

SEQ ID NO: 778 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttacctgtaccggcggagaggatcaccacacattgcgcttctccggaaacattagc

SEQ ID NO: 779 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccatcgactttaacgatcctgtcgcctgctcattgcgcttctccggaaacattagc

SEQ ID NO: 780 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccttcgacgatggcgttgaacggcccatactcattgcgcttctccggaaacattagc

SEQ ID NO: 781 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttggcgatagagatcggcccactgaggttgcattgcgcttctccggaaacattagc

SEQ ID NO: 782 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgtaaatcgagctttacatcccgccgttgcattgcgcttctccggaaacattagc

SEQ ID NO: 783 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgggcaacccagaaatgaccaaattcatgcattgcgcttctccggaaacattagc

SEQ ID NO: 784 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcatcacaaaggtcacccactgcgttaaggcattgcgcttctccggaaacattagc

SEQ ID NO: 785 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctattattgaaggcatggtggcggagttccgcattgcgcttctccggaaacattagc

SEQ ID NO: 786 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcgctttaccattacccggtttactctccgcattgcgcttctccggaaacattagc

SEQ ID NO: 787 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctaacgctaaggatttacccgggttatcccgcattgcgcttctccggaaacattagc

SEQ ID NO: 788 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccagtacaggttcaatttgcggcccacgagcattgcgcttctccggaaacattagc

SEQ ID NO: 789 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgttcacgctaataagcgcaagaaacggcagcattgcgcttctccggaaacattagc

SEQ ID NO: 790 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcccaacgggatcagggcgataacatattccattgcgcttctccggaaacattagc

SEQ ID NO: 791 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctcatctggcaaaggaatgactttcggctccattgcgcttctccggaaacattagc

SEQ ID NO: 792 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgaccaactgcaaacgcacggcatcccaatccattgcgcttctccggaaacattagc

SEQ ID NO: 793 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgccaaatggcgctactgtaatggtggtgccattgcgcttctccggaaacattagc

SEQ ID NO: 794 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttaacagcaccagcagaatcgagccaatgccattgcgcttctccggaaacattagc

SEQ ID NO: 795 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccccagcatactgaccgtcagcttcatcagccattgcgcttctccggaaacattagc

SEQ ID NO: 796 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaagtacacccagtgcaacgatgaacgaagccattgcgcttctccggaaacattagc

SEQ ID NO: 797 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagttccgtacctggtgcaatttgtgcttcccattgcgcttctccggaaacattagc

SEQ ID NO: 798 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccggatcttctttatcaggctcaaacgcccattgcgcttctccggaaacattagc

SEQ ID NO: 799 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatcgaattggctgctatttcgccaaccacccattgcgcttctccggaaacattagc

SEQ ID NO: 800 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcttatcagttcgccgccagagcgccttaccattgcgcttctccggaaacattagc

SEQ ID NO: 801 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccctattgagaaacgctcaacgcgaacaccacattgcgcttctccggaaacattagc

SEQ ID NO: 802 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgaagtttgcaaccggacctgcggcaataacattgcgcttctccggaaacattagc

SEQ ID NO: 803 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgccgttacgtggtgcatcagcatgatgttcattgcgcttctccggaaacattagc

SEQ ID NO: 804 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgttctggatctgcacaccttctttcgcttcattgcgcttctccggaaacattagc

SEQ ID NO: 805 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccatctccggttccatggcattgatgatgtcattgcgcttctccggaaacattagc

SEQ ID NO: 806 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgttggcaatcgctttagtcacccacgggtcattgcgcttctccggaaacattagc

SEQ ID NO: 807 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctacgcatcagcgcatcttccatcgacaggtcattgcgcttctccggaaacattagc

SEQ ID NO: 808 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaggtgctctttccacagggagtcaagcgtcattgcgcttctccggaaacattagc

SEQ ID NO: 809 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgctcacgcagcgtctcttcatgcagttctcattgcgcttctccggaaacattagc

SEQ ID NO: 810 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgacgaccagaacgaccgcgcaactggttatcattgcgcttctccggaaacattagc

SEQ ID NO: 811 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgcctgccagctaccaccgagcacaatatcattgcgcttctccggaaacattagc

SEQ ID NO: 812 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgggattcgtgacgctcggtaccgatgatcattgcgcttctccggaaacattagc

SEQ ID NO: 813 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccacgggcaaggatcgttacgtcctactttgcattgcgcttctccggaaacattagc

SEQ ID NO: 814 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctatcccacatttcttccagcgactgtggtgcattgcgcttctccggaaacattagc

SEQ ID NO: 815 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggagtccacttcgtccaccagcgcatagtgcattgcgcttctccggaaacattagc

SEQ ID NO: 816 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccataccgccgagtaactgaacgtcgaagtgcattgcgcttctccggaaacattagc

SEQ ID NO: 817 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgatggagatagtacccaccagcaccggctgcattgcgcttctccggaaacattagc

SEQ ID NO: 818 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttcaacgactccagcatcgctgcaaacatgcattgcgcttctccggaaacattagc

SEQ ID NO: 819 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttatacatttccgagctgtcttctgccgggcattgcgcttctccggaaacattagc

SEQ ID NO: 820 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgcacgcctttaccggttagtgcgttcaggcattgcgcttctccggaaacattagc

SEQ ID NO: 821 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgttgttccagctcctcaacctcttcaggcattgcgcttctccggaaacattagc

SEQ ID NO: 822 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttgtcgcgcaggtagtcaaagccgtattcgcattgcgcttctccggaaacattagc

SEQ ID NO: 823 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacagacggaagtagttctggaaggtgatcgcattgcgcttctccggaaacattagc

SEQ ID NO: 824 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttatccagccactcggcaattggcaaatcgcattgcgcttctccggaaacattagc

SEQ ID NO: 825 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctacgctgtacacgttcttcagggctgaacgcattgcgcttctccggaaacattagc

SEQ ID NO: 826 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcttcgttggcgtggaatttggcgttcagcattgcgcttctccggaaacattagc

SEQ ID NO: 827 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccatgcccggcaggttgataccgacagtcagcattgcgcttctccggaaacattagc

SEQ ID NO: 828 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcttcagtcatgtagaccaggtccggcagcattgcgcttctccggaaacattagc

SEQ ID NO: 829 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttcatccatgatgccctctttcaccagcagcattgcgcttctccggaaacattagc

SEQ ID NO: 830 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaacgatgtagtcgacgtcacgggtaaacagcattgcgcttctccggaaacattagc

SEQ ID NO: 831 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgagaagtggccttcgccctggaaggtttccattgcgcttctccggaaacattagc

SEQ ID NO: 832 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccacgacccgccatattggtcgcgatagtccattgcgcttctccggaaacattagc

SEQ ID NO: 833 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaggtggataccctgacgcagatagtccatccattgcgcttctccggaaacattagc

SEQ ID NO: 834 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcagttgcttacgaatgtcgaagttacggccattgcgcttctccggaaacattagc

SEQ ID NO: 835 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctttacgctgatacacttcgatggactgcgccattgcgcttctccggaaacattagc

SEQ ID NO: 836 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacagttcgttacgctgggagtaaatggcgccattgcgcttctccggaaacattagc

SEQ ID NO: 837 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcttcgcctggcttcatacccagtttacgccattgcgcttctccggaaacattagc

SEQ ID NO: 838 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgtaccgtaagtgatgtcagctgcgtaagccattgcgcttctccggaaacattagc

SEQ ID NO: 839 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccctgcttccagtaccgcatcgtgacgtacccattgcgcttctccggaaacattagc

SEQ ID NO: 840 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcataccggctttggtcagttcgtttgacacccattgcgcttctccggaaacattagc

SEQ ID NO: 841 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgctgttaatggtttcgctcacatcgctgaccattgcgcttctccggaaacattagc

SEQ ID NO: 842 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgaatcattggacggttggtcggaacaaccattgcgcttctccggaaacattagc

SEQ ID NO: 843 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtacgcatttcggcgatgcagcgttcgttacattgcgcttctccggaaacattagc

SEQ ID NO: 844 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctaccaaagacgcgcttacttgcctcacgtacattgcgcttctccggaaacattagc

SEQ ID NO: 845 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgcatttgcgctaaacgctcggcttccatacattgcgcttctccggaaacattagc

SEQ ID NO: 846 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcatgacgcctttctcgaagtgacgcatcacattgcgcttctccggaaacattagc

SEQ ID NO: 847 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccacgttgcgccaggtagtcgttgacggtaacattgcgcttctccggaaacattagc

SEQ ID NO: 848 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggctgcagagtcgtcatcctgatggctaacattgcgcttctccggaaacattagc

SEQ ID NO: 849 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccctgcatggtacgaccggtgtgttcgtcaacattgcgcttctccggaaacattagc

SEQ ID NO: 850 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccatccctaataagagatgcggccagaagcattgcgcttctccggaaacattagc

SEQ ID NO: 851 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagcgcgctgagcgtatccagtaatgcaagcattgcgcttctccggaaacattagc

SEQ ID NO: 852 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttcgcttccagcaaggccaattgaccaaagcattgcgcttctccggaaacattagc

SEQ ID NO: 853 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgttgctgagcgcaggcaaacctaaactccattgcgcttctccggaaacattagc

SEQ ID NO: 854 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaatcaacggaatagttcgaattcgggcggccattgcgcttctccggaaacattagc

SEQ ID NO: 855 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgcctgaagaggcaaagattcttcagcaacattgcgcttctccggaaacattagc

SEQ ID NO: 856 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccagaatagtgacgctggcaccctgtggttcattgcgcttctccggaaacattagc

SEQ ID NO: 857 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcggagaaatcacgccgcaggtagagacttcattgcgcttctccggaaacattagc

SEQ ID NO: 858 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgctgtcggttaagtcttccggtttggtgtcattgcgcttctccggaaacattagc

SEQ ID NO: 859 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttctttccacagcgcagcggtaatttcctgcattgcgcttctccggaaacattagc

SEQ ID NO: 860 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgctggcatattgcgcccgtaataaatctcgcattgcgcttctccggaaacattagc

SEQ ID NO: 861 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagacgttcacatccaccatgtcatacagcgcattgcgcttctccggaaacattagc

SEQ ID NO: 862 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacaggtttctgacaggatttcggtcagacgcattgcgcttctccggaaacattagc

SEQ ID NO: 863 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggcgcggaaggtacataaaccgccttcagcattgcgcttctccggaaacattagc

SEQ ID NO: 864 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatcaagatgggcaacgaccgtttctggcagcattgcgcttctccggaaacattagc

SEQ ID NO: 865 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtcgatgagtttcgggtcaaccggttcttccattgcgcttctccggaaacattagc

SEQ ID NO: 866 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcataagcaatataaccgtcgcgctcttcggccattgcgcttctccggaaacattagc

SEQ ID NO: 867 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctcaagctggtggatcaggtaattcagcgccattgcgcttctccggaaacattagc

SEQ ID NO: 868 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgcgcacgcgataatcaatggttacgatacattgcgcttctccggaaacattagc

SEQ ID NO: 869 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccaacgtcgctgaagtaatggctgagtttcattgcgcttctccggaaacattagc

SEQ ID NO: 870 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggatcgtcgtagctaccggcgttatggttcattgcgcttctccggaaacattagc

SEQ ID NO: 871 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctagataaactcgtcgcgctcggtggtttgtcattgcgcttctccggaaacattagc

SEQ ID NO: 872 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgggccagtagcggaacatgggtcatcatctcattgcgcttctccggaaacattagc

SEQ ID NO: 873 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccgccgataatcagcacatgtttcgcgtgcattgcgcttctccggaaacattagc

SEQ ID NO: 874 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgataggatcggtgcagtcggagataatgcattgcgcttctccggaaacattagc

SEQ ID NO: 875 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgcgcctgaataatttcggttgagagatggcattgcgcttctccggaaacattagc

SEQ ID NO: 876 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgccgtatggattgccggattgtaataacggcattgcgcttctccggaaacattagc

SEQ ID NO: 877 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgatactgacggcagaacgatacgacacccgcattgcgcttctccggaaacattagc

SEQ ID NO: 878 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccatccagcgccattacgcgaccaaatgcagcattgcgcttctccggaaacattagc

SEQ ID NO: 879 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaacgaaattgacgccatcgtcgatcaccagcattgcgcttctccggaaacattagc

SEQ ID NO: 880 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatctaccgcaaagtactgcccaaactggtccattgcgcttctccggaaacattagc

SEQ ID NO: 881 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtcatgataccgccgtaataggtcgggatccattgcgcttctccggaaacattagc

SEQ ID NO: 882 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtgcgacgaagataccgccaggattcaggccattgcgcttctccggaaacattagc

SEQ ID NO: 883 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaagccagtgcgtcttgcagatactgaggtacattgcgcttctccggaaacattagc

SEQ ID NO: 884 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccaatgccgctgagatcgcggtagctgtcttcattgcgcttctccggaaacattagc

SEQ ID NO: 885 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgccgttaaatcggccatatcttcggcttcattgcgcttctccggaaacattagc

SEQ ID NO: 886 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccagacggccatcaggctgccgaataacactcattgcgcttctccggaaacattagc

SEQ ID NO: 887 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttgacgtaggcaagcaggcttaaggaatcgcattgcgcttctccggaaacattagc

SEQ ID NO: 888 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgtcgaaggtgtcgtaattactgaggtccccattgcgcttctccggaaacattagc

SEQ ID NO: 889 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccagtgcgcgacgaatttgcccgttaggtacattgcgcttctccggaaacattagc

SEQ ID NO: 890 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgactggcgaattaaatccagcaccatggcattgcgcttctccggaaacattagc

SEQ ID NO: 891 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcactgacattacggaagaaatgcagcgcgcattgcgcttctccggaaacattagc

SEQ ID NO: 892 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttagcaacaggccagttcgaaatccagacgcattgcgcttctccggaaacattagc

SEQ ID NO: 893 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttgcggataaagctgtgaatcgagcgacagcattgcgcttctccggaaacattagc

SEQ ID NO: 894 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacgccaatccaggtctggagtttacgttgccattgcgcttctccggaaacattagc

SEQ ID NO: 895 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttaatgcttcaatacggctctggtccacgccattgcgcttctccggaaacattagc

SEQ ID NO: 896 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgtaggtaaatcaaagctgcaacagccgcccattgcgcttctccggaaacattagc

SEQ ID NO: 897 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgtcaagttctttcaacagctcagtgcggaccattgcgcttctccggaaacattagc

SEQ ID NO: 898 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgaaattgcccgatacgcggcgcggaaattacattgcgcttctccggaaacattagc

SEQ ID NO: 899 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgggttcaggctggcaatccaggtttctacattgcgcttctccggaaacattagc

SEQ ID NO: 900 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgctcagtcgctgacgcaccagagcaatcaacattgcgcttctccggaaacattagc

SEQ ID NO: 901 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccccgcaggttgggcaattcaccgtaatagtcattgcgcttctccggaaacattagc

SEQ ID NO: 902 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcactgctttggttcttcgctccagtcatccattgcgcttctccggaaacattagc

SEQ ID NO: 903 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccttcagcagcccattctccgaggtcgatcacattgcgcttctccggaaacattagc

SEQ ID NO: 904 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccctcaacgccgcatagcgacaggctttcttcattgcgcttctccggaaacattagc

SEQ ID NO: 905 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccatcacctcttcaccgtccatccacagggcattgcgcttctccggaaacattagc

SEQ ID NO: 906 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgctgcttgttccacttcatcaagcaaggcattgcgcttctccggaaacattagc

SEQ ID NO: 907 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgttgccaggaccgttcgctacctttcagcattgcgcttctccggaaacattagc

SEQ ID NO: 908 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttcatcgccagcgaattccagttgattggccattgcgcttctccggaaacattagc

SEQ ID NO: 909 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctacttctgctgcacgaaattgcggtaagcccattgcgcttctccggaaacattagc

SEQ ID NO: 910 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttcacctcttcattaaaccagtgcccgacccattgcgcttctccggaaacattagc

SEQ ID NO: 911 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatggacatacgcacctttaccactccggtacattgcgcttctccggaaacattagc

SEQ ID NO: 912 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcagtcgatactgatagccatcgagcttcggtcattgcgcttctccggaaacattagc

SEQ ID NO: 913 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcagcacttcaacttccagcgtcgcggtatcattgcgcttctccggaaacattagc

SEQ ID NO: 914 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcatttctgcttcttccaggtagtgtgtggcattgcgcttctccggaaacattagc

SEQ ID NO: 915 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaccagtgaaacaaacagctcttccagacggcattgcgcttctccggaaacattagc

SEQ ID NO: 916 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgcctgattcaccacaatttgctgcacggcattgcgcttctccggaaacattagc

SEQ ID NO: 917 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcatcccgccagataacatacgcgcacgttcgcattgcgcttctccggaaacattagc

SEQ ID NO: 918 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccctgcggcaccagtcccaactgacgtttagcattgcgcttctccggaaacattagc

SEQ ID NO: 919 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcattaacgcacgggcaatcattaaacggccattgcgcttctccggaaacattagc

SEQ ID NO: 920 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcacattgagcggcgaagttcaatatccacgccattgcgcttctccggaaacattagc

SEQ ID NO: 921 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctctcgagatcgtaaccaaatacgctgaccccattgcgcttctccggaaacattagc

SEQ ID NO: 922 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgatgtacgcttctttgcgctccacgccgtacattgcgcttctccggaaacattagc

SEQ ID NO: 923 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccggtgcgagatcgagaataaaggtttccgacattgcgcttctccggaaacattagc

SEQ ID NO: 924 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccaccagctcaccgtgttgaataatgccgacattgcgcttctccggaaacattagc

SEQ ID NO: 925 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctcacccgcttcgacctgcaaatctatcccacattgcgcttctccggaaacattagc

SEQ ID NO: 926 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgatgacgcaacgttggcgtaggcattggtcattgcgcttctccggaaacattagc

SEQ ID NO: 927 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcttagctacgcaaaccacgtccacgttggatcattgcgcttctccggaaacattagc

SEQ ID NO: 928 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcgcgtctgcacccagatacgcataaagcgatcattgcgcttctccggaaacattagc

SEQ ID NO: 929 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcaaagtaaagggtcatggtgatgactggcggcattgcgcttctccggaaacattagc

SEQ ID NO: 930 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcggcaaacacaccgttcagcaaacccgcaagcattgcgcttctccggaaacattagc

SEQ ID NO: 931 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctacgtgagtggcgttaacacaaaggttggccattgcgcttctccggaaacattagc

SEQ ID NO: 932 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcctgcatatagctgaagccatgcatatcgcccattgcgcttctccggaaacattagc

SEQ ID NO: 933 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctataaacgattgggttcaggtgcgacagcccattgcgcttctccggaaacattagc

SEQ ID NO: 934 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctatccggcaataatgacgtgagtcggaacccattgcgcttctccggaaacattagc

SEQ ID NO: 935 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgcccgtgagcaccagcgttaaggcaacgaatacattgcgcttctccggaaacattagc

SEQ ID NO: 936 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgctgaccagcggaacatcattgataccgaggacattgcgcttctccggaaacattagc

SEQ ID NO: 937 - DNA, 102 bp, synthetic construct
  misc_feature 1..102: note = Synthetic Polynucleotide
  source 1..102: mol_type = other DNA
acaaccgcgtgttacaaggccaggcatccgagaggtctgggaatgccgccacaataaagaccaccagtacgccaaacattgcgcttctccggaaacattagc

(19) United States
(12) Patent Application Publication
(10) Pub. No.: US 2025/0179473 A1
(43) Pub. Date: Jun. 5, 2025

(54) PROBE LIBRARY CONSTRUCTION

(71) Applicant: President and Fellows of Harvard College, Cambridge, MA (US)

(72) Inventors: Xiaowei Zhuang, Cambridge, MA (US); Jeffrey R. Moffitt, Cambridge, MA (US); Alistair Boettiger, Cambridge, MA (US)

(73) Assignee: President and Fellows of Harvard College, Cambridge, MA (US)

(21) Appl. No.: 18/915,641

(22) Filed: Oct. 15, 2024

Related U.S. Application Data
(63) Continuation of application No. 16/253,919, filed on Jan. 22, 2019, now abandoned.
Application No. 16/253,919 is a continuation of application No. 15/329,651, filed on Jan. 27, 2017, now Pat. No. 10,240,146.
(60) Provisional application No. 62/142,653, filed on Apr. 3, 2015.
(60) Provisional application No. 62/050,636, filed on Sep. 15, 2014.
(60) Provisional application No. 62/031,062, filed on Jul. 30, 2014.

(51) Int. Cl. C12N 15/10; C07H 21/02; C07H 21/04; C12Q 1/6806; C12Q 1/6816; C12Q 1/6837; C12Q 1/6841; C12Q 1/6869; G06N 7/01; G16B 25/00; G16B 25/20; G16B 30/00; G16B 40/00; G16B 40/10; G16C 20/10

(52) U.S. Cl. CPC C12N 15/1065; C07H 21/02; C07H 21/04; C12N 15/10; C12N 15/1093; C12Q 1/6806; C12Q 1/6837; G16B 25/00; G16B 25/20; G16B 40/10; G16C 20/10; C12Q 1/6816; C12Q 1/6841; C12Q 1/6869; G06N 7/01; G16B 30/00; G16B 40/00

(57) ABSTRACT

The present invention generally relates to systems and methods for producing nucleic acids. In some aspects, relatively large quantities of oligonucleotides can be produced, and in some cases, the oligonucleotides may have a variety of different sequences and/or lengths. For instance, a relatively small quantity of oligonucleotides may be amplified to produce a large amount of nucleotides. In one set of embodiments, oligonucleotides may be amplified using PCR, then transcribed to produce RNA. The RNA may then be reverse transcribed to produce DNA, and optionally, the RNA may be selectively degraded or removed, relative to the DNA. In one set of embodiments, the oligonucleotides may be chemically modified. These modifications may include, but are not limited, to the adding of fluorescent dyes or other signaling entities.

### RELATED APPLICATIONS

This application is a continuation of and claims priority under 35 U.S.C. § 120 to U.S. patent application U.S. Ser. No. 16/253,919, filed Jan. 22, 2019, which is a continuation of U.S. patent application Ser. No. 15/329,651, filed Jan. 27, 2017, entitled “Probe Library Construction,” by Zhuang, et al., which is a national stage filing of International Patent Application Serial No. PCT/US2015/042559, entitled “Probe Library Construction,” by Zhuang, et al., which claims the benefit of U.S. Provisional Patent Application Ser. No. 62/050,636, filed Sep. 15, 2014, entitled “Probe Library Construction,” by Zhuang, et al.; U.S. Provisional Patent Application Ser. No. 62/031,062, filed Jul. 30, 2014, entitled “Systems and Methods for Determining Nucleic Acids,” by Zhuang, et al.; and U.S. Provisional Patent Application Ser. No. 62/142,653, filed Apr. 3, 2015, entitled “Systems and Methods for Determining Nucleic Acids,” by Zhuang, et al. Each of the above is incorporated herein by reference.

### GOVERNMENT FUNDING

This invention was made with government support under Grant No. GM096450 awarded by the National Institutes of Health. The government has certain rights in the invention.

### REFERENCE TO AN ELECTRONIC SEQUENCE LISTING

The contents of the electronic sequence listing (H049870510US03-SEQ-TC.xml; Size: 1,255,295 bytes; and Date of Creation: Oct. 8, 2024) are herein incorporated by reference in its entirety.

### FIELD

The present invention generally relates to systems and methods for producing nucleic acids.

### BACKGROUND

Custom-synthesized, oligonucleotide probes have emerged as a powerful tool for the identification and isolation of specific nucleic acid targets via hybridization. Applications for such hybridization probe sets range from next generation sequencing—where such probes are used to enrich or deplete samples for specific nucleic acid targets—to imaging of fixed samples—where fluorescently labeled hybridization probes allow the direct measurement of the number and spatial organization of the targeted species.

There are now a wide range of commercial sources for such probes. Such probes are often made by synthesizing each oligonucleotide member using standard solid phase synthesis methods. Unfortunately, this limits both the number of probes within a single set and the number of unique sets, due to the requirement that each oligonucleotide member must be individually and separately synthesized.

Recent advances in array-based synthesis of oligonucleotides by several companies have reduced the cost of producing oligonucleotides. However, these approaches also result in 1000-fold less oligonucleotide probes than is required for a single hybridization reaction, thus limiting their usefulness. Accordingly, improvements in oligonucleotide production are needed.

### SUMMARY

The present invention generally relates to systems and methods for producing nucleic acids. The subject matter of the present invention involves, in some cases, interrelated products, alternative solutions to a particular problem, and/or a plurality of different uses of one or more systems and/or articles.

In one aspect, the present invention is generally directed to a method. The method, in accordance with one set of embodiments, includes amplifying at least some of a plurality of oligonucleotides using real-time PCR to produce amplified oligonucleotides, transcribing in vitro at least some of the amplified oligonucleotides to produce RNA, reverse transcribing the RNA to produce transcribed DNA, and selectively degrading the RNA relative to the transcribed DNA.

In another set of embodiments, the method includes simultaneously amplifying at least some of a plurality of oligonucleotides in a common solution using PCR to produce amplified oligonucleotides, transcribing in vitro at least some of the amplified oligonucleotides to produce RNA, reverse transcribing the RNA to produce transcribed DNA, and selectively degrading the RNA relative to the transcribed DNA.

In yet another set of embodiments, the method includes acts of providing a plurality of oligonucleotides having an average length of between 10 and 200 nucleotides and including at least 100 unique oligonucleotide sequences, producing amplified oligonucleotides comprising one of the plurality of oligonucleotides and a promoter using real-time PCR, transcribing at least some of the amplified oligonucleotides to produce RNA using an RNA polymerase, reverse transcribing the RNA to produce DNA using a primer comprising a signaling entity, and chemically reducing the RNA.

The method, in still another set of embodiments, includes acts of providing a plurality of oligonucleotides having an average length of between 10 and 200 nucleotides and including at least 100 unique oligonucleotide sequences, producing amplified oligonucleotides in a common solution comprising one of the plurality of oligonucleotides and a promoter using PCR, transcribing at least some of the amplified oligonucleotides to produce RNA using an RNA polymerase, reverse transcribing the RNA to produce DNA using a primer comprising a signaling entity, and chemically reducing the RNA.

In another aspect, the present invention encompasses methods of making one or more of the embodiments described herein, such as oligonucleotides, including but not limited to modified oligonucleotides such as those described herein (e.g., labeled with a signaling entity). In still another aspect, the present invention encompasses methods of using one or more of the embodiments described herein, for example, such as oligonucleotides, including but not limited to modified oligonucleotides such as those described herein (e.g., labeled with a signaling entity).

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates the production of DNA probes, in accordance with one set of embodiments; and

FIG. 2 illustrates a template molecule (SEQ ID NO: 3) produced in accordance with one embodiment of the invention;

FIGS. 3A-3D list optimized primers from the E. coli transcriptome, in another embodiment of the invention (the sequences, from top to bottom and left to right, by page, correspond to SEQ ID NOs: 4-57, 58-111, 112-165, and 166-201); and

FIGS. 4A-4BV show various probes in accordance with yet another embodiment of the invention. The sequences in FIGS. 4A-4BV, from top to bottom and left to right, by page, correspond to SEQ ID NOs: 202-221, 222-243, 244-265, 266-287, 288-309, 310-331, 332-353, 354-375, 376-397, 398-419, 420-441, 442-463, 464-485, 486-507, 508-529, 530-551, 552-573, 574-595, 596-617, 618-639, 640-661, 662-683, 684-705, 706-727, 728-749, 750-771, 772-793, 794-815, 816-837, 838-859, 860-881, 882-903, 904-925, and 926-937.

### DETAILED DESCRIPTION

The present invention generally relates to systems and methods for producing nucleic acids. In some aspects, relatively large quantities of oligonucleotides can be produced, and in some cases, the oligonucleotides may have a variety of different sequences and/or lengths. For instance, a relatively small quantity of oligonucleotides may be amplified to produce a large amount of nucleotides. In one set of embodiments, oligonucleotides may be amplified using PCR, then transcribed to produce RNA. The RNA may then be reverse transcribed to produce DNA, and optionally, the RNA may be selectively degraded or removed, relative to the DNA. In one set of embodiments, the oligonucleotides may be chemically modified. These modifications may include, but are not limited, to the adding of fluorescent dyes or other signaling entities.

U.S. Provisional Patent Application Ser. No. 62/031,062, filed Jul. 30, 2014, entitled “Systems and Methods for Determining Nucleic Acids,” by Zhuang, et al. is incorporated herein by reference in its entirety.

In one aspect, the present invention is generally directed to in vitro methods of amplifying a plurality of oligonucleotides. In some cases, relatively large numbers of unique oligonucelotides within a plurality of oligonucleotides may be amplified. For instance, a plurality of oligonucleotides to be amplified may include 10, 100, 1,000, or more unique sequences.

In addition, in some embodiments, the oligonucleotides may be amplified without selective amplification of some oligonucleotides over others, e.g., due to competitive effects. Although some drift may occur, it is desired that the relative ratios of the oligonucleotides within a plurality of oligonucleotides stay substantially the same after amplification, at least for some applications. However, in many amplification techniques, due to differences in binding or affinity of different oligonucleotides, some oligonucleotides may be amplified to a greater degree than others, and thus, specific techniques need to be utilized to reduce or eliminate this problem, for example, by separately amplifying each of the oligonucleotides before combining them together to form the plurality of oligonucleotides. In contrast, as is discussed herein, in certain embodiments, a plurality of oligonucleotides can be amplified without causing substantial alterations or changes in the ratios of the oligonucleotides, without requiring separation of the oligonucleotides, separate growth of the oligonucleotides, or other cumbersome techniques.

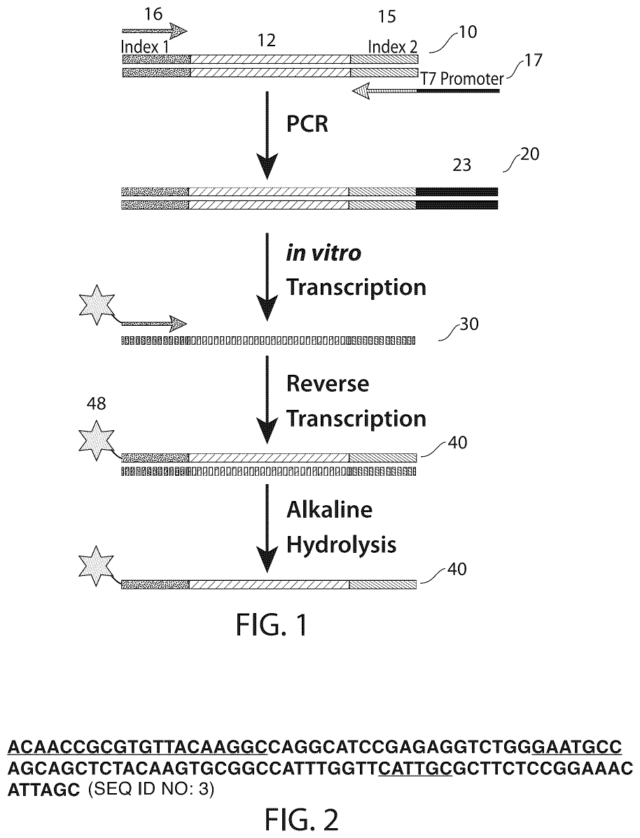

Referring now to FIG. 1, one example of an embodiment of the invention is now illustrated. In this figure, a plurality of oligonucleotides 10 is provided. This may include 1, 10, 100, 1,000, 10,000, 100,000, or any other suitable number of unique oligonucleotide sequences. Of course, more than one copy of any particular unique oligonucleotide may also be present as well within the plurality of oligonucleotides. The unique oligonucleotides may have the same or different lengths. In some cases, the plurality of oligonucleotides have an overall average length (number average or arithmetic mean) of less than 200 nt (nucleotides), although longer average lengths are also possible in some embodiments.

The plurality of oligonucleotides 10 may initially be amplified, using PCR (polymerase chain reaction) or another suitable oligonucleotide amplification method, to produce a plurality of amplified oligonucleotides 20. In some cases, PCR may be used to generate thousands to millions of copies per oligonucleotide within the plurality of oligonucleotides. In some embodiments, the plurality of oligonucleotides may be amplified while still contained in a common solution, for instance, without requiring separation of the oligonucleotides prior to amplification, e.g., as is required in certain techniques such as emulsion PCR.

Within a common solution, while it is possible that different oligonucleotides of the plurality of oligonucleotides may be amplified at different rates (e.g., leading to non-uniform amplification of the plurality of oligonucleotides, and the potential loss of complexity or species within the plurality of oligonucleotides during amplification), in certain embodiments, this can be reduced or minimized through the use of various oligonucleotide structures and/or through the use of certain types of PCR techniques, as is discussed herein.

As an example, in one set of embodiments, the plurality of oligonucleotides may all be chosen to minimize competitive effects, e.g., as caused by differences in binding or affinity of the oligonucleotides to reagents within the common solution or the preferential enzymatic amplification of some sequence features. For example, in one set of embodiments, the plurality of oligonucleotides may be chosen to have similar lengths and/or sequences.

As is shown in FIG. 1, the plurality of oligonucleotides may each contain one or more index regions 15, 16 on one or both ends of the oligonucleotides that can be recognized by certain reagents. In some cases, the oligonucleotides of the plurality of oligonucleotides may have one or more index regions to which suitable primers can interact with in order to allow PCR or other amplification to occur. In some embodiments, these index regions can be used to selectively produce DNA probes only from subset of the plurality of oligonucleotides 10.

These index regions can also be used in some instances to add additional sequences to that of the plurality of oligonucleotides, e.g., as is shown in FIG. 1, oligonucleotide 11 may include an index region 15, to which a sequence 17 containing a T7 promoter can bind and be introduced into the amplified oligonucleotides (e.g., as region 23). Various sequences may thus be applied to the plurality of oligonucleotides that include a portion able to bind an index region. In some cases, if substantially all of the plurality of oligonucleotides contain similar or identical index regions, the relative affinities or binding to the index regions of the oligonucleotides by enzymes such as polymerases may be substantially similar or identical, which may allow for relatively uniform amplification to occur. The plurality of oligonucleotides may also contain other different regions that can be varied to produce a plurality of unique oligonucleotides, e.g., region 12. These regions can vary in terms of length and/or sequence, etc.

In some embodiments, the amount of amplification that occurs may be carefully controlled by monitoring the PCR amplification reaction, e.g., using techniques such as real-time PCR. This may occur, for example, using oligonucleotides having common index regions, substantially similar lengths and/or sequences, etc., including those previously discussed, or with other suitable pluralities of oligonucleotides. For instance, in some embodiments, the PCR reaction may be monitored by illuminating the solution containing the oligonucleotides with suitable light and determining the amount of fluorescence that is present, which can be related to the DNA present within the sample. Techniques for monitoring PCR reactions, such as real-time PCR methodologies, are known to those of ordinary skill in the art. The PCR reaction can also be controlled, in some embodiments, by controlling the amount and/or concentration of nucleotides and/or cofactors that are present.

After amplification, the plurality of amplified oligonucleotides 20 may be transcribed to produce a plurality of RNAs 30, as is shown in the example of FIG. 1. This may be performed, for example, by exposing the amplified oligonucleotides to a suitable RNA polymerase, such as T7 RNA polymerase, that can transcribe the oligonucleotides to produce corresponding RNA. The amount of RNA production can be controlled in some embodiments by controlling the amount and/or concentration of nucleotides and/or cofactors that are present as well as the duration of the in vitro transcription reaction.

The plurality of RNAs 30, may then be used to produce additional amounts of DNA 40, e.g., by using reverse transcription. For example a suitable enzyme, such as reverse transcriptase, may be used to perform the reverse transcription. In some cases, primers may be used to facilitate transcription, and in some embodiments, the primers may also be used to attach additional entities to the DNA. For example, signaling entities may be attached, as is shown with signaling entity 48 in FIG. 1. Alternatively, additional nucleic acid sequences can also be attached, which can serve to recruit additional oligonucleotides via Watson-Crick base-pairing. The amount of DNA that is produced can be controlled, for example, by controlling the amount and/or concentration of nucleotides and/or cofactors that are present as well as the duration and temperature of the reverse transcription reaction.

In some embodiments, multiple copies of DNA may be produced from each RNA molecule. In addition, optionally, the RNA may then be removed or selectively degraded, relative to the DNA, for example, through alkaline hydrolysis, enzymatic digestion, or other techniques.

Accordingly, in certain aspects, the present invention is generally directed to systems and methods of amplifying a plurality of oligonucleotides. In one set of embodiments, relatively large quantities or masses of oligonucleotides can be produced as is discussed herein, e.g., at least about 10⁻³ pmol, at least about 10⁻² pmol, at least about 10⁻¹ pmol, at least about 10⁰ pmol, at least about 10¹ pmol, at least about 10² pmol, at least about 10³ pmol, etc. In addition, in some embodiments, the plurality of oligonucleotides may be substantially diverse. For example, the plurality of oligonucleotides may include at least about 10¹, at least about 10², at least about 10³, at least about 104, at least about 105, or at least about 10⁶ unique sequences of oligonucleotides, even after amplification to the amounts discussed above. (It should also be noted that a plurality or population of oligonucleotides may include more than one copy of a given unique oligonucleotide sequence.) In contrast, certain prior art techniques are able to amplify large numbers of unique oligonucleotides, but to only small quantities or masses (e.g., to amounts of around 10⁻³ pmol or less), or are able to produce large quantities or masses of oligonucleotides, but only for 1 or a few unique sequences (e.g., less than 10 sequences).

As discussed, in certain embodiments, a plurality of oligonucleotides, which may include a plurality of unique sequences of oligonucleotides such as those described above, may be amplified without substantial selective amplification of some oligonucleotide sequences over others, e.g., due to competitive effects, unlike in many prior art techniques. Although some drift may occur during the amplification process, the drift may be relatively small. For example, in certain embodiments, the ratios or percentages of a representative unique oligonucleotide sequence, relative to the starting overall population, on the average, may change upon amplification by no more than about 10%, no more than about 8%, no more than about 6%, no more than about 5%, no more than about 4%, no more than about 3%, no more than about 2%, no more than about 1%, etc., relative to the starting ratio or percentage of the oligonucleotide sequence, prior to amplification. However, it should be noted that the oligonucleotide sequence itself, prior to any amplification, may also exhibit some variability, which is not included in the above numbers.

The unique oligonucleotides within a plurality of oligonucleotides may have the same or different lengths. If more than one unique oligonucleotide is present, then the unique oligonucleotides may independently have the same or different lengths. For example, in some cases, a plurality of oligonucleotides may have an average length (number average) of at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides. In some cases, the average length may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides. Combinations of any of these are also possible, e.g., the average length may be between 10 and 30 nucleotides, between 20 and 40 nucleotides, between 5 and 50 nucleotides, between 10 and 200 nucleotides, or between 25 and 35 nucleotides, between 10 and 300 nucleotides, etc.

In one set of embodiments, any suitable technique may be used to amplify the plurality of oligonucleotides. In some cases, for each oligonucleotide to be amplified, at least about 100, at least about 300, at least about 500, at least about 1,000, at least about 3,000, at least about 5,000, at least about 10,000, at least about 30,000, at least about 50,000 at least about 100,000, at least about 300,000, at least about 500,000, at least about 1,000,000 copies, at least about 3,000,000 copies, at least about 5,000,000 copies, at least about 10,000,000 copies, at least about 30,000,000 copies, at least about 50,000,000 copies, or at least about 100,000,000 copies of the oligonucleotide may be produced using any of the amplification techniques discussed herein (e.g., including PCR amplification, in vitro transcription etc.). As discussed, in some cases, the amplification may occur without substantial selective amplification of some oligonucleotide sequences over others.

Any suitable technique may be used to generate the plurality of oligonucleotides. For example, the plurality of oligonucleotides may be synthetically produced, grown within a cell, grown on a substrate (e.g., in an array), or the like. Techniques for producing oligonucleotides are known to those of ordinary skill in the art. The plurality of oligonucleotides may also be computationally designed in some embodiments.

In one embodiment, the oligonucleotides may be amplified using PCR (polymerase chain reaction). In some cases, the oligonucleotides may be amplified while contained in a common liquid or solution. This is to be contrasted with certain PCR techniques, such as emulsion PCR or digital PCR, which requires separation of the oligonucleotides, e.g., into separate compartments or droplets, prior to amplification so as to prevent relatively selective amplification of certain oligonucleotides from occurring. However, surprisingly, it has been found that such separation is not required, and other techniques (such as is described herein) may be used to prevent or reduce selective amplification while keeping the oligonucleotides together within a common solution.

As mentioned, in some cases, by using certain oligonucleotide structures and/or certain types of PCR techniques, the amount of selective amplification that may occur may be reduced or eliminated. For instance, in one set of embodiments, oligonucleotides are chosen to minimize competitive effects. For instance, the oligonucleotides may have substantially the same lengths, and/or share identical or similar portions or regions.

For example, in one set of embodiments, the oligonucleotides may have a distribution of lengths such that no more than about 10%, no more than about 5%, no more than about 3%, or no more than about 1% of the oligonucleotides has a length that is less than about 80% or greater than about 120%, less than about 90% or greater than about 110%, or less than about 95% or greater than about 105% of the overall average length of the plurality of nucleotides.

In another set of embodiments the oligonucleotides may share one or more regions, such as index regions, that are identical or substantially similar. The oligonucleotides sharing index or other regions may have substantially the same lengths, as discussed above, or different lengths. In some embodiments, the oligonucleotides may comprise at least two index regions that each are identical or substantially similar, surrounding a variable region having different nucleotide sequences, and optionally, different lengths. For example, the oligonucleotides may include, in sequence, a first region that is identical or substantially similar to the other oligonucleotides, a second region that is not identical, and optionally, a third region that is identical or substantially similar to the other oligonucleotides. In some embodiments, competition of oligonucleotides may be controlled by using oligonucleotides selected to reduce amplification bias. For instance, in some cases, groups of oligonucleotides that have similar compositions may be amplified together.

In some cases, the index regions may have a length of greater than 5, 7, 10, 12, 14, 16, 18, or 20 nucleotides, and/or have a length of less than 30, 28, 25, 22, 20, 18, 16, 14, 12, or 10 nucleotides. For instance, the regions that are identical or substantially similar may have a length of between 18 and 22 nucleotides. The regions may be identical, or differ by no more than 1, 2, 3, 4, or 5 nucleotides (consecutively or non-consecutively) within the region.

In certain embodiments, primer sequences may be added to facilitate the PCR reaction. For example, the primer sequence may include sequences substantially complementary to a region within the oligonucleotides, e.g., an index region. A variety of such sequences suitable for PCR or in vitro transcription may be readily obtained commercially.

In some embodiments, the primer sequence may also include other sequences, e.g., promoter sequences or other sequences that may be added to the oligonucleotide during PCR amplification, such as is shown in FIG. 1 with a T7 promoter. Accordingly, oligonucleotides comprising the original sequence and one or more promoter sequences may be produced in certain cases. Besides the T7 promoter, other suitable promoters that may be used include, but are not limited to, T3 promoters or SP6 promoters. Such promoters may be useful, for example, to facilitate transcription to produce RNA, as is discussed in more detail below. In addition, in some embodiments, more than one promoter may be added.

In one set of embodiments, more than one sequence containing a PCR primer may be used, e.g., to amplify different subsets of the plurality of oligonucleotides. If more than one primer-containing sequence is used, the PCR primers contained on each of them may be the same or different. Examples of suitable PCR primers include those described herein. Thus, for example, in one set of embodiments, for example, the plurality of oligonucleotides may include different subpools having different index regions or other regions as discussed above, which may be selectively amplified through the use of an appropriate sequence including a PCR primer. Any suitable number of subpools may be created for a plurality of oligonucleotides. For example, at least 1, 2, 4, 10, 20, 96, 100, or 192 subpools of the plurality oligonucleotides may be selective amplified, through the use of specific PCR primers.

Thus, in some cases, the oligonucleotides may be formed into “pools” or groups or sets of oligonucleotides within the plurality of oligonucleotides that share one or more common features, such as an index region or other identical sequence. For instance, the common feature in a group or set of oligonucleotides may have an identical sequence of nucleotides of at least 5, 7, 10, 12, 14, 16, 18, or 20 nucleotides, and/or less than 30, 28, 25, 22, 20, 18, 16, 14, 12, or 10 nucleotides. For instance, the common region that is identical or substantially similar in a group of oligonucleotides may have a length of between 18 and 22 nucleotides. The common regions may be identical, or differ by no more than 1, 2, 3, 4, or 5 nucleotides (consecutively or non-consecutively) within the region. In some embodiments, each group or pool may contain two (or more) unique index regions that are not used in any other pool, e.g., to reduce the contamination of off-target amplified products from the amplification products of another.

In certain embodiments, PCR amplification may be monitored, and controlled to reduce or minimize selective amplification. For example, in one set of embodiments, real-time PCR techniques may be used. In some embodiments, the extent of the PCR reaction may be monitored or controlled, for example, by illuminating the solution containing the oligonucleotides with suitable light and determining the amount of fluorescence that is present to monitor the PCR reaction. Accordingly, for example, the reaction conditions may be controlled such that the oligonucleotides react in conditions that minimize the amount of selective amplification, for example, by providing an excess of nucleotides, ions (e.g., Mg²⁺), enzyme, etc. Once the oligonucleotide concentrations have reached the point where competitive effects may start to occur, the reaction may be stopped before significant selective amplification begins.

After amplification as discussed above, the amplified oligonucleotides may then be transcribed to produce RNA. Further amplification may also occur in this step. For instance, in some cases, each oligonucleotide can be used to produce, on the average, at least about 50, at least about 100, at least about 300, at least about 500, at least about 1,000, at least about 3,000, at least about 5,000, at least about 10,000, at least about 30,000, at least about 50,000 at least about 100,000, at least about 300,000, at least about 500,000 or at least about 1,000,000 transcribed RNA molecules. In some cases, the mass of RNA that is produced may be at least about 10, at least about 20, at least about 30, at least about 50, at least about 100, at least about 200, at least about 300, or at least about 500 times the mass of the oligonucleotides. Thus, for example, one microgram of oligonucleotides may be converted into at least 10 micrograms, at least 30 micrograms, or at least 100 micrograms of RNA.

In one set of embodiments, transcription may occur in vitro by exposing the amplified oligonucleotides to a suitable RNA polymerase. A variety of RNA polymerases are available commercially, including T7, T3, or SP6 RNA polymerases. Other non-limiting examples of RNA polymerases include RNA polymerase I, RNA polymerase II, RNA polymerase III, RNA polymerase IV, or RNA polymerase V. The RNA polymerase may arise from any suitable source, e.g., bacteria, viruses, or eukaryotes. In some embodiments, more than one RNA polymerase may be used. In addition, as previously discussed, in some embodiments, the amplified oligonucleotides may include promoter sequences, such as one or more of T7, T3, or SP6 promoter sequences, that can be used to facilitate the transcription process. Those of ordinary skill in the art will be aware of suitable conditions for causing transcription in vitro using RNA polymerases.

In some embodiments, the total amplification bias may be reduced by changing the relative amount of amplification produced by the PCR and the in vitro transcription. For example, the PCR can be used to produce smaller amounts of DNA than are typically produced in a PCR, to reduce the amplification bias of this process. However, this reduced yield can be compensated in some cases by increasing the duration of the in vitro transcription reaction.

The RNA may, in turn, be reverse transcribed to produce DNA. In one set of embodiments, reverse transcription may occur by exposing the RNA to a suitable reverse transcriptase enzyme. In some cases, the reverse transcriptase may be a viral reverse transcriptase, e.g., M-MLV reverse transcriptase, AMV reverse transcriptase, or the like. A variety of reverse transcriptase enzymes are commercially available. Those of ordinary skill in the art will be aware of suitable conditions for causing reverse transcription to occur.

In certain embodiments, reverse transcription may be facilitated through the use of primer-containing sequences, e.g., containing primers for reverse transcription. In some cases, the primer-containing sequences may contain other sequences or entities as well, although this is not necessarily a requirement. The primer-containing sequences may be added at any suitable point, e.g., just before starting transcription reaction. Suitable transcription primers for conducting reverse transcription may be obtained commercially.

In one set of embodiments, the primer-containing sequence may be incorporated into the DNA during production of the DNA by the reverse transcriptase. In some cases, the primer-containing sequence may contain other entities, and/or sequences suitable for attaching other entities (e.g., on the 5′ or 3′ ends, internally, etc.). For instance, the primer-containing sequence may contain a non-nucleic acid moiety, such as a digoxigenin moiety, a biotin moiety, etc. located on the 5′ end, the 3′ end, internally, or the like. In some cases, the signaling entity that can be subsequently detected or determined may be introduced to the DNA. For instance, the signaling entity may be fluorescent, or a specific nucleotide sequence that can be determined, e.g., enzymatically. Examples of signaling entities are discussed in more detail below.

In some cases, the RNA may be purified prior to reverse transcription. However, it should be noted that purification is not required, and in other embodiments, the RNA may be reverse transcribed to form DNA without any intermediate purification steps. If the RNA is purified, it may be purified using any suitable technique, e.g., by passing the RNA over a suitable column to remove oligonucleotides.

Optionally, in some embodiments, the RNA may be separated from the DNA or the DNA may be purified in some fashion. For example, the RNA may be selectively degraded, relative to the DNA. In one set of embodiments, the RNA may be degraded relative to the DNA by alkaline hydrolysis. For instance, the pH of the solution may be raised to at least about 8, at least about 9, at least about 10, etc. Any suitable alkaline may be used to raise the pH. In some cases, after degradation of the RNA, the pH may also be lowered, e.g., to about 7, to about 7.4, to physiological conditions, or the like. In some cases, techniques such as enzymatic degradation can be used to selectively degrade RNA, relative to DNA. The DNA may also be purified using techniques such as column purification, ethanol precipitation, and/or solid-phase reversible immobilization techniques. In addition, in some cases, the DNA may be concentrated, e.g., through evaporation techniques.

In addition, techniques such as those described above may be scaled-up or “numbered-up” to produce larger quantities of material. For example, a process may be repeated using multi-well techniques or by simultaneously running multiple reactions in parallel, etc. to produce larger quantities or masses of oligonucleotides. As a non-limiting example, in one embodiment, processes such as those discussed herein may be performed using multiple wells of a microtiter plate (e.g., having 96, 384, 1536, wells, etc.) to increase output.

The DNA may be used for a variety of purposes, in different embodiments of the invention. For example, in certain embodiments, the DNA may be hybridized to nucleic acid species in liquid samples, e.g., extracted from a variety of biological sources, including human. In some cases, the DNA may be used to physically separate one set of nucleic acids from another, or as primers for PCR or reverse transcription.

In addition, as previously discussed, in certain aspects, signaling entities are incorporated into DNA in some embodiments. The signaling entities may be determined for a variety of purposes. For example, the DNA that is produced may be used as a biological probe, and the signaling entities may be determined in some fashion, e.g., quantitatively or qualitatively, to determine a characteristic or feature of the probe. Examples include, but are not limited to, the position of the probe, the activity of the probe, the concentration of the probe, or the like.

In some cases, signaling entities within a sample may be determined, e.g., spatially, using a variety of techniques. In some embodiments, the signaling entities may be fluorescent, and techniques for determining fluorescence within a sample, such as fluorescence microscopy or confocal microscopy, may be used to spatially identify the positions of signaling entities within a cell. In some cases, the positions of the entities within the sample may be determined in two or even three dimensions.

In some embodiments, the spatial positions of the signaling entities may be determined at relatively high resolutions. For instance, the positions may be determined at spatial resolutions of better than about 100 micrometers, better than about 30 micrometers, better than about 10 micrometers, better than about 3 micrometers, better than about 1 micrometer, better than about 800 nm, better than about 600 nm, better than about 500 nm, better than about 400 nm, better than about 300 nm, better than about 200 nm, better than about 100 nm, better than about 90 nm, better than about 80 nm, better than about 70 nm, better than about 60 nm, better than about 50 nm, better than about 40 nm, better than about 30 nm, better than about 20 nm, or better than about 10 nm, etc.

There are a variety of techniques able to determine or image the spatial positions of entities optically, e.g., using fluorescence microscopy. In some cases, the spatial positions may be determined at super resolutions, or at resolutions better than the wavelength of light. Non-limiting examples include STORM (stochastic optical reconstruction microscopy), STED (stimulated emission depletion microscopy), NSOM (Near-field Scanning Optical Microscopy), 4Pi microscopy, SIM (Structured Illumination Microscopy), SMI (Spatially Modulated Illumination) microscopy, RESOLFT (Reversible Saturable Optically Linear Fluorescence Transition Microscopy), GSD (Ground State Depletion Microscopy), SSIM (Saturated Structured-Illumination Microscopy), SPDM (Spectral Precision Distance Microscopy), Photo-Activated Localization Microscopy (PALM), Fluorescence Photoactivation Localization Microscopy (FPALM), LIMON (3D Light Microscopical Nanosizing Microscopy), Super-resolution optical fluctuation imaging (SOFI), or the like. See, e.g., U.S. Pat. No. 7,838,302, issued Nov. 23, 2010, entitled “Sub-Diffraction Limit Image Resolution and Other Imaging Techniques,” by Zhuang, et al.; U.S. Pat. No. 8,564,792, issued Oct. 22, 2013, entitled “Sub-diffraction Limit Image Resolution in Three Dimensions,” by Zhuang, et al.; or Int. Pat. Apl. Pub. No. WO 2013/090360, published Jun. 20, 2013, entitled “High Resolution Dual-Objective Microscopy,” by Zhuang, et al., each incorporated herein by reference in their entireties.

In addition, the signaling entity may be inactivated in some cases. For example, in some embodiments, a first secondary nucleic acid probe containing a signaling entity may be applied to a sample that can recognize a first read sequence, then the first secondary nucleic acid probe can be inactivated before a second secondary nucleic acid probe is applied to the sample. If multiple signaling entities are used, the same or different techniques may be used to inactivate the signaling entities, and some or all of the multiple signaling entities may be inactivated, e.g., sequentially or simultaneously.

Inactivation may be caused by removal of the signaling entity (e.g., from the sample, or from the nucleic acid probe, etc.), and/or by chemically altering the signaling entity in some fashion, e.g., by photobleaching the signaling entity, bleaching or chemically altering the structure of the signaling entity, etc.). For instance, in one set of embodiments, a fluorescent signaling entity may be inactivated by chemical or optical techniques such as oxidation, photobleaching, chemically bleaching, stringent washing or enzymatic digestion or reaction by exposure to an enzyme, dissociating the signaling entity from other components (e.g., a probe), chemical reaction of the signaling entity (e.g., to a reactant able to alter the structure of the signaling entity) or the like.

In some embodiments, various nucleic acid probes (including primary and/or secondary nucleic acid probes) may include one or more signaling entities. If more than one nucleic acid probe is used, the signaling entities may each by the same or different. In certain embodiments, a signaling entity is any entity able to emit light. For instance, in one embodiment, the signaling entity is fluorescent. In other embodiments, the signaling entity may be phosphorescent, radioactive, absorptive, etc. In some cases, the signaling entity is any entity that can be determined within a sample at relatively high resolutions, e.g., at resolutions better than the wavelength of visible light. The signaling entity may be, for example, a dye, a small molecule, a peptide or protein, or the like. The signaling entity may be a single molecule in some cases. If multiple secondary nucleic acid probes are used, the nucleic acid probes may comprise the same or different signaling entities.

Non-limiting examples of signaling entities include fluorescent entities (fluorophores) or phosphorescent entities, for example, cyanine dyes (e.g., Cy2, Cy3, Cy3B, Cy5, Cy5.5, Cy7, etc.), Alexa Fluor dyes, Atto dyes, photoswtichable dyes, photoactivatable dyes, fluorescent dyes, metal nanoparticles, semiconductor nanoparticles or “quantum dots”, fluorescent proteins such as GFP (Green Fluorescent Protein), or photoactivabale fluorescent proteins, such as PAGFP, PSCFP, PSCFP2, Dendra, Dendra2, EosFP, tdEos, mEos2, mEos3, PAmCherry, PAtagRFP, mMaple, mMaple2, and mMaple3. Other suitable signaling entities are known to those of ordinary skill in the art. See, e.g., U.S. Pat. No. 7,838,302 or U.S. Pat. Apl. Ser. No. 61/979,436, each incorporated herein by reference in its entirety.

As used herein, the term “light” generally refers to electromagnetic radiation, having any suitable wavelength (or equivalently, frequency). For instance, in some embodiments, the light may include wavelengths in the optical or visual range (for example, having a wavelength of between about 400 nm and about 700 nm, i.e., “visible light”), infrared wavelengths (for example, having a wavelength of between about 300 micrometers and 700 nm), ultraviolet wavelengths (for example, having a wavelength of between about 400 nm and about 10 nm), or the like. In certain cases, as discussed in detail below, more than one entity may be used, i.e., entities that are chemically different or distinct, for example, structurally. However, in other cases, the entities may be chemically identical or at least substantially chemically identical.

In one set of embodiments, the signaling entity is “switchable,” i.e., the entity can be switched between two or more states, at least one of which emits light having a desired wavelength. In the other state(s), the entity may emit no light, or emit light at a different wavelength. For instance, an entity may be “activated” to a first state able to produce light having a desired wavelength, and “deactivated” to a second state not able to emit light of the same wavelength. An entity is “photoactivatable” if it can be activated by incident light of a suitable wavelength. As a non-limiting example, Cy5, can be switched between a fluorescent and a dark state in a controlled and reversible manner by light of different wavelengths, i.e., 633 nm (or 642 nm, 647 nm, 656 nm) red light can switch or deactivate Cy5 to a stable dark state, while 405 nm green light can switch or activate the Cy5 back to the fluorescent state. In some cases, the entity can be reversibly switched between the two or more states, e.g., upon exposure to the proper stimuli. For example, a first stimuli (e.g., a first wavelength of light) may be used to activate the switchable entity, while a second stimuli (e.g., a second wavelength of light) may be used to deactivate the switchable entity, for instance, to a non-emitting state. Any suitable method may be used to activate the entity. For example, in one embodiment, incident light of a suitable wavelength may be used to activate the entity to emit light, i.e., the entity is “photoswitchable.” Thus, the photoswitchable entity can be switched between different light-emitting or non-emitting states by incident light, e.g., of different wavelengths. The light may be monochromatic (e.g., produced using a laser) or polychromatic. In another embodiment, the entity may be activated upon stimulation by electric field and/or magnetic field. In other embodiments, the entity may be activated upon exposure to a suitable chemical environment, e.g., by adjusting the pH, or inducing a reversible chemical reaction involving the entity, etc. Similarly, any suitable method may be used to deactivate the entity, and the methods of activating and deactivating the entity need not be the same. For instance, the entity may be deactivated upon exposure to incident light of a suitable wavelength, or the entity may be deactivated by waiting a sufficient time.

Typically, a “switchable” entity can be identified by one of ordinary skill in the art by determining conditions under which an entity in a first state can emit light when exposed to an excitation wavelength, switching the entity from the first state to the second state, e.g., upon exposure to light of a switching wavelength, then showing that the entity, while in the second state can no longer emit light (or emits light at a much reduced intensity) when exposed to the excitation wavelength.

In one set of embodiments, as discussed, a switchable entity may be switched upon exposure to light. In some cases, the light used to activate the switchable entity may come from an external source, e.g., a light source such as a laser light source, another light-emitting entity proximate the switchable entity, etc. The second, light emitting entity, in some cases, may be a fluorescent entity, and in certain embodiments, the second, light-emitting entity may itself also be a switchable entity.

In some embodiments, the switchable entity includes a first, light-emitting portion (e.g., a fluorophore), and a second portion that activates or “switches” the first portion. For example, upon exposure to light, the second portion of the switchable entity may activate the first portion, causing the first portion to emit light. Examples of activator portions include, but are not limited to, Alexa Fluor 405 (Invitrogen), Alexa Fluor 488 (Invitrogen), Cy2 (GE Healthcare), Cy3 (GE Healthcare), Cy3B (GE Healthcare), Cy3.5 (GE Healthcare), or other suitable dyes. Examples of light-emitting portions include, but are not limited to, Cy5, Cy5.5 (GE Healthcare), Cy7 (GE Healthcare), Alexa Fluor 647 (Invitrogen), Alexa Fluor 680 (Invitrogen), Alexa Fluor 700 (Invitrogen), Alexa Fluor 750 (Invitrogen), Alexa Fluor 790 (Invitrogen), DiD, DiR, YOYO-3 (Invitrogen), YO-PRO-3 (Invitrogen), TOT-3 (Invitrogen), TO-PRO-3 (Invitrogen) or other suitable dyes. These may linked together, e.g., covalently, for example, directly, or through a linker, e.g., forming compounds such as, but not limited to, Cy5-Alexa Fluor 405, Cy5-Alexa Fluor 488, Cy5-Cy2, Cy5-Cy3, Cy5-Cy3.5, Cy5.5-Alexa Fluor 405, Cy5.5-Alexa Fluor 488, Cy5.5-Cy2, Cy5.5-Cy3, Cy5.5-Cy3.5, Cy7-Alexa Fluor 405, Cy7-Alexa Fluor 488, Cy7-Cy2, Cy7-Cy3, Cy7-Cy3.5, Alexa Fluor 647-Alexa Fluor 405, Alexa Fluor 647-Alexa Fluor 488, Alexa Fluor 647-Cy2, Alexa Fluor 647-Cy3, Alexa Fluor 647-Cy3.5, Alexa Fluor 750-Alexa Fluor 405, Alexa Fluor 750-Alexa Fluor 488, Alexa Fluor 750-Cy2, Alexa Fluor 750-Cy3, or Alexa Fluor 750-Cy3.5. Those of ordinary skill in the art will be aware of the structures of these and other compounds, many of which are available commercially. The portions may be linked via a covalent bond, or by a linker, such as those described in detail below. Other light-emitting or activator portions may include portions having two quaternized nitrogen atoms joined by a polymethine chain, where each nitrogen is independently part of a heteroaromatic moiety, such as pyrrole, imidazole, thiazole, pyridine, quinoine, indole, benzothiazole, etc., or part of a nonaromatic amine. In some cases, there may be 5, 6, 7, 8, 9, or more carbon atoms between the two nitrogen atoms.

In certain cases, the light-emitting portion and the activator portions, when isolated from each other, may each be fluorophores, i.e., entities that can emit light of a certain, emission wavelength when exposed to a stimulus, for example, an excitation wavelength. However, when a switchable entity is formed that comprises the first fluorophore and the second fluorophore, the first fluorophore forms a first, light-emitting portion and the second fluorophore forms an activator portion that switches that activates or “switches” the first portion in response to a stimulus. For example, the switchable entity may comprise a first fluorophore directly bonded to the second fluorophore, or the first and second entity may be connected via a linker or a common entity. Whether a pair of light-emitting portion and activator portion produces a suitable switchable entity can be tested by methods known to those of ordinary skills in the art. For example, light of various wavelength can be used to stimulate the pair and emission light from the light-emitting portion can be measured to determined wither the pair makes a suitable switch.

As a non-limiting example, Cy3 and Cy5 may be linked together to form such an entity. In this example, Cy3 is an activator portion that is able to activate Cy5, the light-emission portion. Thus, light at or near the absorption maximum (e.g., near 532 nm light for Cy3) of the activation or second portion of the entity may cause that portion to activate the first, light-emitting portion, thereby causing the first portion to emit light (e.g., near 647 nm for Cy5). See, e.g., U.S. Pat. No. 7,838,302, incorporated herein by reference in its entirety. In some cases, the first, light-emitting portion can subsequently be deactivated by any suitable technique (e.g., by directing 647 nm red light to the Cy5 portion of the molecule).

Other non-limiting examples of potentially suitable activator portions include 1,5 IAEDANS, 1,8-ANS, 4-Methylumbelliferone, 5-carboxy-2,7-dichlorofluorescein, 5-Carboxyfluorescein (5-FAM), 5-Carboxynapthofluorescein, 5-Carboxytetramethylrhodamine (5-TAMRA), 5-FAM (5-Carboxyfluorescein), 5-HAT (Hydroxy Tryptamine), 5-Hydroxy Tryptamine (HAT), 5-ROX (carboxy-X-rhodamine), 5-TAMRA (5-Carboxytetramethylrhodamine), 6-Carboxyrhodamine 6G, 6-CR 6G, 6-JOE, 7-Amino-4-methylcoumarin, 7-Aminoactinomycin D (7-AAD), 7-Hydroxy-4-methylcoumarin, 9-Amino-6-chloro-2-methoxyacridine, ABQ, Acid Fuchsin, ACMA (9-Amino-6-chloro-2-methoxyacridine), Acridine Orange, Acridine Red, Acridine Yellow, Acriflavin, Acriflavin Feulgen SITSA, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alizarin Complexon, Alizarin Red, AMC, AMCA-S, AMCA (Aminomethylcoumarin), AMCA-X, Aminoactinomycin D, Aminocoumarin, Aminomethylcoumarin (AMCA), Anilin Blue, Anthrocyl stearate, APTRA-BTC, APTS, Astrazon Brilliant Red 4G, Astrazon Orange R, Astrazon Red 6B, Astrazon Yellow 7 GLL, Atabrine, ATTO 390, ATTO 425, ATTO 465, ATTO 488, ATTO 495, ATTO 520, ATTO 532, ATTO 550, ATTO 565, ATTO 590, ATTO 594, ATTO 610, ATTO 611X, ATTO 620, ATTO 633, ATTO 635, ATTO 647, ATTO 647N, ATTO 655, ATTO 680, ATTO 700, ATTO 725, ATTO 740, ATTO-TAG CBQCA, ATTO-TAG FQ, Auramine, Aurophosphine G, Aurophosphine, BAO 9 (Bisaminophenyloxadiazole), BCECF (high pH), BCECF (low pH), Berberine Sulphate, Bimane, Bisbenzamide, Bisbenzimide (Hoechst), bis-BTC, Blancophor FFG, Blancophor SV, BOBO-1, BOBO-3, Bodipy 492/515, Bodipy 493/503, Bodipy 500/510, Bodipy 505/515, Bodipy 530/550, Bodipy 542/563, Bodipy 558/568, Bodipy 564/570, Bodipy 576/589, Bodipy 581/591, Bodipy 630/650-X, Bodipy 650/665-X, Bodipy 665/676, Bodipy Fl, Bodipy FL ATP, Bodipy Fl-Ceramide, Bodipy R6G, Bodipy TMR, Bodipy TMR-X conjugate, Bodipy TMR-X, SE, Bodipy TR, Bodipy TR ATP, Bodipy TR-X SE, BO-PRO-1, BO-PRO-3, Brilliant Sulphoflavin FF, BTC, BTC-5N, Calcein, Calcein Blue, Calcium Crimson, Calcium Green, Calcium Green-1 Ca²⁺ Dye, Calcium Green-2 Ca²⁺, Calcium Green-5N Ca²⁺, Calcium Green-C18 Ca2+, Calcium Orange, Calcofluor White, Carboxy-X-rhodamine (5-ROX), Cascade Blue, Cascade Yellow, Catecholamine, CCF2 (GeneBlazer), CFDA, Chromomycin A, Chromomycin A, CL-NERF, CMFDA, Coumarin Phalloidin, CPM Methylcoumarin, CTC, CTC Formazan, Cy2, Cy3.1 8, Cy3.5, Cy3, Cy5.1 8, cyclic AMP Fluorosensor (FiCRhR), Dabcyl, Dansyl, Dansyl Amine, Dansyl Cadaverine, Dansyl Chloride, Dansyl DHPE, Dansyl fluoride, DAPI, Dapoxyl, Dapoxyl 2, Dapoxyl 3′ DCFDA, DCFH (Dichlorodihydrofluorescein Diacetate), DDAO, DHR (Dihydorhodamine 123), Di-4-ANEPPS, Di-8-ANEPPS (non-ratio), DiA (4-Di-16-ASP), Dichlorodihydrofluorescein Diacetate (DCFH), DiD—Lipophilic Tracer, DiD (DiIC18(5)), DIDS, Dihydorhodamine 123 (DHR), DiI (DiIC18(3)), Dinitrophenol, DiO (DiOC18(3)), DiR, DiR (DiIC18(7)), DM-NERF (high pH), DNP, Dopamine, DTAF, DY-630-NHS, DY-635-NHS, DyLight 405, DyLight 488, DyLight 549, DyLight 633, DyLight 649, DyLight 680, DyLight 800, ELF 97, Eosin, Erythrosin, Erythrosin ITC, Ethidium Bromide, Ethidium homodimer-1 (EthD-1), Euchrysin, EukoLight, Europium (III) chloride, Fast Blue, FDA, Feulgen (Pararosaniline), FIF (Formaldehyd Induced Fluorescence), FITC, Flazo Orange, Fluo-3, Fluo-4, Fluorescein (FITC), Fluorescein Diacetate, Fluoro-Emerald, Fluoro-Gold (Hydroxystilbamidine), Fluor-Ruby, FluorX, FM 1-43, FM 4-46, Fura Red (high pH), Fura Red/Fluo-3, Fura-2, Fura-2/BCECF, Genacryl Brilliant Red B, Genacryl Brilliant Yellow 10GF, Genacryl Pink 3G, Genacryl Yellow 5GF, GeneBlazer (CCF2), Gloxalic Acid, Granular blue, Haematoporphyrin, Hoechst 33258, Hoechst 33342, Hoechst 34580, HPTS, Hydroxycoumarin, Hydroxystilbamidine (FluoroGold), Hydroxytryptamine, Indo-1, high calcium, Indo-1, low calcium, Indodicarbocyanine (DiD), Indotricarbocyanine (DiR), Intrawhite Cf, JC-1, JO-JO-1, JO-PRO-1, LaserPro, Laurodan, LDS 751 (DNA), LDS 751 (RNA), Leucophor PAF, Leucophor SF, Leucophor WS, Lissamine Rhodamine, Lissamine Rhodamine B, Calcein/Ethidium homodimer, LOLO-1, LO-PRO-1, Lucifer Yellow, Lyso Tracker Blue, Lyso Tracker Blue-White, Lyso Tracker Green, Lyso Tracker Red, Lyso Tracker Yellow, LysoSensor Blue, LysoSensor Green, LysoSensor Yellow/Blue, Mag Green, Magdala Red (Phloxin B), Mag-Fura Red, Mag-Fura-2, Mag-Fura-5, Mag-Indo-1, Magnesium Green, Magnesium Orange, Malachite Green, Marina Blue, Maxilon Brilliant Flavin 10 GFF, Maxilon Brilliant Flavin 8 GFF, Merocyanin, Methoxycoumarin, Mitotracker Green FM, Mitotracker Orange, Mitotracker Red, Mitramycin, Monobromobimane, Monobromobimane (mBBr-GSH), Monochlorobimane, MPS (Methyl Green Pyronine Stilbene), NBD, NBD Amine, Nile Red, Nitrobenzoxadidole, Noradrenaline, Nuclear Fast Red, Nuclear Yellow, Nylosan Brilliant Iavin E8G, Oregon Green, Oregon Green 488-X, Oregon Green, Oregon Green 488, Oregon Green 500, Oregon Green 514, Pacific Blue, Pararosaniline (Feulgen), PBFI, Phloxin B (Magdala Red), Phorwite AR, Phorwite BKL, Phorwite Rev, Phorwite RPA, Phosphine 3R, PKH26 (Sigma), PKH67, PMIA, Pontochrome Blue Black, POPO-1, POPO-3, PO-PRO-1, PO-PRO-3, Primuline, Procion Yellow, Propidium Iodid (PI), PyMPO, Pyrene, Pyronine, Pyronine B, Pyrozal Brilliant Flavin 7GF, QSY 7, Quinacrine Mustard, Resorufin, RH 414, Rhod-2, Rhodamine, Rhodamine 110, Rhodamine 123, Rhodamine 5 GLD, Rhodamine 6G, Rhodamine B, Rhodamine B 200, Rhodamine B extra, Rhodamine BB, Rhodamine BG, Rhodamine Green, Rhodamine Phallicidine, Rhodamine Phalloidine, Rhodamine Red, Rhodamine WT, Rose Bengal, S65A, S65C, S65L, S65T, SBFI, Serotonin, Sevron Brilliant Red 2B, Sevron Brilliant Red 4G, Sevron Brilliant Red B, Sevron Orange, Sevron Yellow L, SITS, SITS (Primuline), SITS (Stilbene Isothiosulphonic Acid), SNAFL calcein, SNAFL-1, SNAFL-2, SNARF calcein, SNARFI, Sodium Green, SpectrumAqua, SpectrumGreen, SpectrumOrange, Spectrum Red, SPQ (6-methoxy-N-(3-sulfopropyl)quinolinium), Stilbene, Sulphorhodamine B can C, Sulphorhodamine Extra, SYTO 11, SYTO 12, SYTO 13, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Green, SYTOX Orange, Tetracycline, Tetramethylrhodamine (TAMRA), Texas Red, Texas Red-X conjugate, Thiadicarbocyanine (DiSC3), Thiazine Red R, Thiazole Orange, Thioflavin 5, Thioflavin S, Thioflavin TCN, Thiolyte, Thiozole Orange, Tinopol CBS (Calcofluor White), TMR, TO-PRO-1, TO-PRO-3, TO-PRO-5, TOTO-1, TOTO-3, TRITC (tetramethylrodamine isothiocyanate), True Blue, TruRed, Ultralite, Uranine B, Uvitex SFC, WW 781, X-Rhodamine, XRITC, Xylene Orange, Y66F, Y66H, Y66W, YO-PRO-1, YO-PRO-3, YOYO-1, YOYO-3, SYBR Green, Thiazole orange (interchelating dyes), or combinations thereof.

In some aspects, the nucleotides can be used to study a sample, such as a biological sample. For instance, the nucleotides may be used to determine nucleic acids within a cell or other sample. The sample may include a cell culture, a suspension of cells, a biological tissue, a biopsy, an organism, or the like. The sample may also be cell-free but nevertheless contain nucleic acids. If the sample contains a cell, the cell may be a human cell, or any other suitable cell, e.g., a mammalian cell, a fish cell, an insect cell, a plant cell, or the like. More than one cell may be present in some cases.

The nucleic acids to be determined may be, for example, DNA, RNA, or other nucleic acids that are present within a cell (or other sample). The nucleic acids may be endogenous to the cell, or added to the cell. For instance, the nucleic acid may be viral, or artificially created. In some cases, the nucleic acid to be determined may be expressed by the cell. The nucleic acid is RNA in some embodiments. The RNA may be coding and/or non-coding RNA. Non-limiting examples of RNA that may be studied within the cell include mRNA, siRNA, rRNA, miRNA, tRNA, lncRNA, snoRNAs, snRNAs, exRNAs, piRNAs, or the like. In some embodiments, for example, at least some of the plurality of oligonucleotides are complementary to a portion of a specific chromosome sequence, e.g., of a human chromosome.

In some cases, a significant portion of the nucleic acid within the cell may be studied. For instance, in some cases, enough of the RNA present within a cell may be determined so as to produce a partial or complete transcriptome of the cell. In some cases, at least 4 types of mRNAs are determined within a cell, and in some cases, at least 3, at least 4, at least 7, at least 8, at least 12, at least 14, at least 15, at least 16, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000 types of mRNAs may be determined within a cell.

In some cases, the transcriptome of a cell may be determined. It should be understood that the transcriptome generally encompasses all RNA molecules produced within a cell, not just mRNA. Thus, for instance, the transcriptome may also include rRNA, tRNA, etc. In some embodiments, at least 5%, at least 10%, at least 15%, at least 20%, at least 25%, at least 30%, at least 40%, at least 50%, at least 60%, at least 70%, at least 80%, at least 90%, or 100% of the transcriptome of a cell may be determined.

The determination of one or more nucleic acids within the cell or other sample may be qualitative and/or quantitative. In addition, the determination may also be spatial, e.g., the position of the nucleic acid within the cell or other sample may be determined in two or three dimensions. In some embodiments, the positions, number, and/or concentrations of nucleic acids within the cell (or other sample) may be determined.

One non-limiting example of such as system may be found in U.S. Provisional Patent Application Ser. No. 62/031,062, filed Jul. 30, 2014, entitled “Systems and Methods for Determining Nucleic Acids,” by Zhuang, et al. incorporated herein by reference in its entirety.

The following documents are each incorporated herein by reference in their entireties: U.S. Pat. No. 7,838,302, issued Nov. 23, 2010, entitled “Sub-Diffraction Limit Image Resolution and Other Imaging Techniques,” by Zhuang, et al.; U.S. Pat. No. 8,564,792, issued Oct. 22, 2013, entitled “Sub-diffraction Limit Image Resolution in Three Dimensions,” by Zhuang, et al.; and Int. Pat. Apl. Pub. No. WO 2013/090360, published Jun. 20, 2013, entitled “High Resolution Dual-Objective Microscopy,” by Zhuang, et al. In addition, incorporated herein by reference in their entireties are U.S. Provisional Patent Application Ser. No. 62/031,062, filed Jul. 30, 2014, entitled “Systems and Methods for Determining Nucleic Acids,” by Zhuang, et al.; U.S. Provisional Patent Application Ser. No. 62/050,636, filed Sep. 15, 2014, entitled “Probe Library Construction,” by Zhuang, et al.; U.S. Provisional Patent Application Ser. No. 62/142,653, filed Apr. 3, 2015, entitled “Systems and Methods for Determining Nucleic Acids,” by Zhuang, et al.; and a PCT application filed on even date herewith, entitled “Systems and Methods for Determining Nucleic Acids,” by Zhuang, et al.

The following examples are intended to illustrate certain embodiments of the present invention, but do not exemplify the full scope of the invention.

### Example 1

This example illustrates high-throughput hybridization construction of DNA probes, according to certain embodiments of the invention.

Overview. This protocol uses complex libraries of oligonucleotides as templates for the enzymatic construction of large quantities of single-stranded DNA molecules that can be chemically labeled and which are designed to be hybridized to specific sets of nucleic acid targets that can vary significantly in complexity, i.e. the number of unique target sequences. The use of this protocol involves several basic steps: 1) computational design and optimization of a set of oligonucleotide sequences that will serve as the hybridization regions on the sample of interest; 2) computational design and optimization of a large set of short oligonucleotide sequences to serve as highly specific PCR primers; 3) computational construction of the template molecules; 4) synthesis of the template molecules to create the template library; 5) selection of a sub-set of in vitro template molecules from a template library via PCR; 6) in-vitro-transcription-based amplification of the in vitro template molecules into RNA, which serves as the final template for the hybridization probes; 7) reverse transcription of the RNA back into DNA using chemically-modified primers; 8) removal of the RNA via alkaline hydrolysis; and 9) purification of the chemically-modified ssDNA molecules, called probes in this example. These steps are discussed in detail below.

Construction of Probes. Selection of Hybridization Regions. The software OligoArray 2.0 was used to design a large set of potential hybridization regions in the E. coli transcriptome in this example. Briefly, this software selects hybridization regions that lie within user-specified ranges for length and melting temperature. This software also screens for off-target hybridization as well as potential secondary structure. This software was used to generate hybridization regions of 30-nt length, a melting-temperature range of 80-85° C., a GC content of between 50-60%, and a secondary structure melting threshold and cross-hybridization-melting temperature of 75° C. using all annotated, transcribed mRNAs in E. coli (K-12 mg1655; NC90013.2).

Design of Hundreds of Orthogonal Primers. The general hybridization probe set may require far fewer unique sequences than is provided in the complex sets of oligonucleotides typically generated by array-based synthesis—the oligopool. To exploit this disparity in complexity and significantly lower the cost per experiment, this example uses a method to embed a large number of unique template sets within a single oligopool. Briefly, each template molecule was flanked by a unique pair of PCR primers common only to the template molecules for all probes within a given set. To facilitate the embedding of hundreds of unique probe template sets within a single oligopool, a protocol was created for constructing hundreds of orthogonal PCR primers.

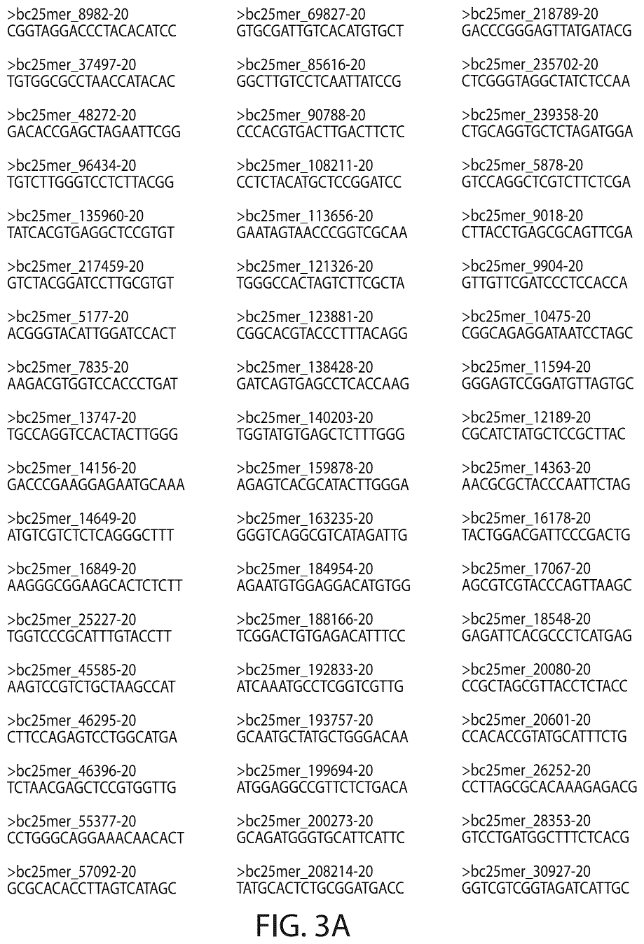

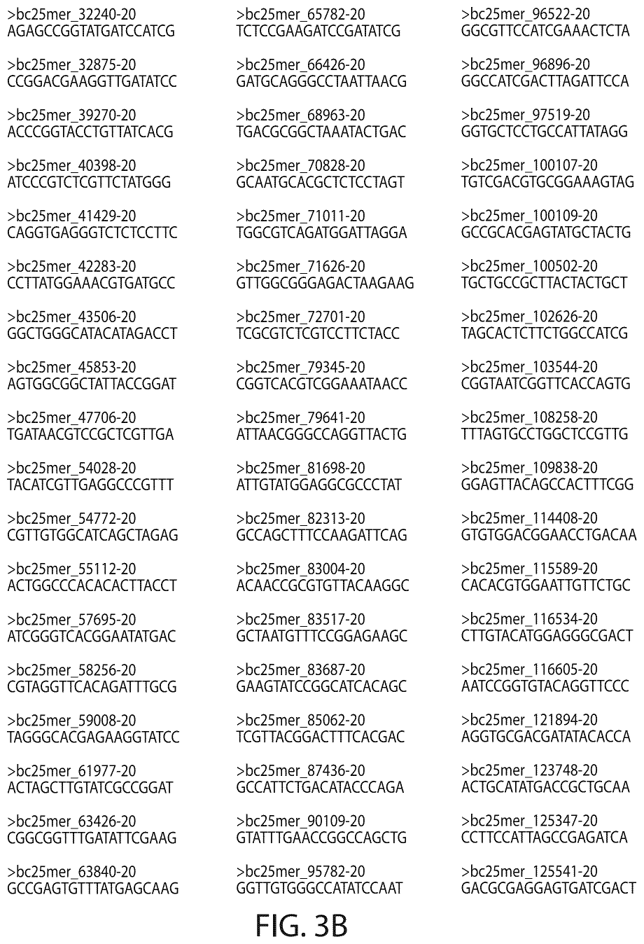

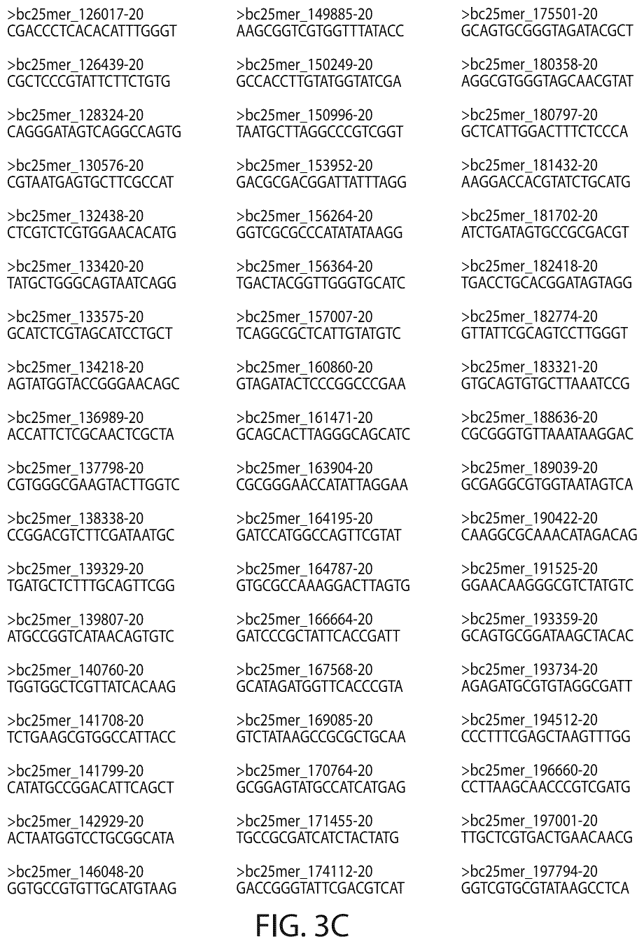

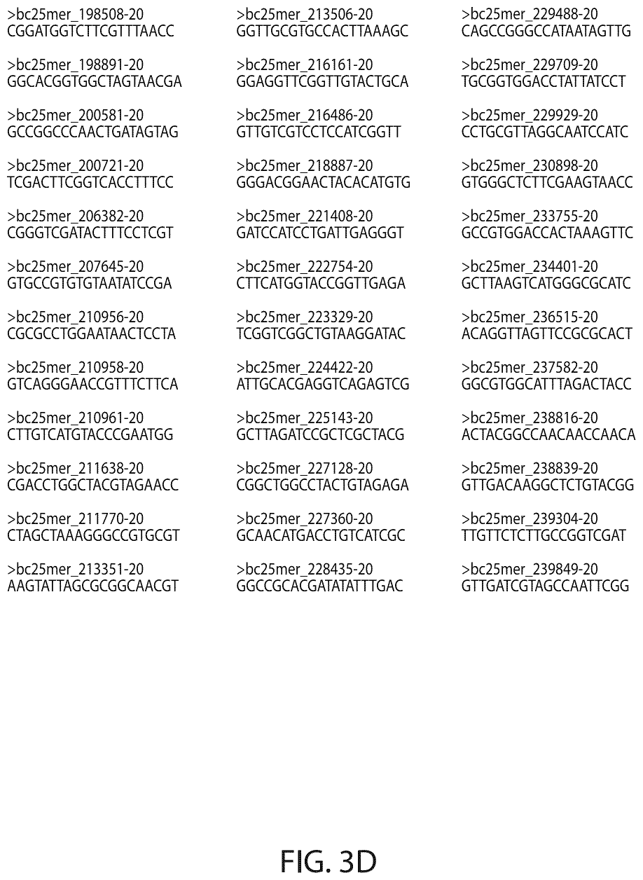

Specifically, the protocol starts by truncating the members of an existing library of 240,000, semi-orthogonal, 25-mer oligonucleotides to 20-nt length and selecting oligos on parameters optimal for PCR: a narrow range of predicted melting-temperature (65-70° C.) and GC content (50-60%); the absence of contiguous runs of the same base longer than 4, i.e. AAAA; and the presence of a 3′ GC-clamp, i.e. 2-3 G/C within the final 5 nt. BLAST was then run with these optimized primers against all annotated RNAs in the E. coli transcriptome as well as the T7 promoter (TAATACGACTCACTATAGGG) (SEQ ID NO. 1) and a common priming region (P9: CAGGCATCCGAGAGGTCTGG) (SEQ ID NO. 2) and potential primers with hits to the transcriptome with 12-nt or longer of homology, or with a hit of any length within 3-nt of the 3′ end of the potential primer, were removed. Finally, the remaining primers were screened for homology to each other, again using BLAST. Any primer with longer than 11-nt of homology or with any homology within 3-nt of the 3′ end of another primer was removed. These cuts reduced the original 240,000 oligos to 198 highly optimized primers for the E. coli transcriptome. The final set of 198 primers are listed in FIGS. 3A-3D.

If required, more primers could be generated using established techniques to create a larger set of initial oligonucleotides, or by relaxing the stringency of the cuts described above. Finally, by changing the transcriptome used to screen the primers, this approach can also be generalized to the generation of optimal index primers for any organism.

Template Construction. To design the template libraries used to create our probe sets, the desired RNA targets were first selected to be stained simultaneously. Individual template molecules were designed by concatenating the following sequences: i) the first of two unique primers for the appropriate mRNA group, ii) the common primer P9, iii) the site for the nicking enzyme Nb.BsmI, iv) the reverse complement of the hybridization region to the target, v) the reverse complement of the nicking enzyme Nb.BsrDI, and vi) the reverse complement of the second unique primer for the given mRNA group. FIG. 2 demonstrates this organization. Multiple probe template sets were combined into large oligopools and these pools were synthesized via CustomArray.

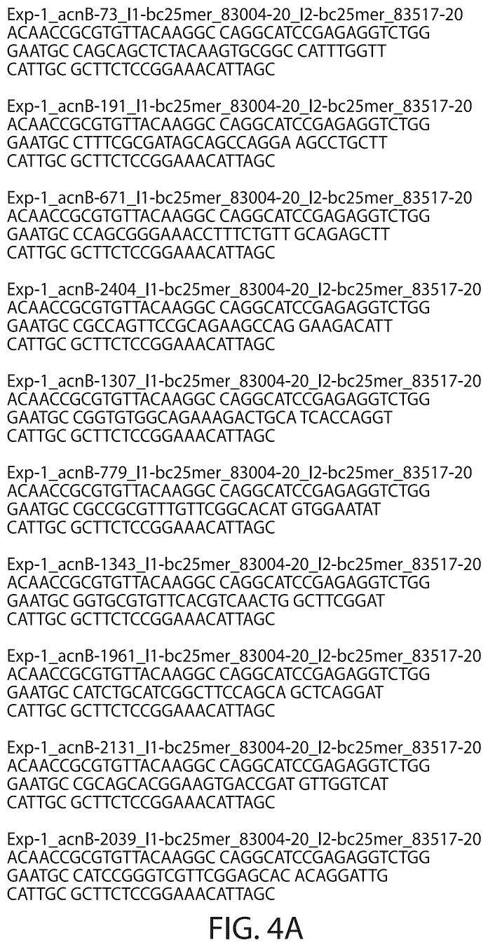

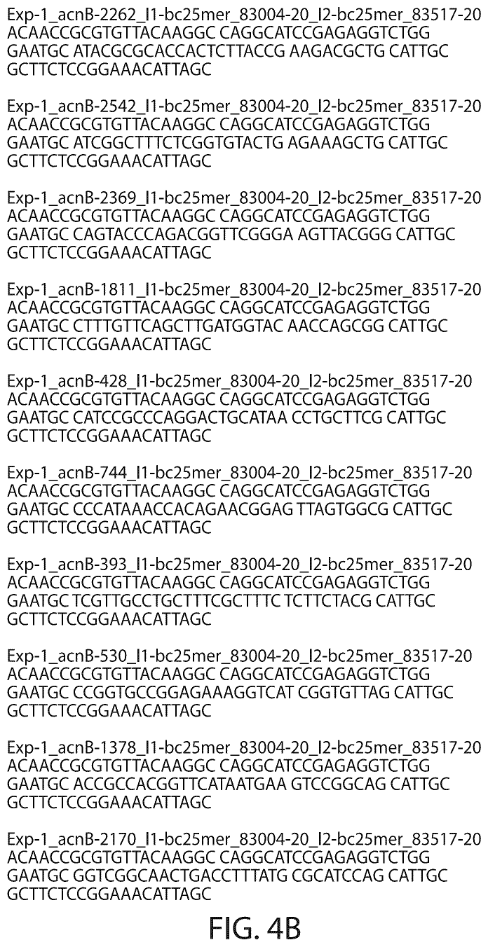

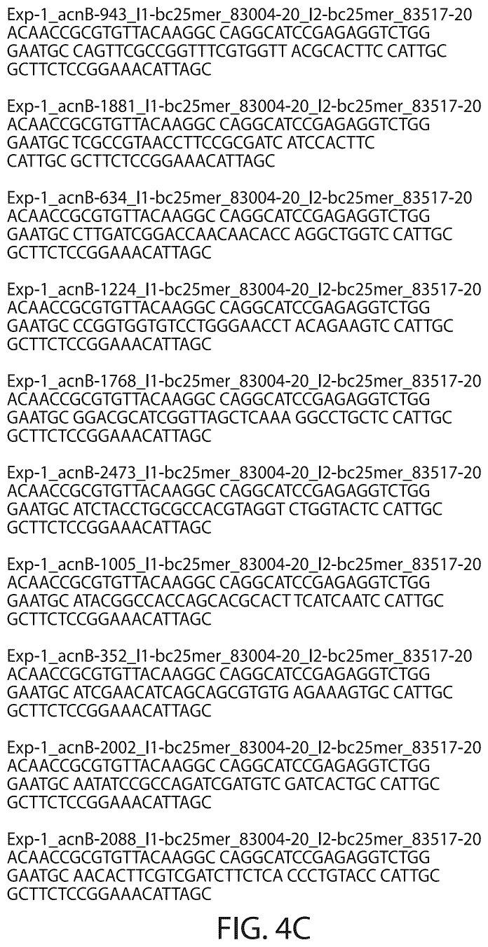

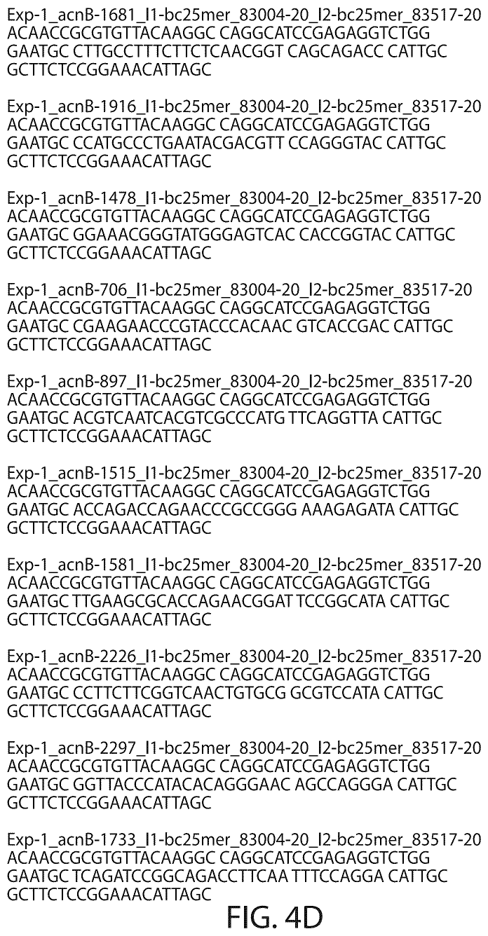

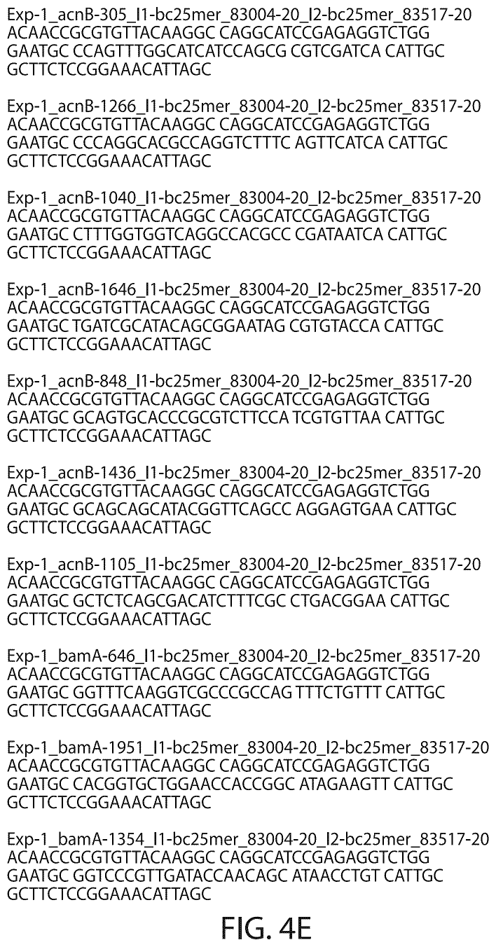

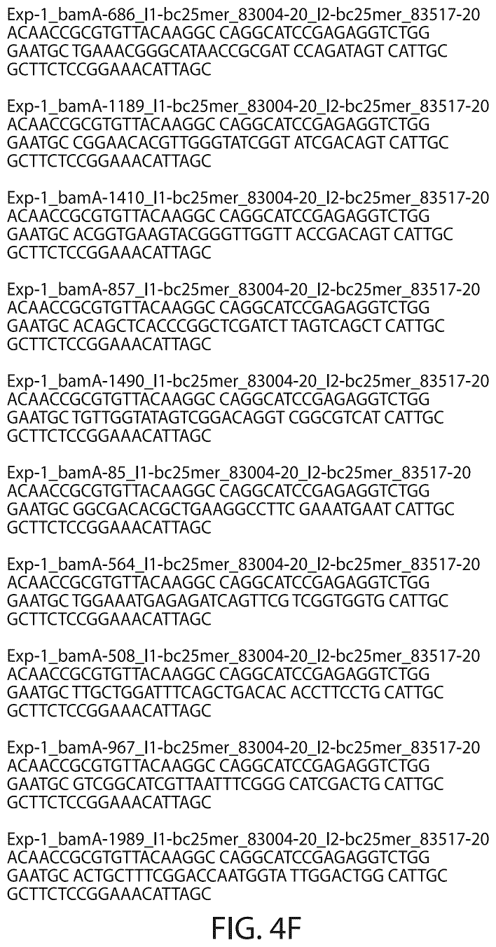

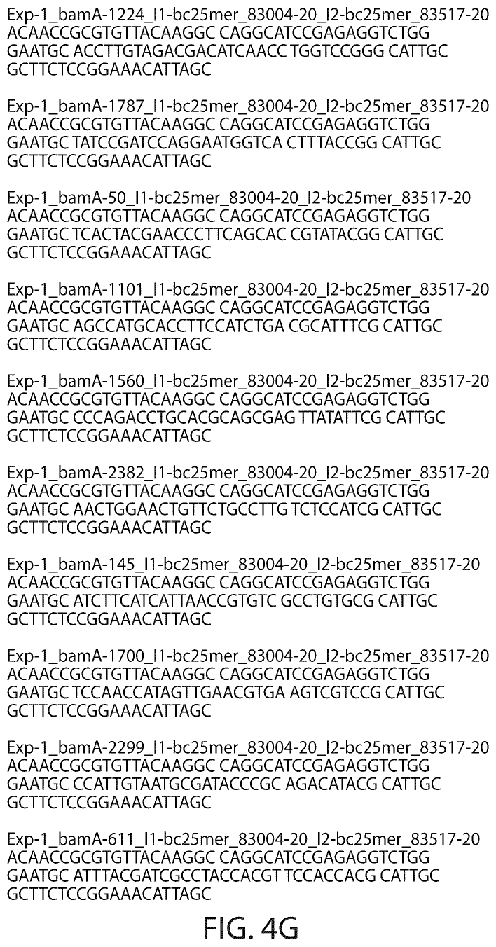

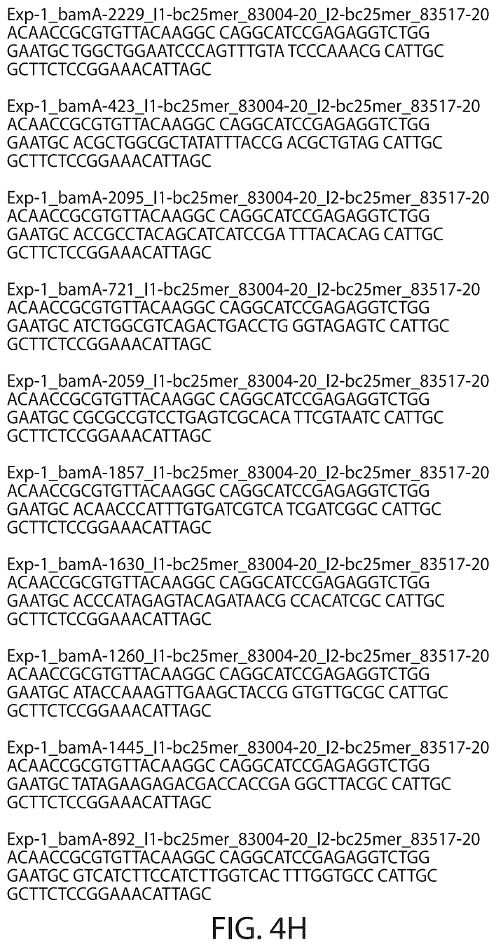

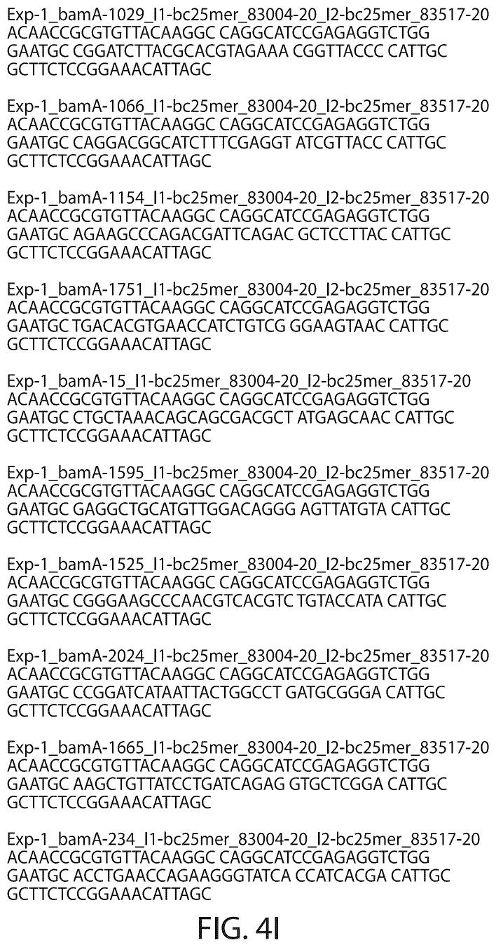

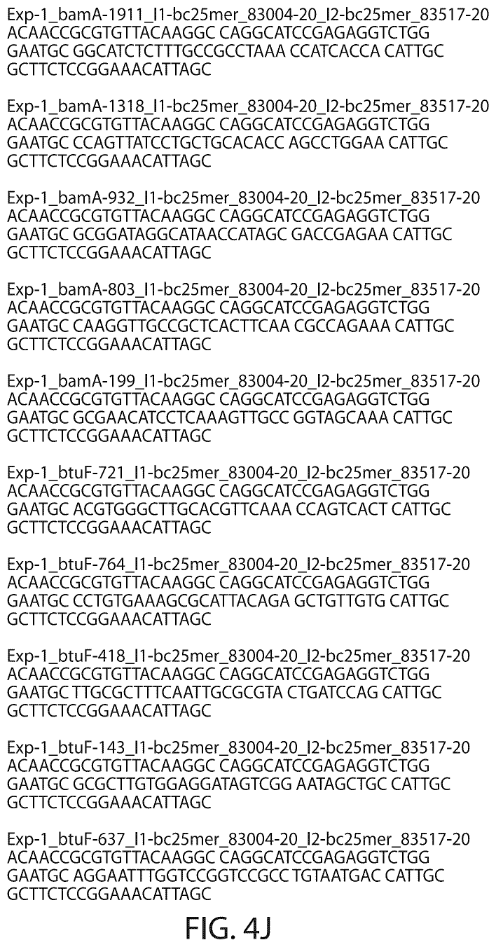

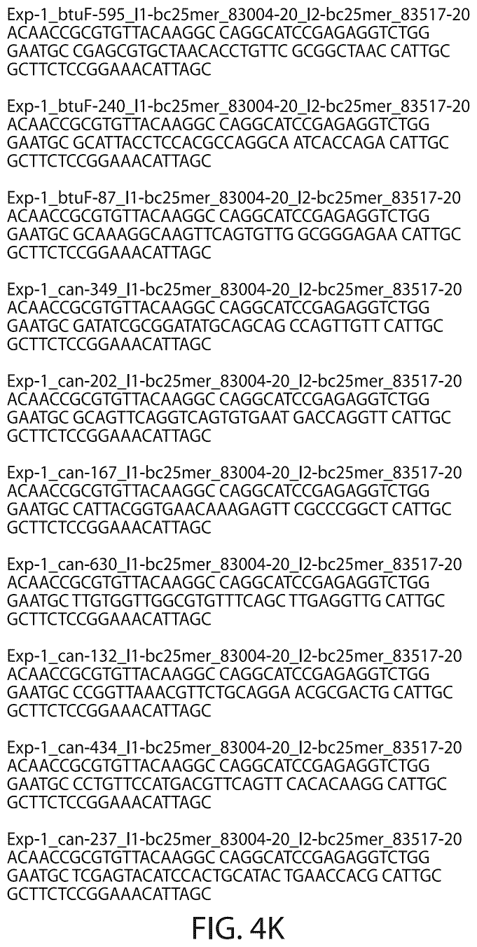

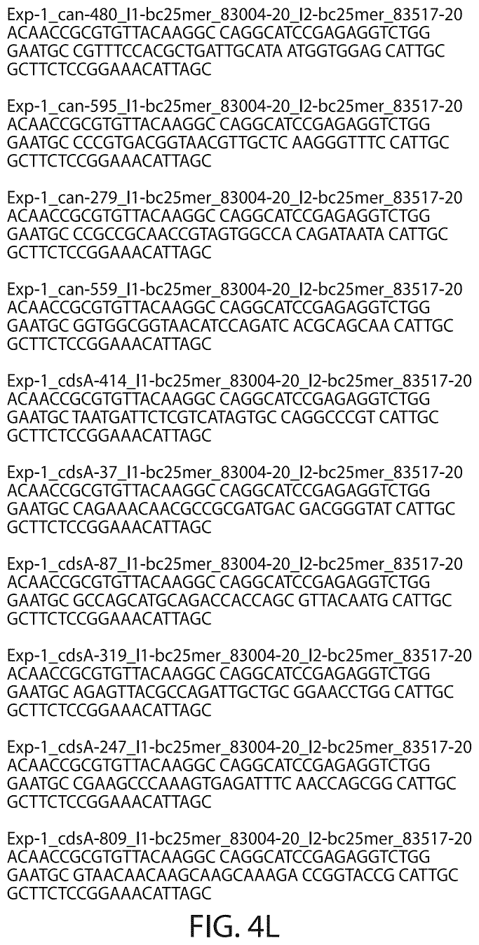

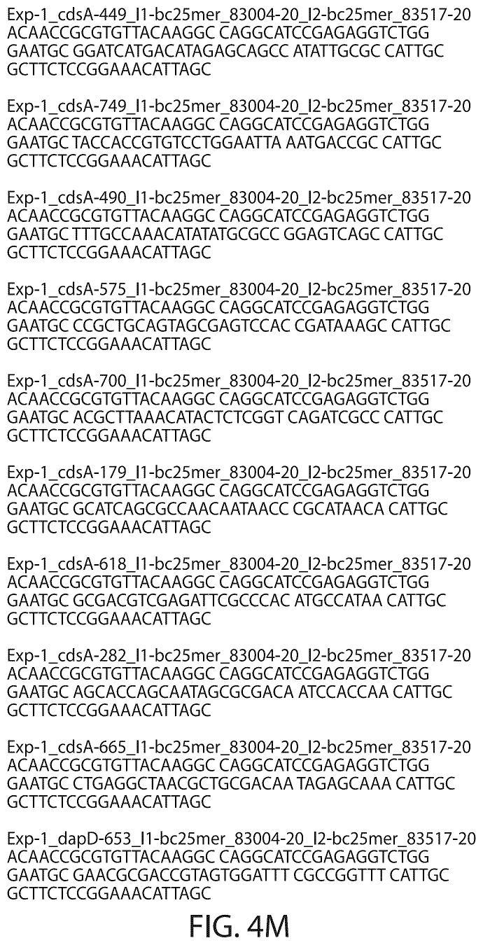

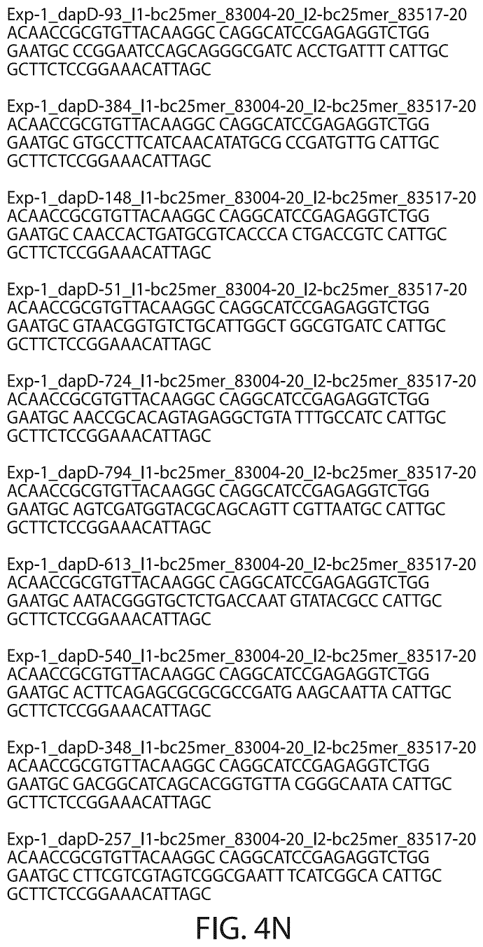

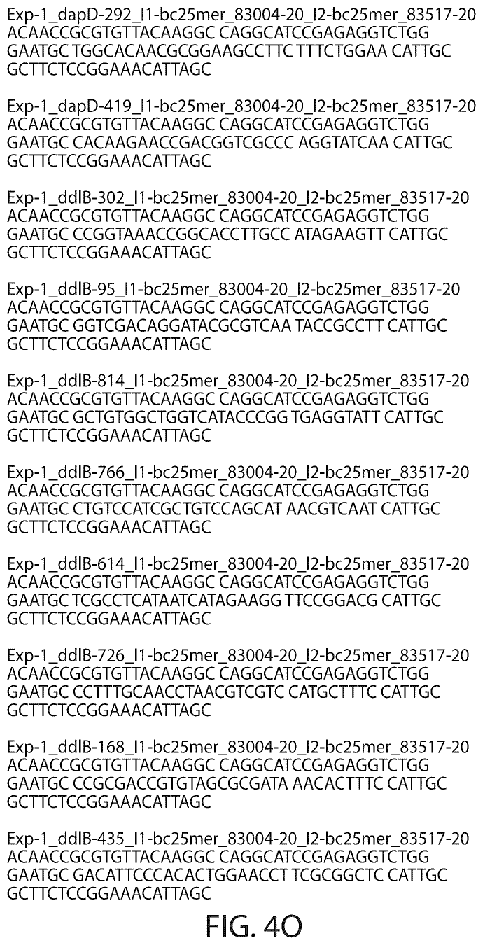

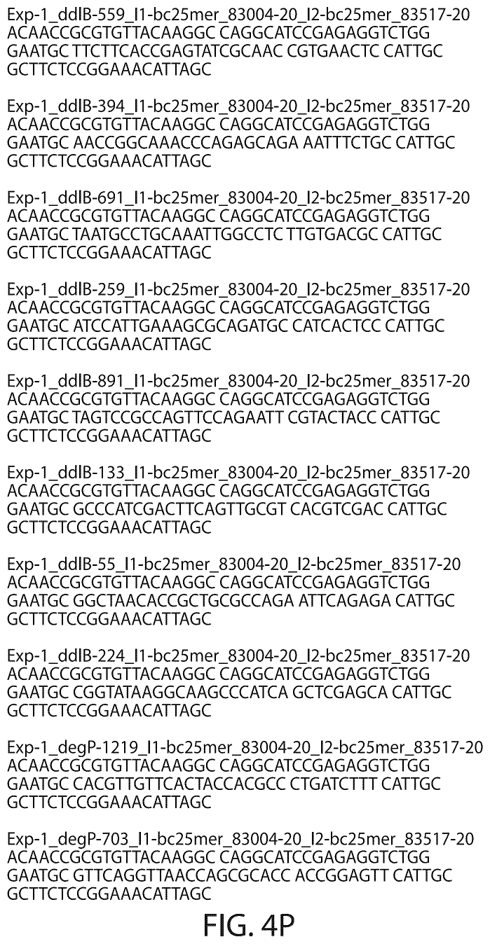

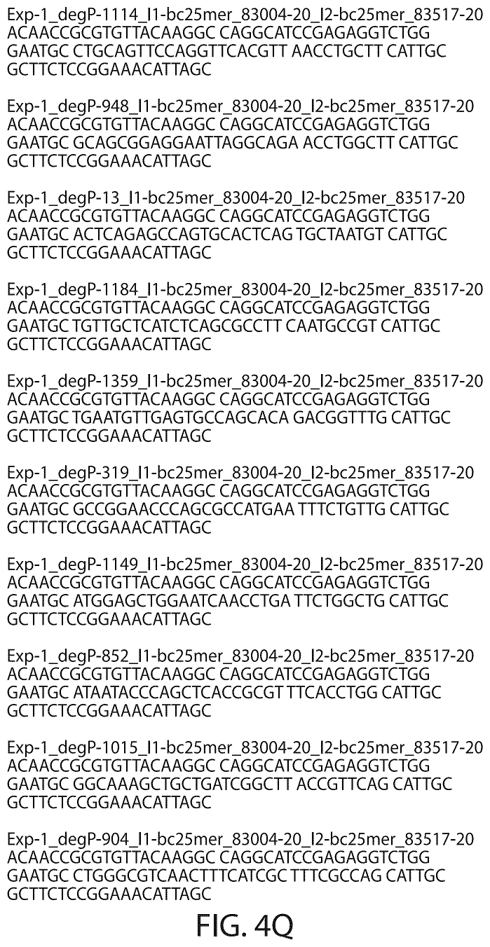

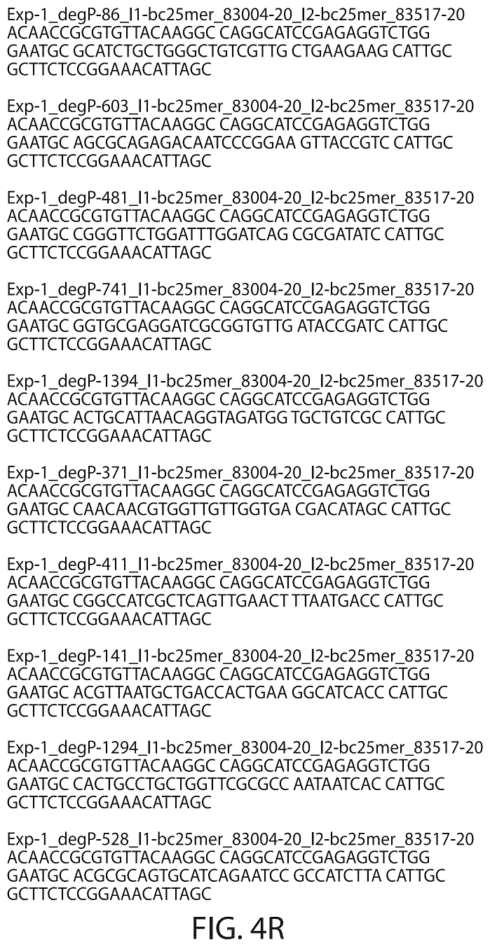

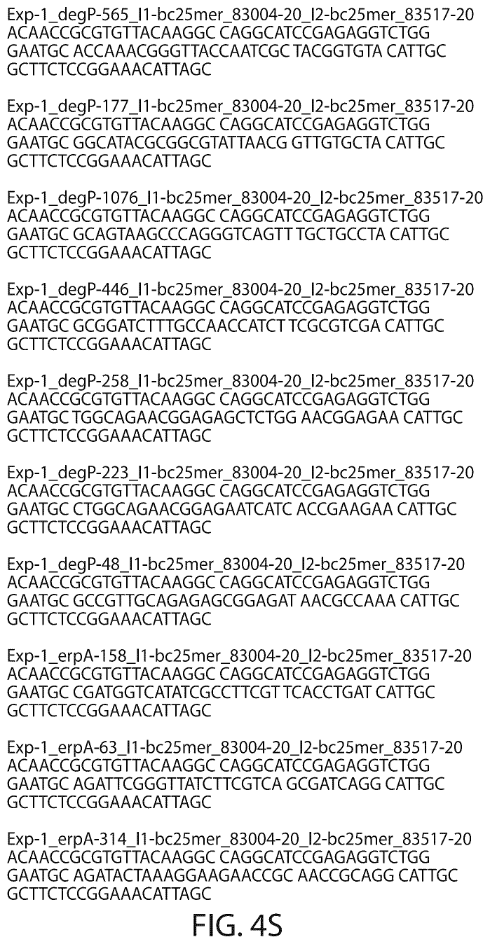

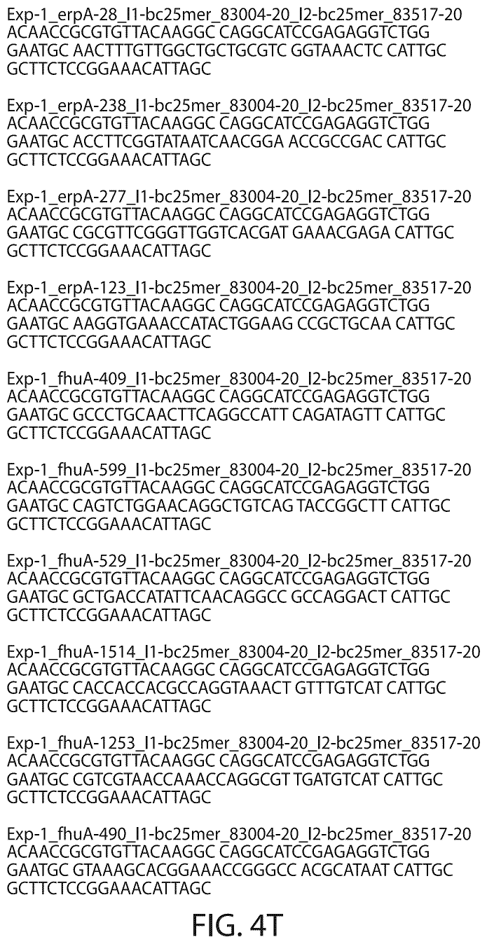

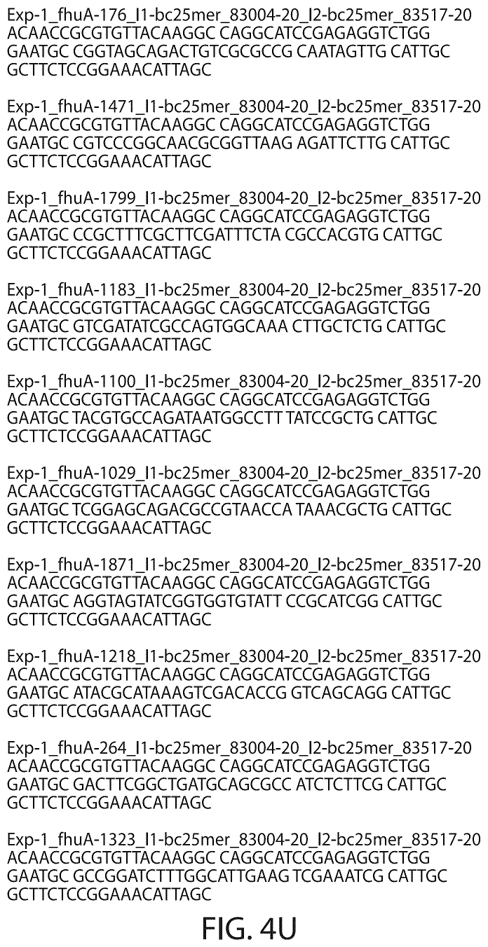

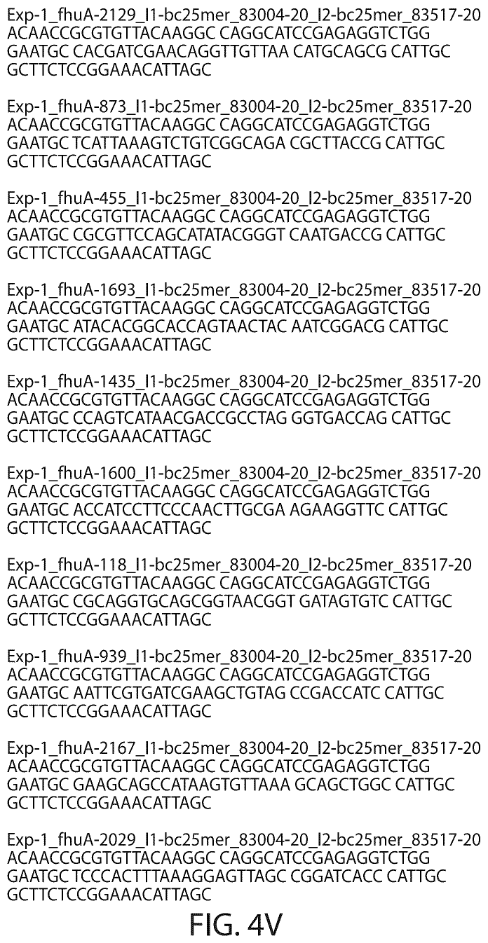

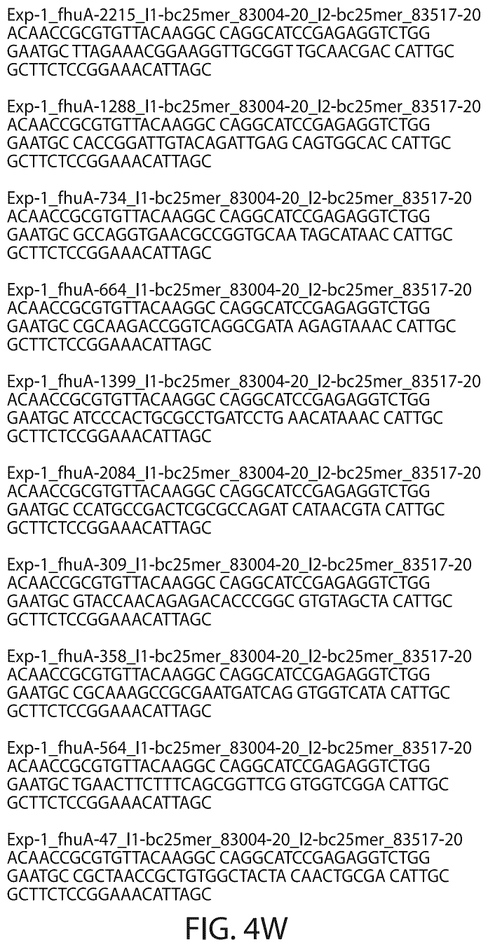

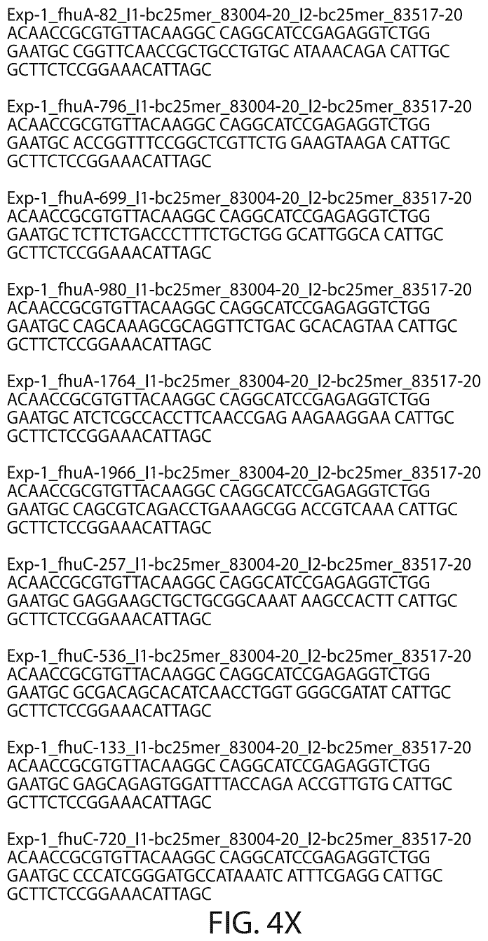

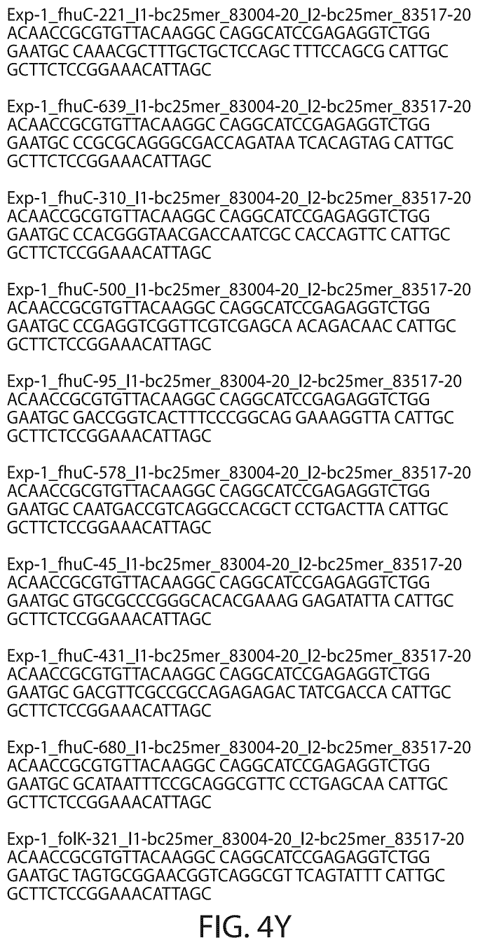

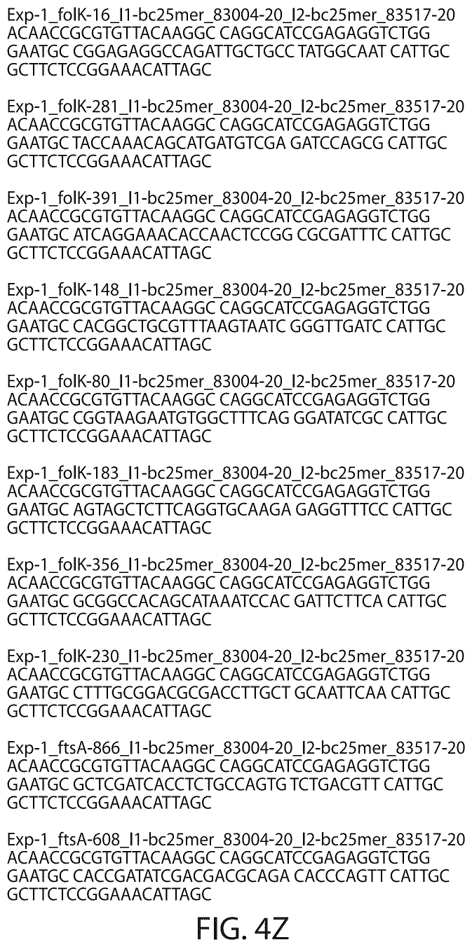

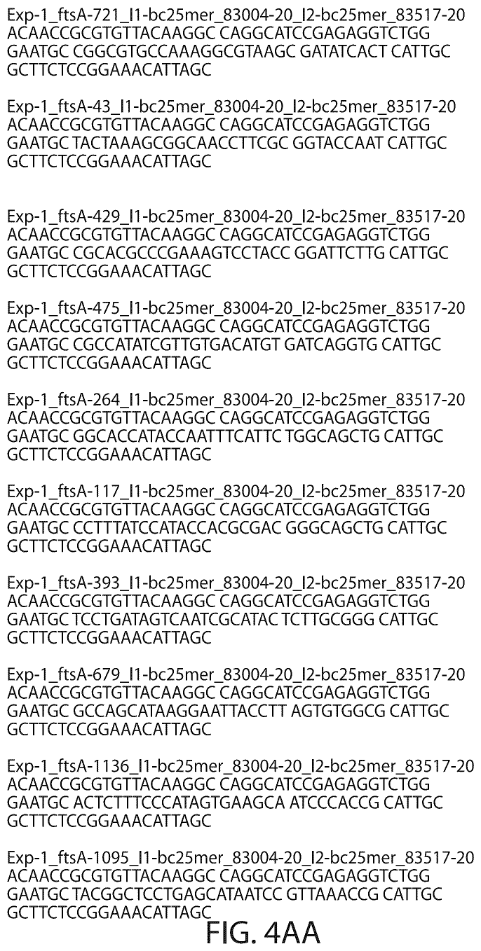

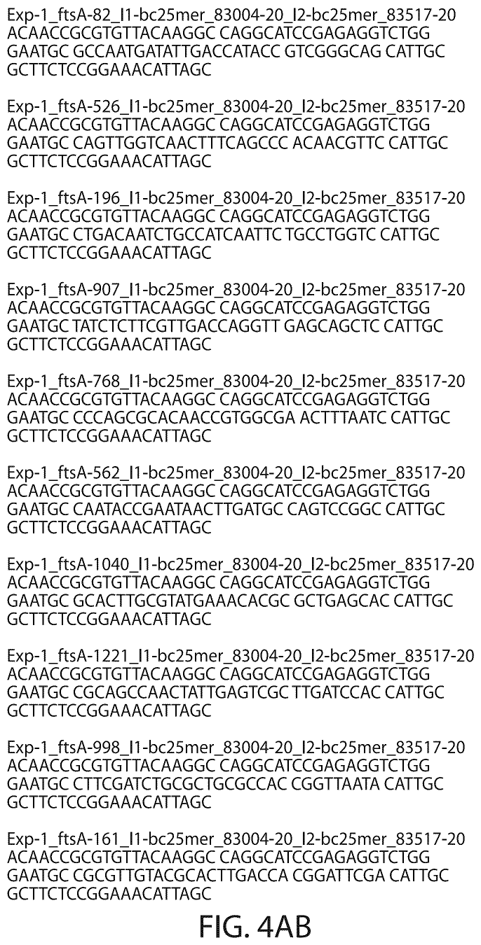

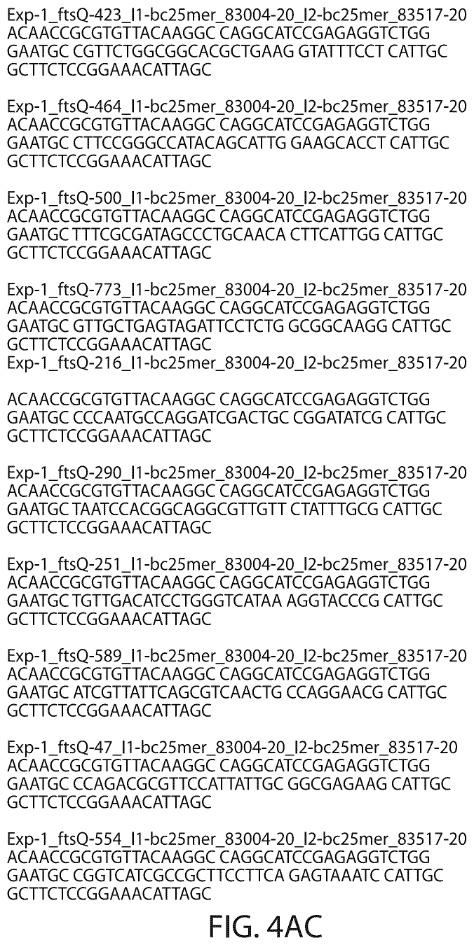

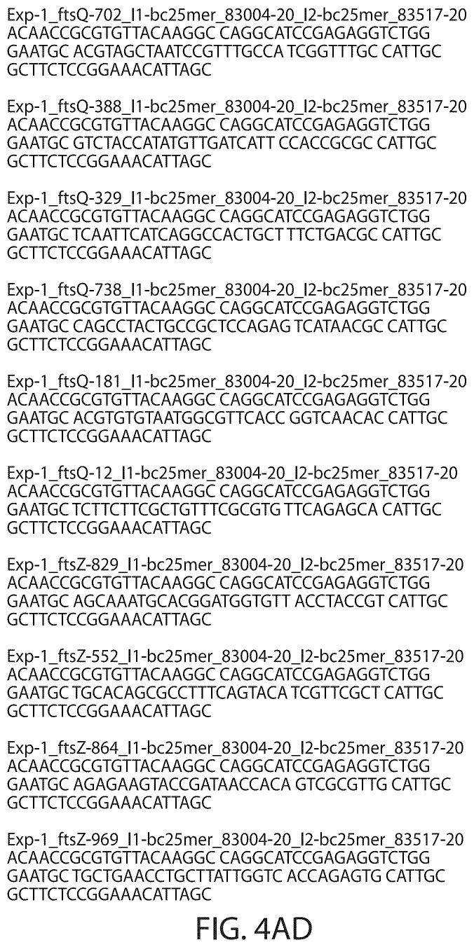

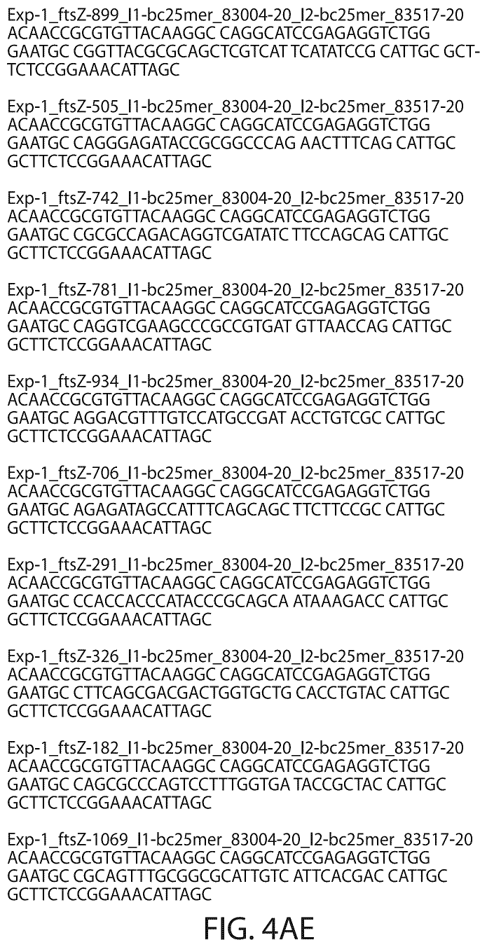

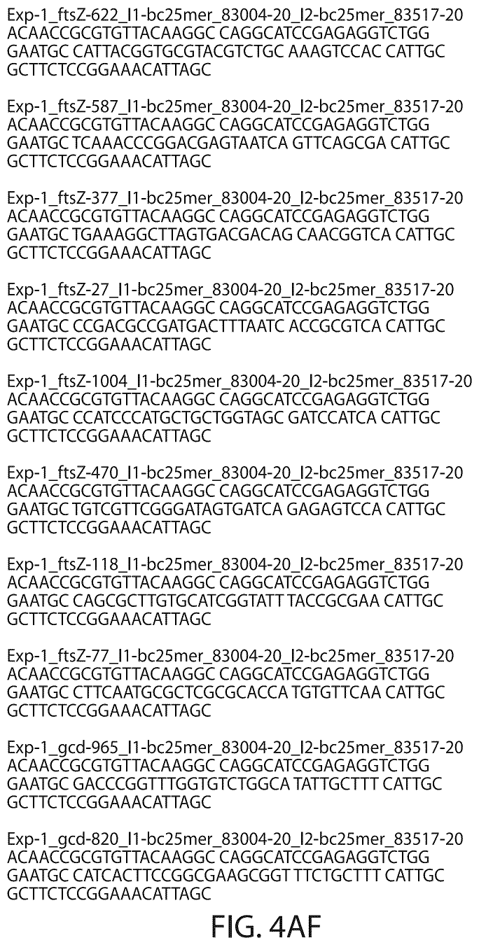

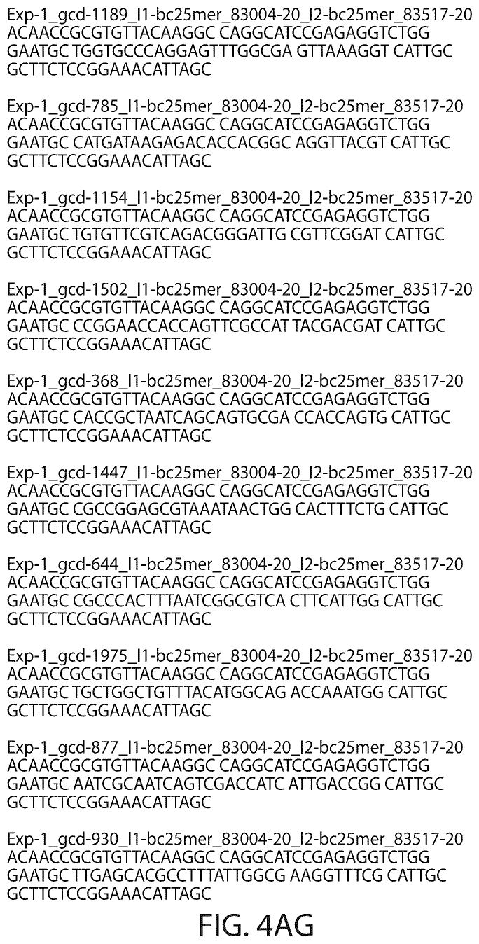

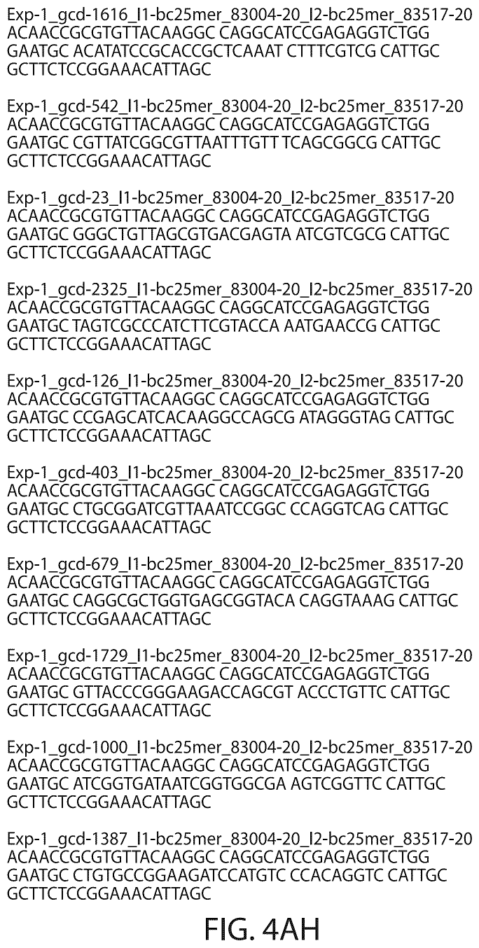

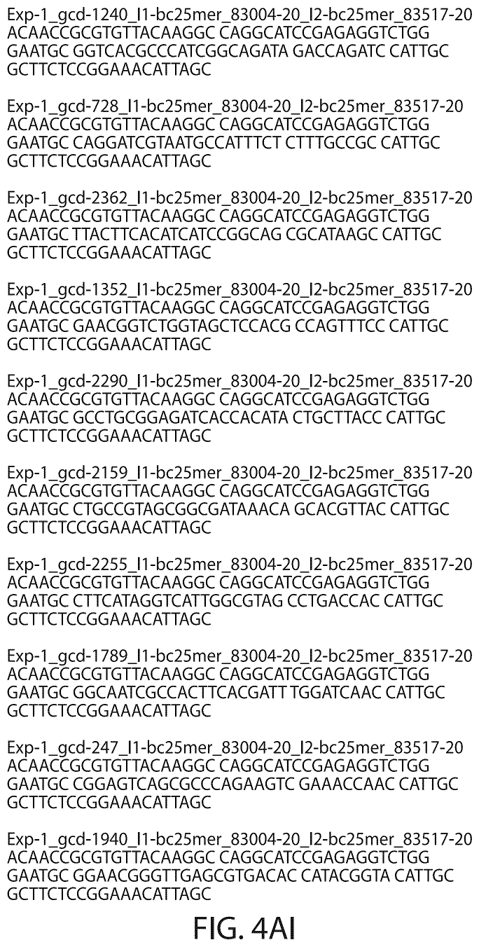

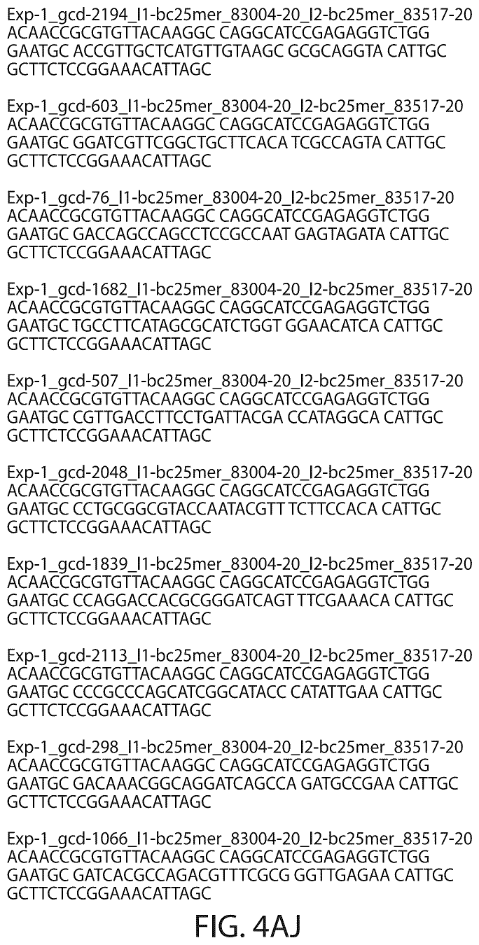

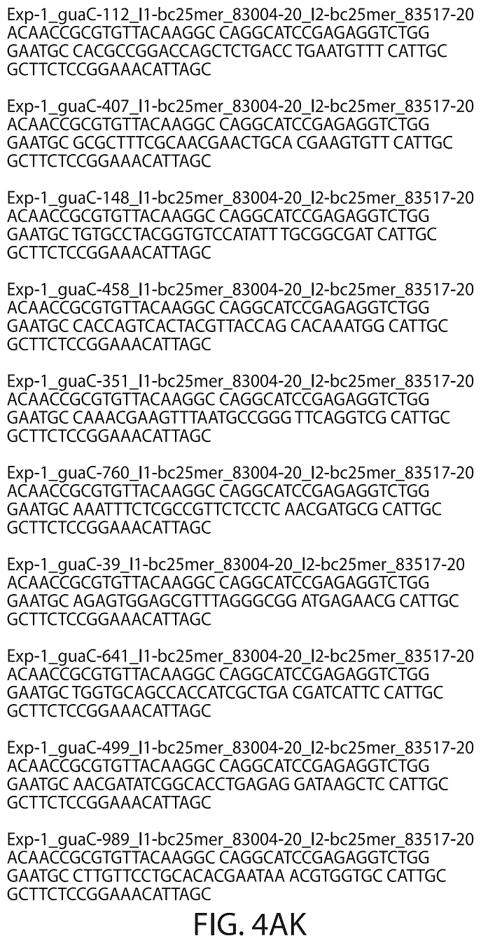

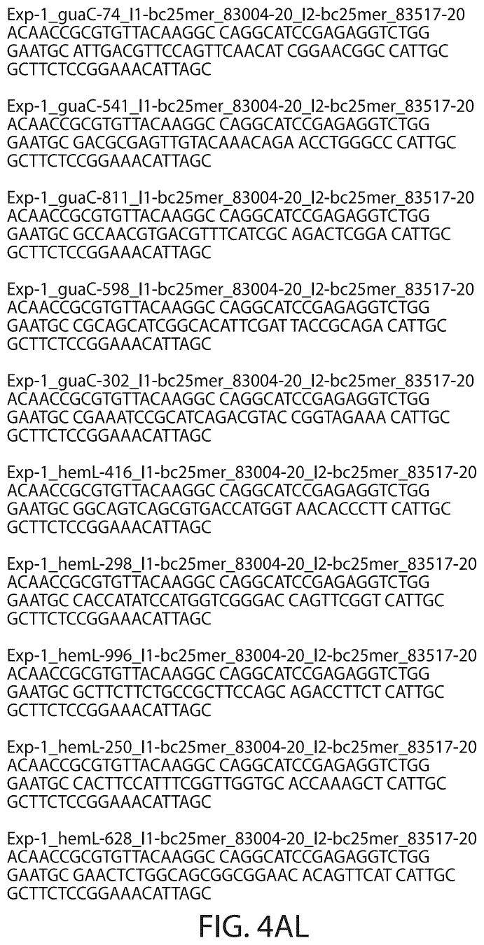

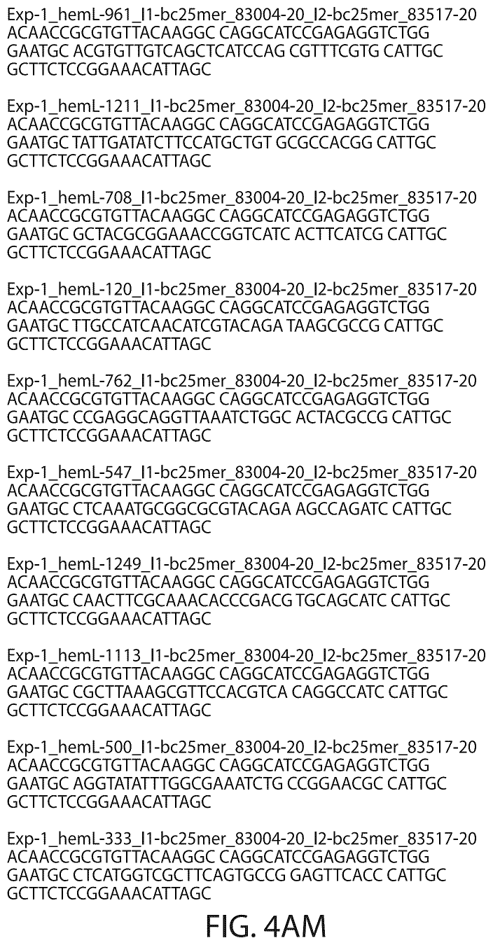

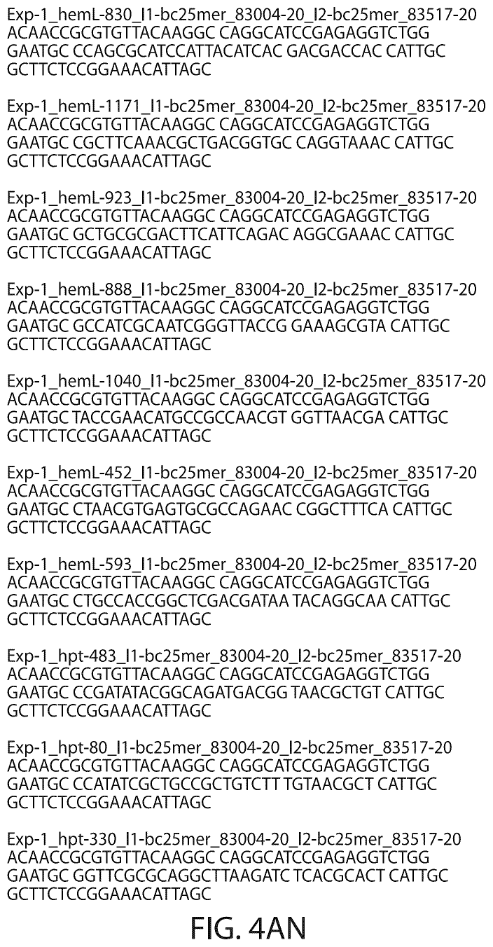

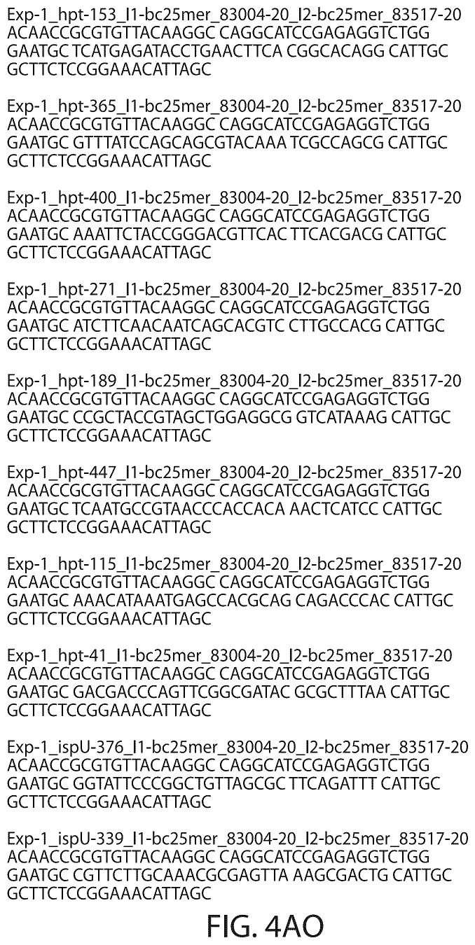

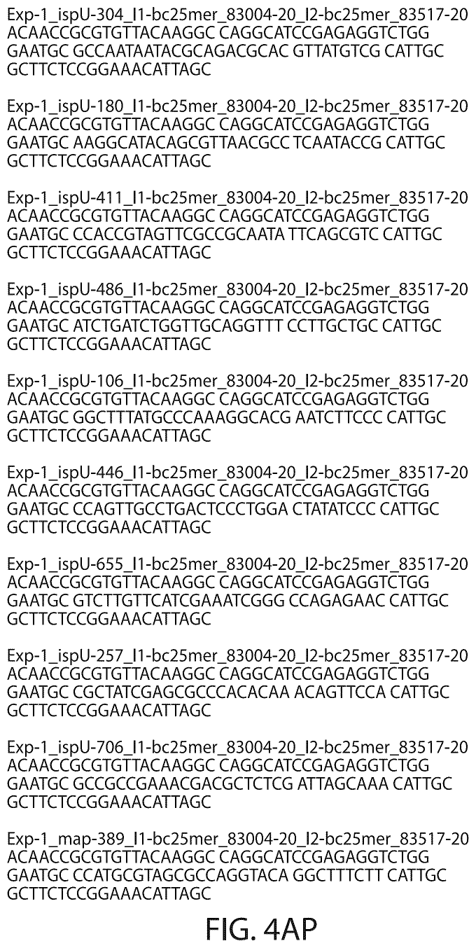

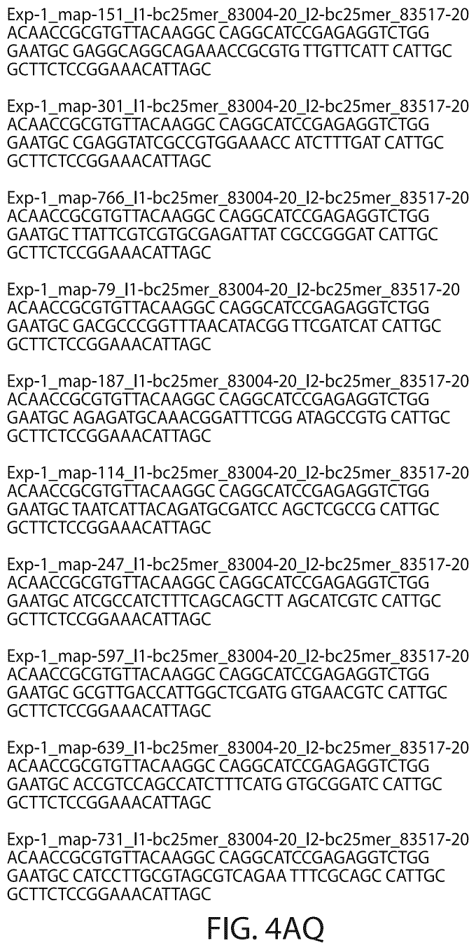

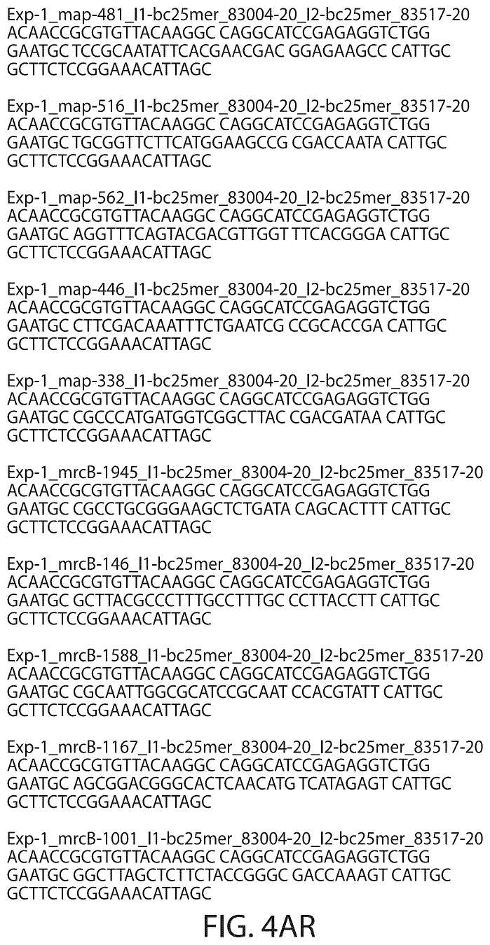

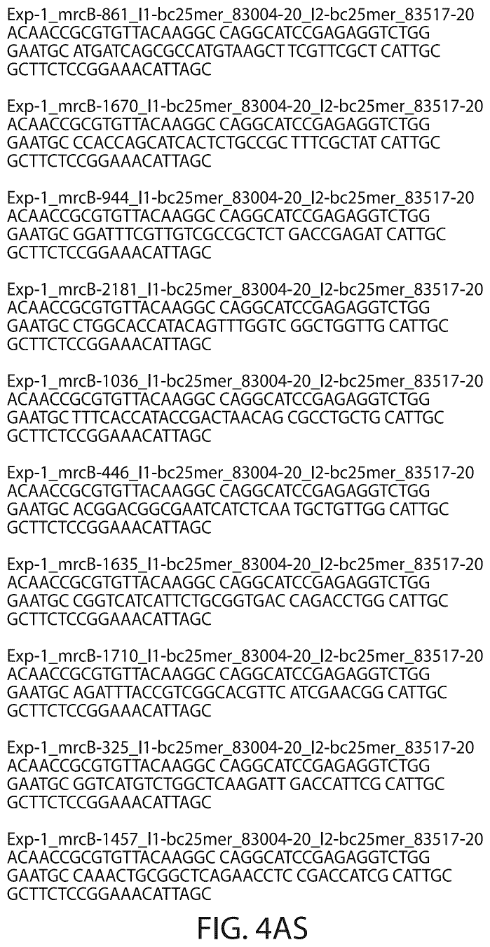

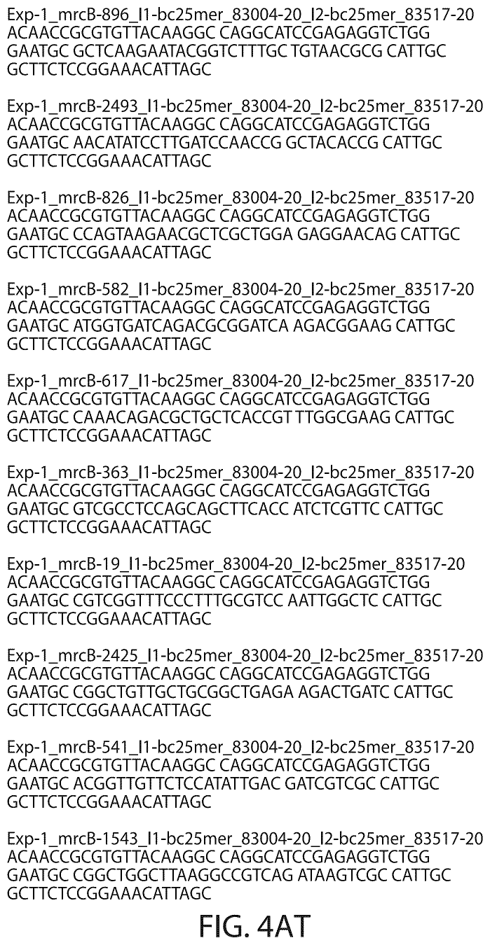

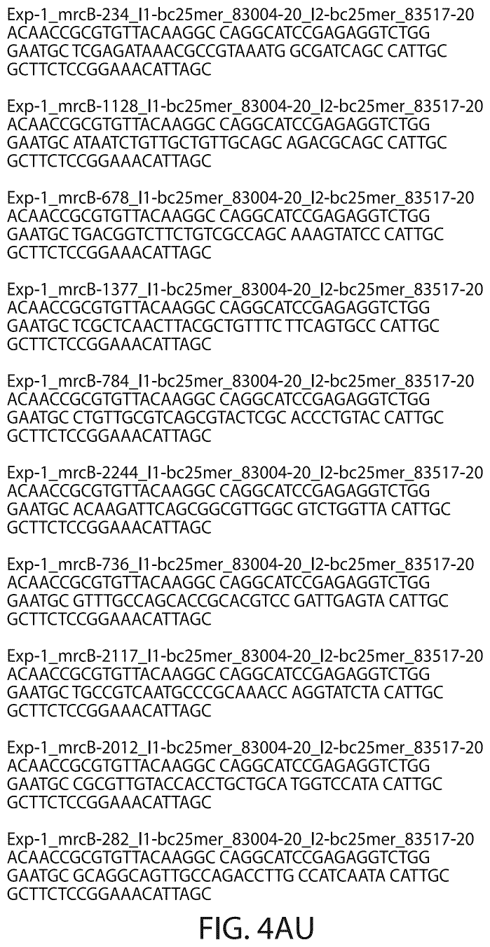

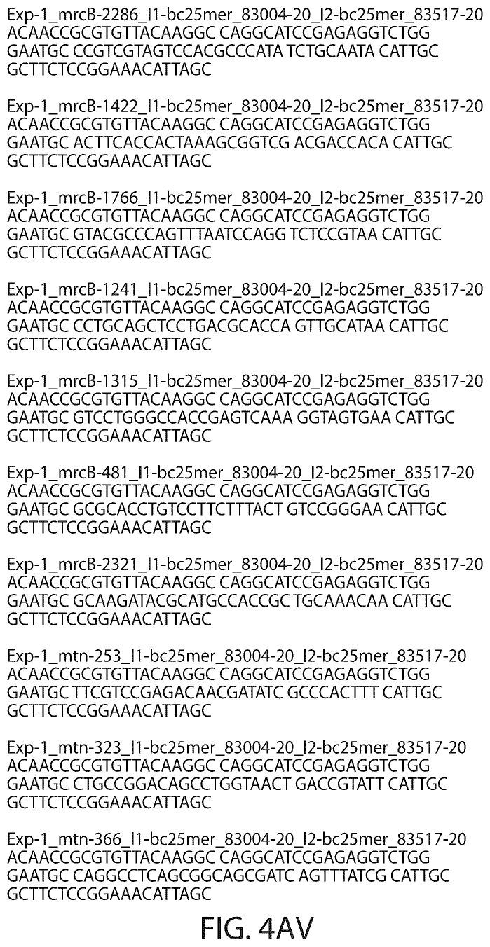

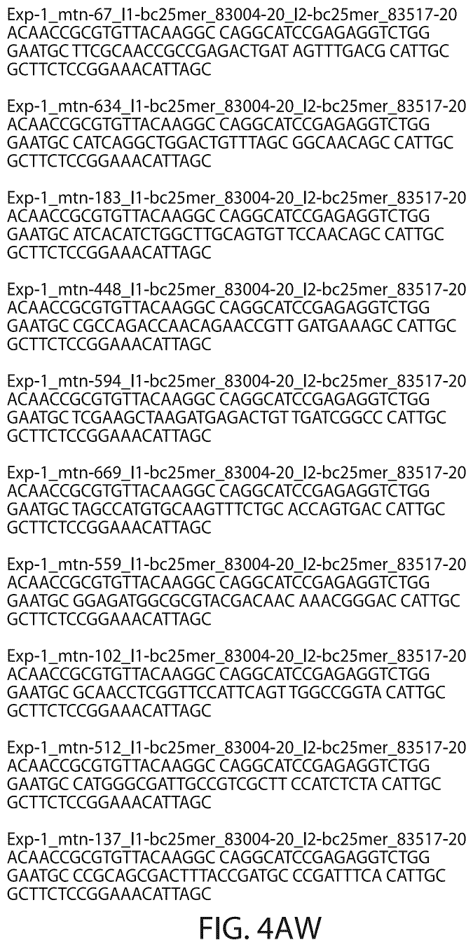

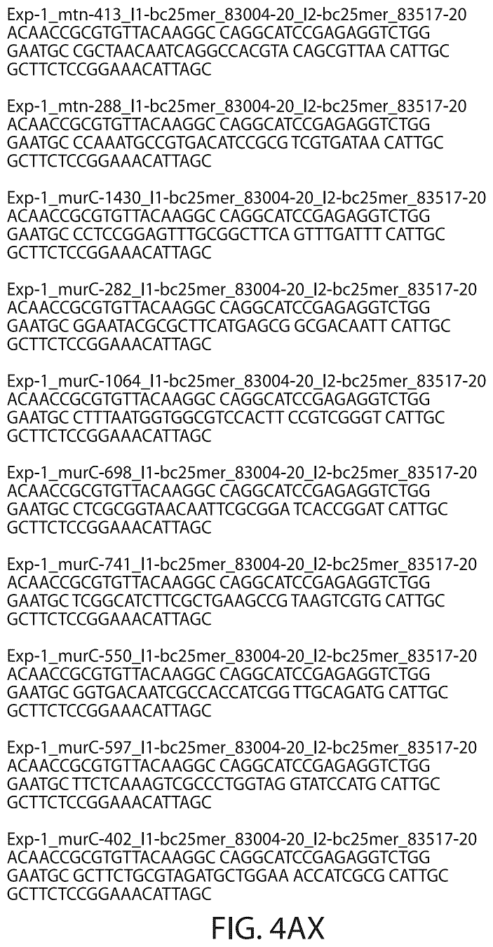

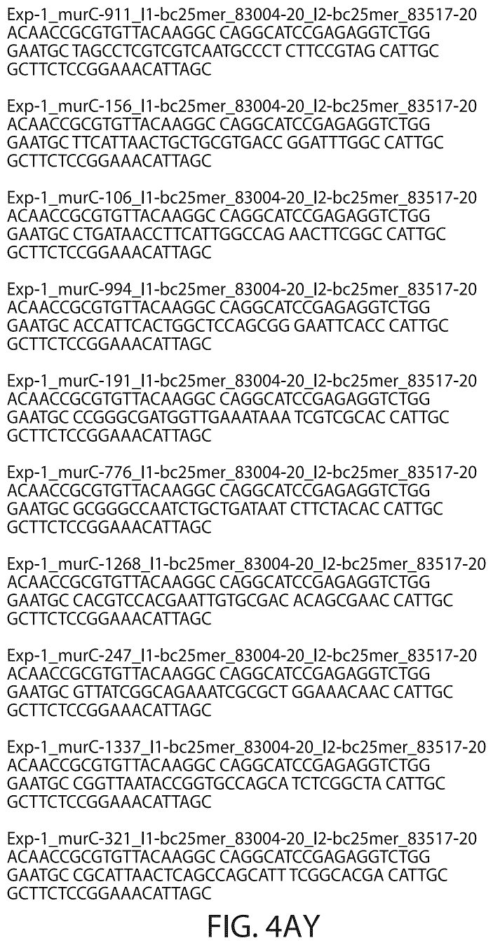

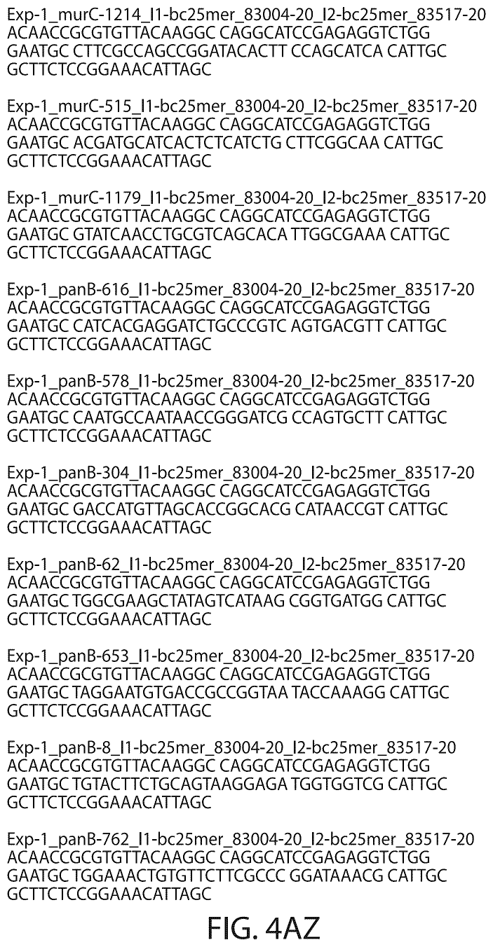

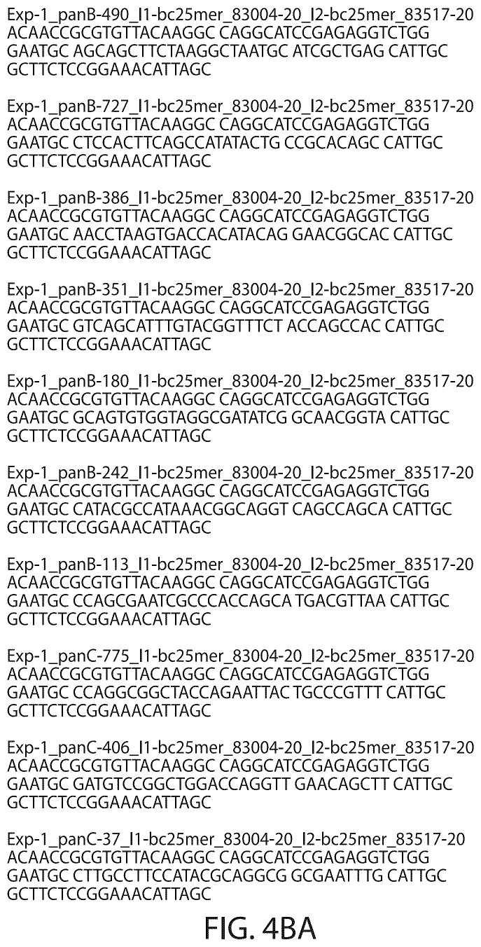

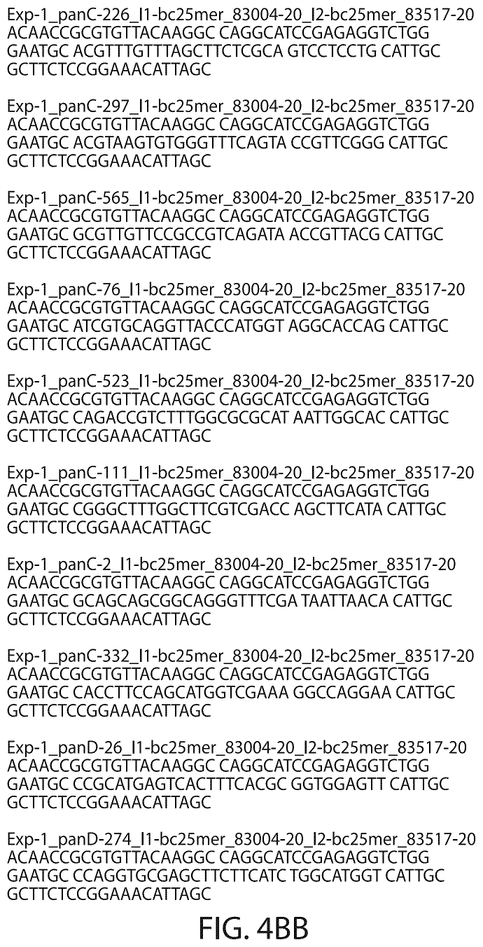

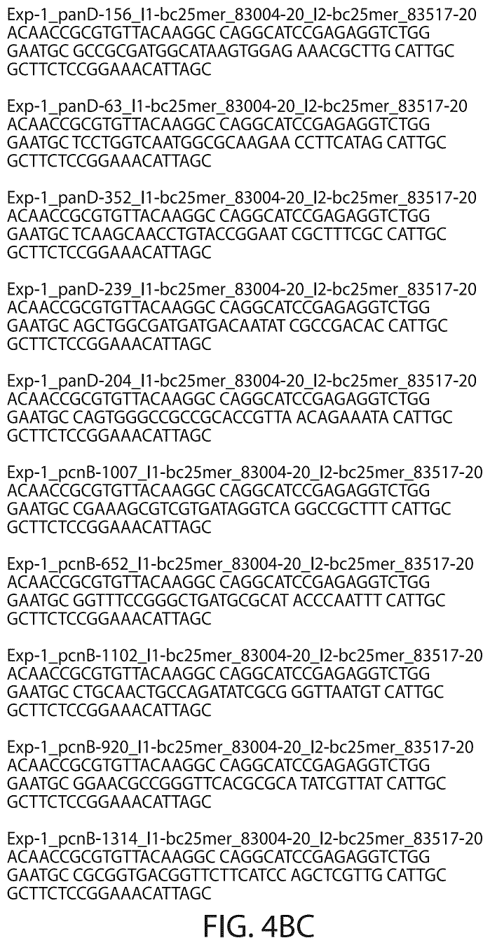

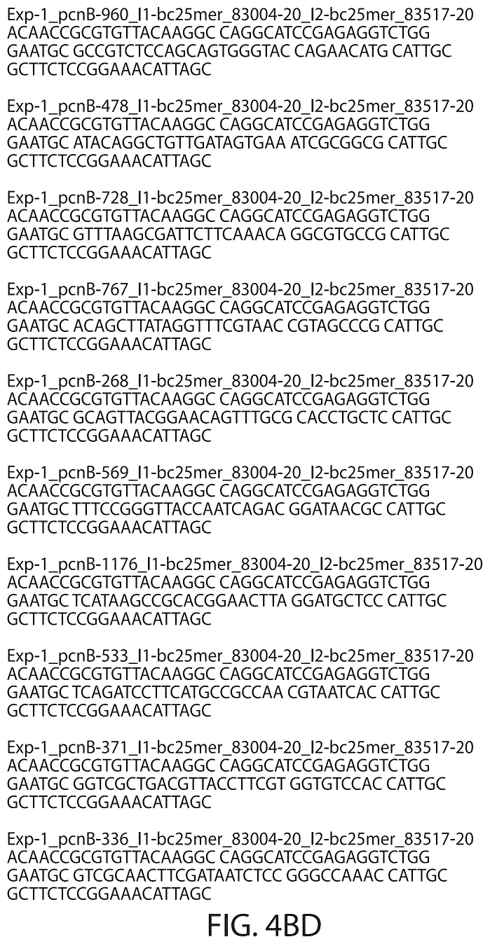

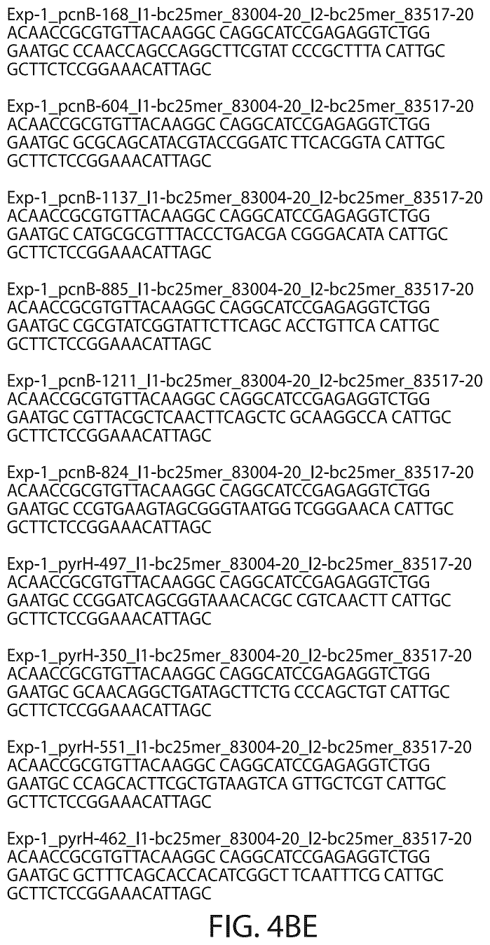

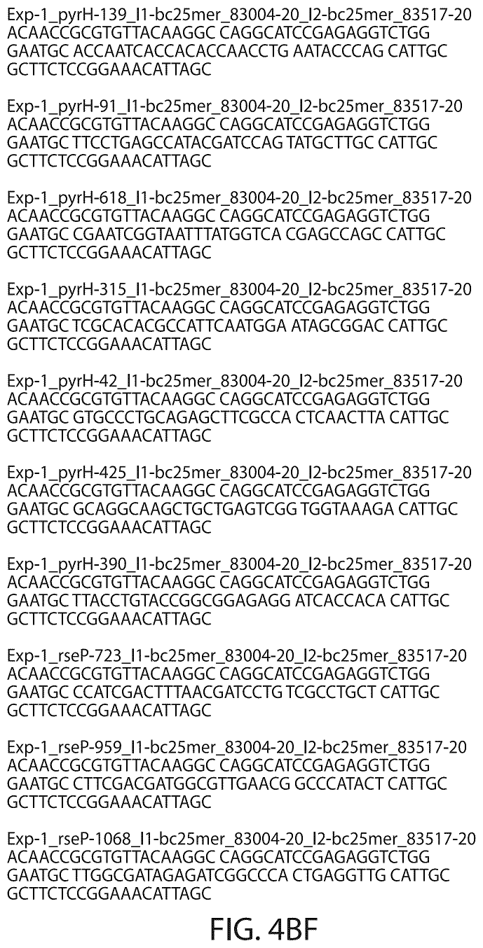

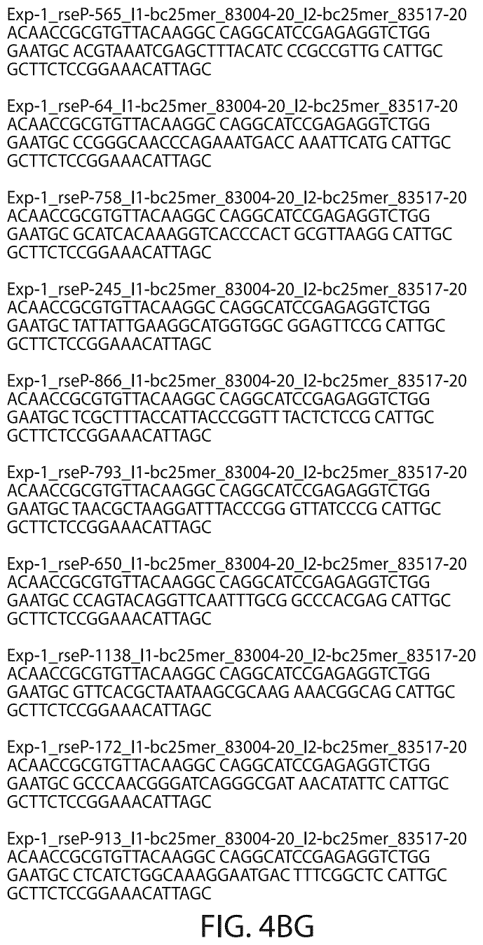

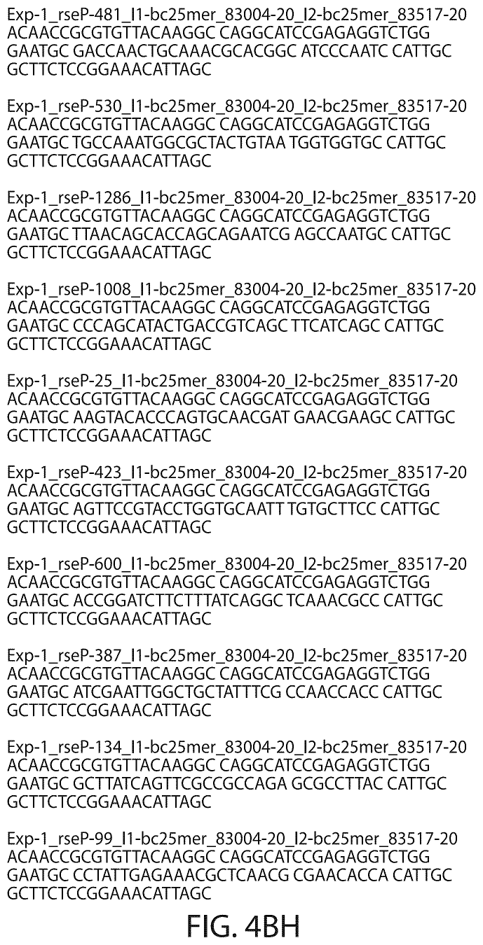

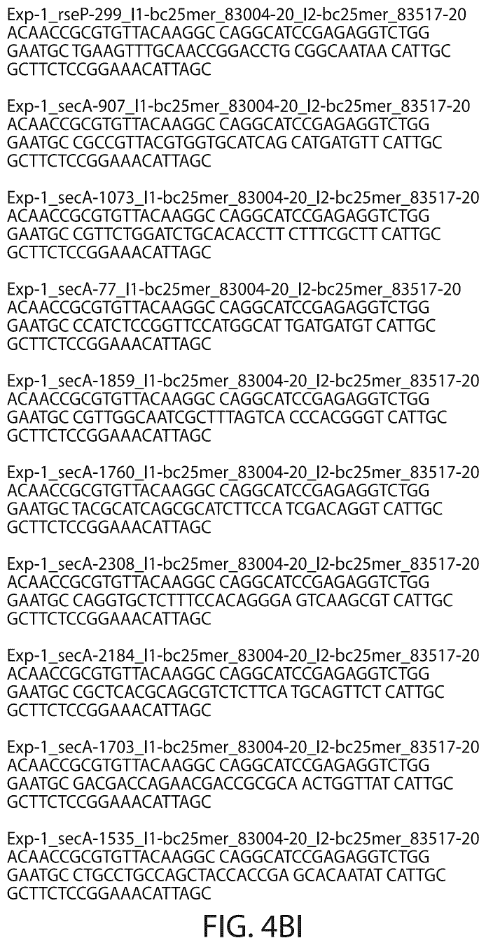

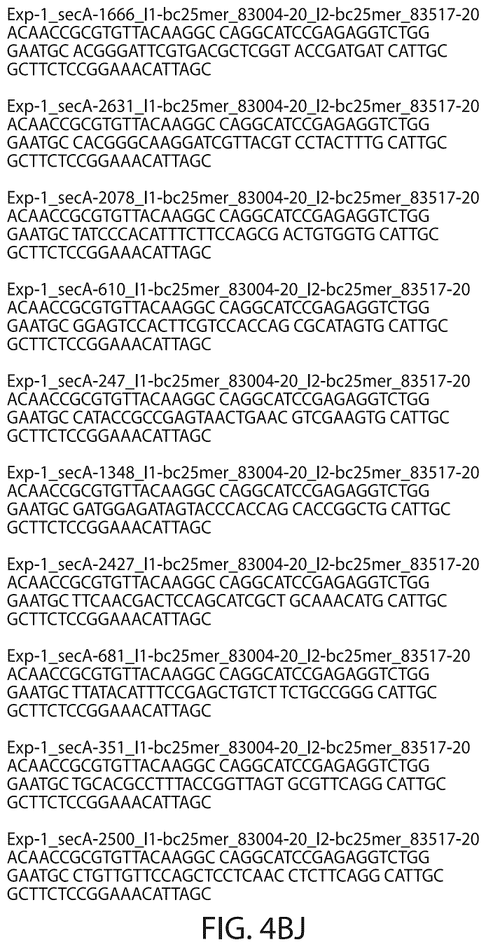

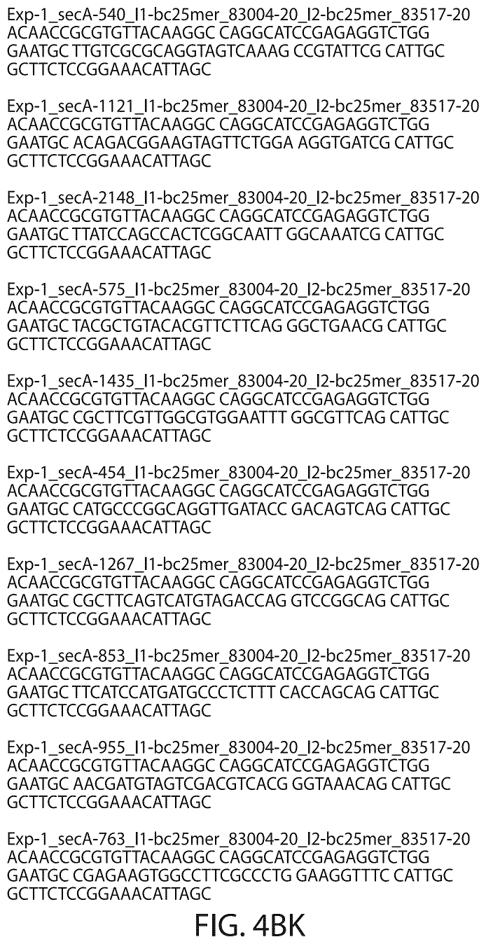

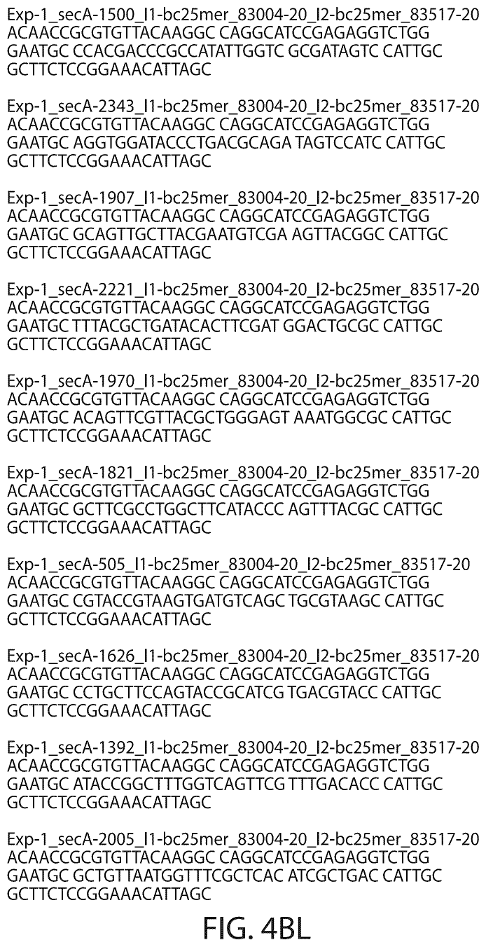

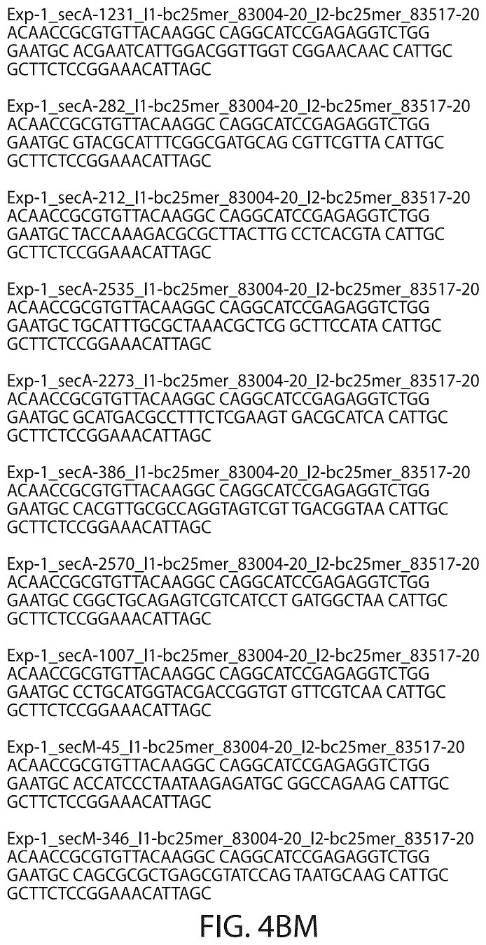

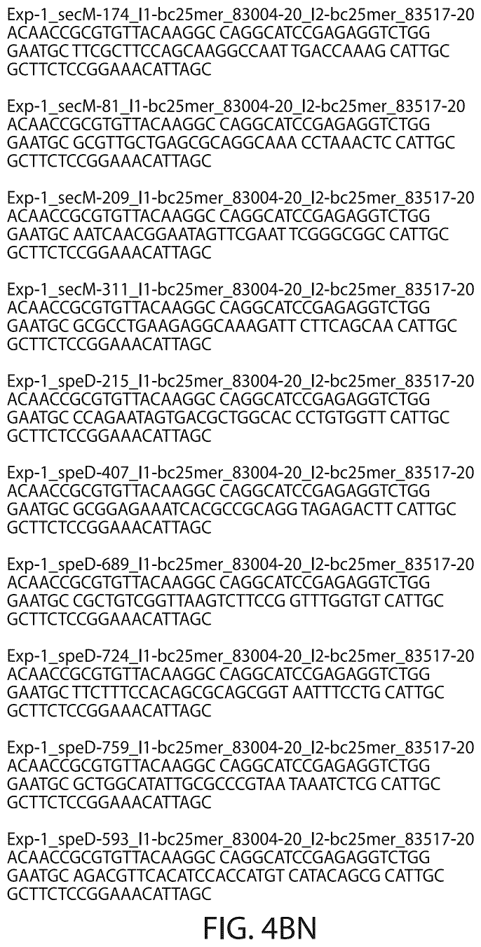

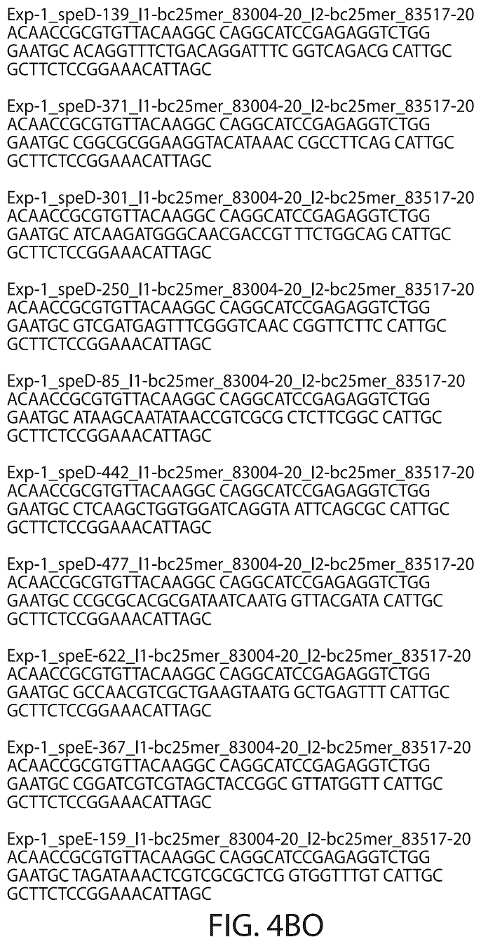

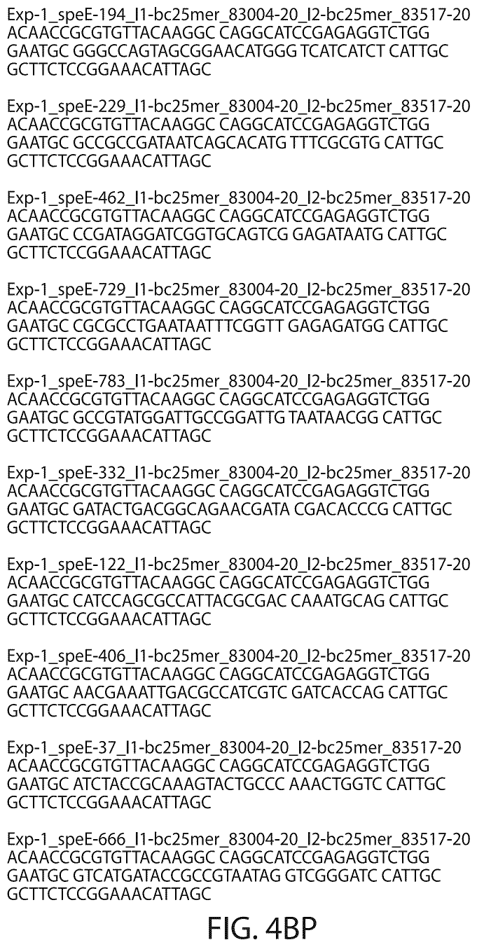

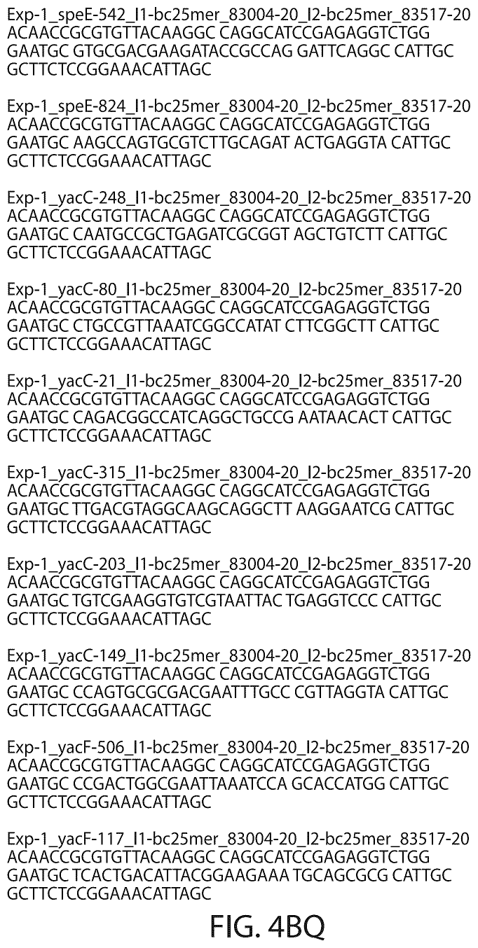

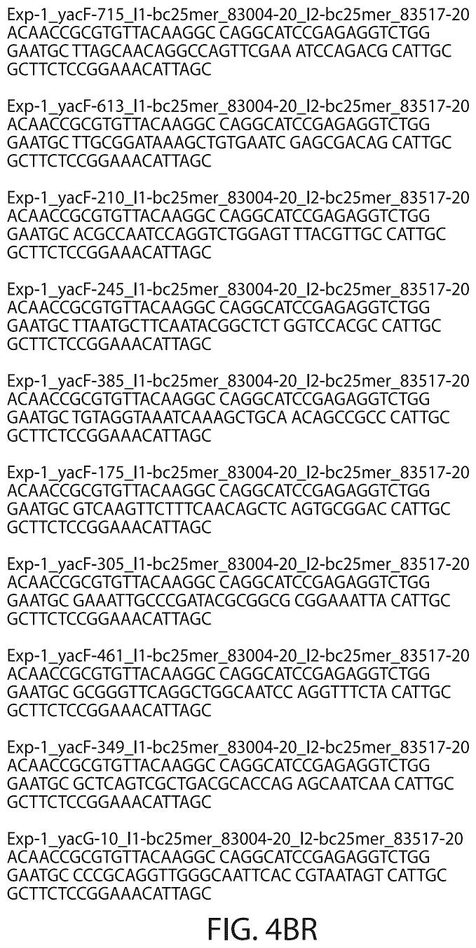

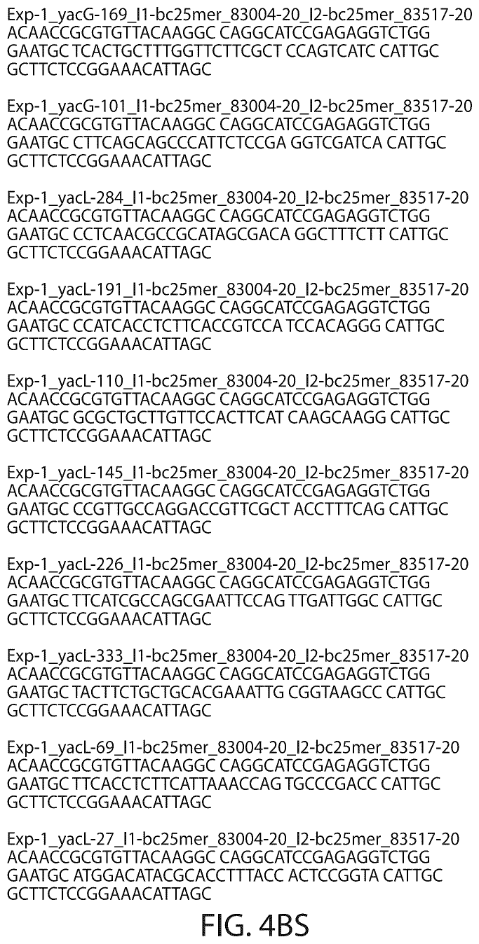

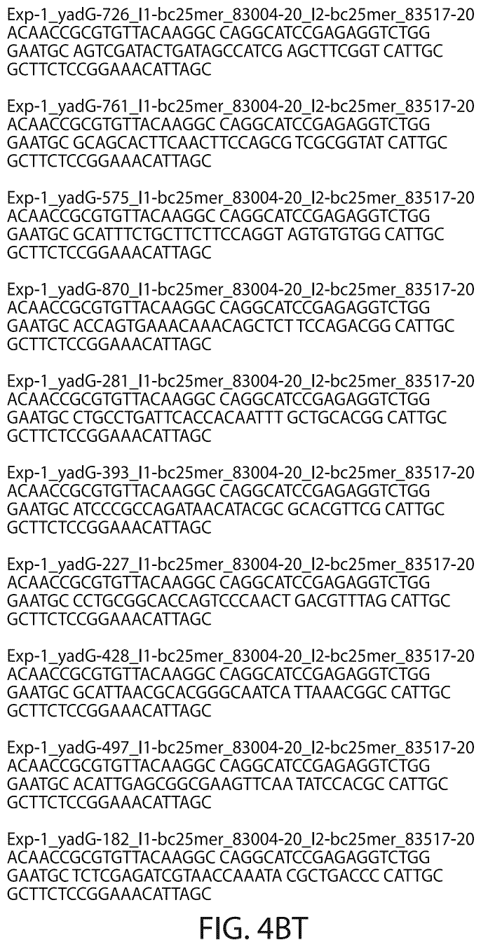

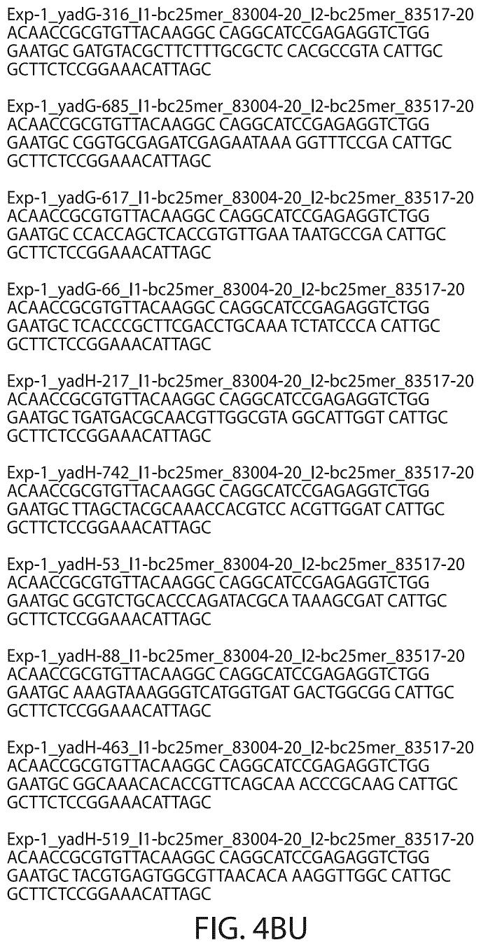

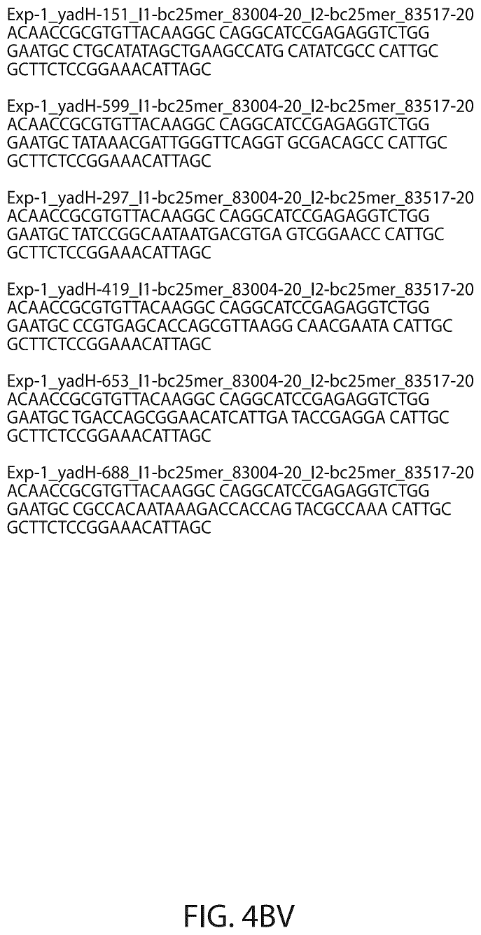

FIG. 2 shows an example template sequence containing a probe the mRNA, acnB. Underlined at the beginning is the sequence of the first primer, not underlined is the common P9 priming site, then underlined is the Nb.BsmI site, not underlined is the reverse complement to the hybridization region for acnB, next underlined is the reverse complement of the Nb.BsrDI site, and the final not underlined portion is the second unique primer. This template is one of 736 used to create probes to stain all mRNAs expressed in the copy number range of 1-10 per cell transcribed from the E. coli genomic locus corresponding to base pairs 1-100 kb. See FIGS. 4A-4BV for the sequences of the 736 probes.

Index PCR. The template for specific probe sets were selected from the complex oligopool via limited-cycle PCR. 0.5 to 1 ng of the complex oligopool was combined with 0.5 micromolar of each primer. The forward primer matched the priming sequence for the desired sub set while the reverse primer was a 5′concatenation of this sequence with the T7 promoter. To avoid the generation of G-quadruplets, which can be difficult to synthesize, the terminal Gs required in the T7 promoter were generated from Gs located at the 5′ of the priming region where appropriate. All primers were synthesized by IDT. A 50 microliter reaction volume was amplified either using the KAPA real-time library amplification kit (KAPA Biosystems; KK2701) or via a homemade qPCR mix which included 0.8× EvaGreen (Biotum; 31000-T) and the hot-start Phusion polymerase (New England Biolabs; M0535S). Amplification was followed in real time using Agilent's MX300P or Biorad's CFX Connect. Individual samples were removed before the plateau in amplification, often at concentrations about 10-fold lower than would correspond to this plateau, to minimize distortion of template abundance due to over-amplification. Individual templates were purified with columns according to the manufacturer's instructions (Zymo DNA Clean and Concentrator; D4003) and eluted in RNase-free deionized water.

Amplification via in vitro transcription. The template was then amplified via in vitro transcription. Briefly, 0.5 to 1 micrograms of template DNA was amplified into 100 to 200 micrograms of RNA in a single 20-30 microliter reaction with a high yield RNA polymerase (New England Biolabs; E2040S). Reactions were supplemented with 1× RNase inhibitor (Promega RNasin; N2611). Amplification was typically run for 2 to 4 hours at 37° C. to maximize the yield. The RNA was not purified after the reaction and was either stored at −80° C. or immediately converted into DNA as described below.

Reverse Transcription. 1-2 nmol of fluorescently-labeled ssDNA probe was created from the above in vitro transcription reactions using the reverse transcriptase Maxima H—(Thermo Scientific; EP0751). This enzyme was used because of its higher processivity and temperature resistance, which allowed the conversion of large quantities of RNA into DNA within small volumes at temperatures that disfavor secondary structure formation. The unpurified RNA created above was supplemented with 1.6 mM of each dNPT, 1-2 nmol of fluorescently labeled P9 primer, 300 units of Maxima H−, 60 units of RNasin, and a final 1× concentration of the Maxima RT buffer. The final 75 microliter volume was incubated at 50° C. for 60 minutes.

Strand Selection and Purification. The template RNA in the reaction above was then removed from the DNA via alkaline hydrolysis. 75 microliters of a solution of 0.25 M EDTA and 0.5 N NaOH was added to each reverse transcription reaction, and the sample was incubated at 95° C. for 10 minutes. The reaction was immediately neutralized by purifying the ssDNA probe with a modified version of the Zymo Oligo Clean and Concentrator protocol. Specifically, the 5-microgram capacity column was replaced with a 100-microgram capacity DNA column as appropriate. The remainder of the protocol was run according to the manufacturer's instructions. Probe was eluted in 100 microliter RNase-free deionized water and evaporated in a vacuum concentrator. The final pellet was resuspend in 10 microliter RNase-free water and stored at −20° C. Denaturing poly-acrylimid gel electrophoresis and absorption spectroscopy revealed that this protocol typically produces 90-100% incorporate of the fluorescent primer into full length probe and 60-75% recovery of the total fluorescent probe. Thus, without exceeding a 150-microliter reaction volume, this protocol can be used to create ˜2 nmol of fluorescent probe. The small reaction volumes were conducive to the use of high-throughput fluid handling techniques and significantly lower the cost of this reaction as compared to alternative approaches. Thus, 24-96 probes could be constructed in parallel, with minimal hands-on time, across two days, for a final cost of ˜$14 per 2 nmol of each probe set.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1-142. (canceled)

143. A method of producing a probe library, comprising:
simultaneously amplifying, in a common solution using limited cycle amplification, a subset of a plurality of unique oligonucleotides each comprising an identical common index region complementary to one or more PCR primers, to produce a yield of amplified unique oligonucleotides, wherein the ratio of the unique oligonucleotides, relative to each other, is substantially unchanged before and after amplification;
transcribing in vitro at least some of the amplified unique oligonucleotides to produce RNA; and,
reverse transcribing the RNA to produce transcribed DNA.

144. The method of claim 143, wherein the plurality of oligonucleotides have an average length of between 10 and 200 nucleotides.

145. The method of claim 143, wherein the plurality of oligonucleotides includes at least 100 unique oligonucleotide sequences.

146. The method of claim 143, further comprising selectively degrading the RNA after the transcribed DNA is produced.

147. The method of claim 143, wherein at least some of the amplified oligonucleotides contain a promoter.

148. The method of claim 143, wherein transcribing at least some of the amplified oligonucleotides comprises a mass of RNA that is at least 100-fold greater than the mass of amplified oligonucleotides.

149. The method of claim 143, wherein transcribing at least some of the amplified oligonucleotides comprises exposing the amplified oligonucleotides to an RNA polymerase.

150. The method of claim 143, wherein transcribing at least some of amplified oligonucleotides to produce RNA comprises producing, on average, at least 10 RNA copies of each of the amplified oligonucleotides.

151. The method of claim 143, wherein reverse transcribing the RNA comprises exposing the RNA to a reverse transcriptase.

152. The method of claim 143, wherein reverse transcribing the RNA to produce transcribed DNA occurs without first purifying the RNA from components used to produce the RNA.

153. The method of claim 143, further comprising purifying the RNA from components used to produce the RNA prior to reverse transcribing the RNA to produce transcribed DNA.

154. The method of claim 143, wherein reverse transcribing the RNA to produce transcribed DNA comprises reverse transcribing the RNA to produce transcribed DNA using a sequence containing a transcription primer.

155. The method of claim 154, wherein the sequence containing a transcription primer is incorporated into the transcribed DNA.

156. The method of claim 146, wherein selectively degrading the RNA comprises chemically reducing the RNA.

157. The method of any one of claim 143, wherein the transcribed DNA is substantially single-stranded.

158. The method of claim 143, wherein the plurality of oligonucleotides has a distribution of lengths such that no more than 10% of the oligonucleotides has a length that is less than 80% or greater than 120% of the overall average length of the plurality of nucleotides.

159. The method of claim 143, wherein the amplifying step comprises limited cycle PCR amplification.

160. The method of claim 143, wherein the amplifying step comprises controlling the amount of nucleotides or cofactors that are present in the common solution.

161. The method of claim 143, wherein the yield of amplified nucleotides is about 10-fold lower than the maximum possible amount.

162. A method of producing a probe library, comprising:
determining optimal amplification cycles for a subset of a plurality of unique oligonucleotides each comprising an identical common index region complementary to one or more PCR primers and amplified in a common solution to produce a yield of amplified unique oligonucleotides wherein the ratio of the unique oligonucleotides, relative to each other, is substantially unchanged before and after amplification;
simultaneously amplifying the plurality of unique oligonucleotides in a common solution with the optimal amplification cycles;
transcribing in vitro at least some of the amplified unique oligonucleotides to produce RNA; and,
reverse transcribing the RNA to produce transcribed DNA.
